SEQUENCE LISTING (6 sequences)

SEQ ID NO: 1 - protein, 24 aa, Homo sapiens
QLYSALANKCCHVGCTKRSLARFC

SEQ ID NO: 2 - protein, 29 aa, Homo sapiens
DSWMEEVIKLCGRELVRAQIAICGMSTWS

SEQ ID NO: 3 - protein, 185 aa, Homo sapiens
  CHAIN (25)..(53): B
MPRLFFFHLLGVCLLLNQFSRAVADSWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFC

SEQ ID NO: 4 - protein, 24 aa, Homo sapiens
DVLAGLSSSCCKWGCSKSEISSLC

SEQ ID NO: 5 - protein, 27 aa, Homo sapiens
RAAPYGVRLCGREFIRAVIFTCGGSRW

SEQ ID NO: 6 - protein, 142 aa, Homo sapiens
  CHAIN (26)..(52): B
MARYMLLLLLAVWVLTGELWPGAEARAAPYGVRLCGREFIRAVIFTCGGSRWRRSDILAHEAMGDTFPDADADEDSLAGELDEAMGSSEWLALTKSPQAFYRGRPSWQGTPGVLRGSRDVLAGLSSSCCKWGCSKSEISSLC

(19) United States
(12) Patent Application Publication
(10) Pub. No.: US 2016/0296600 A1
(43) Pub. Date: Oct. 13, 2016

(54) Relaxin Prodrugs

(71) Applicant: ASCENDIS PHARMA RELAXIN DIVISION A/S, Hellerup (DK)

(72) Inventors: Kennett Sprogøe, Palo Alto, CA (US); Felix Cleemann, Heidelberg (DE); Harald Rau, Dossenheim (DE); Nicole Hassepass, Neulussheim (DE); Thomas Wegge, Heidelberg (DE); Joachim Zettler, Dossenheim (DE); Ana Bernhard, Heidelberg (DE)

(73) Assignee: Ascendis Pharma Relaxin Division A/S, Hellerup (DK)

(21) Appl. No.: 15/035,636

(22) PCT Filed: Nov. 10, 2014
(86) PCT No.: PCT/EP2014/074114

§ 371 Date: May 10, 2016

(30) Foreign Application Priority Data
Nov. 11, 2013 (EP) 13192269.2
Apr. 9, 2014 (EP) 14164072.2

(51) Int. Cl. A61K 38/22; A61K 9/00; A61K 47/48

(52) U.S. Cl. CPC A61K 38/2221; A61K 47/48784; A61K 47/48853; A61K 9/0019; A61K 47/48215; A61K 47/48315

(57) ABSTRACT

The present invention relates to a carrier-linked relaxin prodrug, pharmaceutical compositions comprising said prodrug, their use as medicaments for the treatment of diseases which can be treated with relaxin, methods of application of such carrier-linked relaxin prodrug or pharmaceutical compositions, methods of treatment, and containers comprising such prodrug or compositions.

The present invention relates to a carrier-linked relaxin prodrug, pharmaceutical compositions comprising said prodrug, their use as medicaments for the treatment of diseases which can be treated with relaxin, methods of application of such carrier-linked relaxin prodrug or pharmaceutical compositions, methods of treatment, and containers comprising such prodrug or compositions.

Mature human relaxin is a hormonal peptide of approximately 6000 daltons known to be responsible for remodeling the reproductive tract before parturition, thus facilitating the birth process. This protein appears to modulate the restructuring of connective tissues in target organs to obtain the required changes in organ structure during pregnancy and parturition.

Circulating levels of relaxin are elevated for the entire nine months of pregnancy and drop quickly following delivery. While predominantly a hormone of pregnancy, relaxin has also been detected in the non-pregnant female as well as in the male. Recently, relaxin has been found to be useful in the treatment of heart failure and may be beneficial for treating a number of human diseases, including but not limited to acute and chronic heart failure, compensated heart failure, staple heart failure, dyspnea, dyspnea associated with heart failure, preeclampsia, eclampsia, hypertension, fibrosis, bone disease, cancer, cervical ripening, induction of labor, sclerosis, scleroderma, pulmonary, renal, and hepatic fibrosis, tooth movement, hepatic impairment, compensated cirrhosis and portal hypertension, pulmonary hypertension, pulmonary arterial hypertension, end stage renal disease, pancreatitis, and inflammation-related diseases like rheumatoid arthritis (see for example: Teerlink et al. The Lancet, 2013, Volume 381, Issue 9860, Pages 29-39; Cemaro et al. Med Res Rev. 2013 published ahead of print 11 Feb. 2013; Tozzi et al, Pulmonary Pharmacology and Therapeutics, 2005, 18, 346-53; Bennett R G. 2009, Transl Res. 154(1): 1-6.; Santora et al, Journal of pharmacology and experimental therapeutics, 2007, Vol. 322(2), 887-893; Cosen-Binker et al. 2006 World J Gastroenterol 2006 Mar. 14; 12(10): 1558-1568).

As relaxin increases arterial compliance, relaxin may also be administered to subjects suffering from one or more of the following disorders: atherosclerosis, Type 1 diabetes, Type 2 diabetes, coronary artery disease, scleroderma, stroke, diastolic dysfunction, familial hypercholesterolemia, isolated systolic hypertension, primary hypertension, secondary hypertension, left ventricular hypertrophy, arterial stiffness associated with long-term tobacco smoking, arterial stiffness associated with obesity, arterial stiffness associated with age, systemic lupus erythematosus, preeclampsia, and hypercholesterolemia. Furthermore, relaxin may also be administered to increase arterial compliance in perimenopausal, menopausal, and post-menopausal women and in individuals who are at risk of one of the aforementioned.

The half-life of intravenously administrated Relaxin in humans is less than 10 minutes (Dschietzig T. et al. Journal of Cardiac Failure, 2009, 15(3), 182-190). As a consequence, Relaxin has to be administered as continuous intravenous infusions, typically for at least 48 hours. This limits relaxin applicability in diseases where continuous infusion is neither feasible nor practicable. There is a large need for therapeutics based on relaxin with longer duration of action, and improved route of administration, and the current invention meets, among other things, this objective.

In view of the above, there exists a need to provide a form of administration that overcomes these drawbacks at least partially.

Therefore, it is an object of the present invention to develop long-acting relaxin prodrugs which at least partially overcome the before mentioned shortcomings.

This object is achieved with a carrier-linked relaxin prodrug or pharmaceutically acceptable salt thereof comprising at least one relaxin moiety covalently connected to a carrier moiety via a reversible linker moiety.

Such carrier-linked relaxin prodrug or pharmaceutically acceptable salt thereof of the present invention provide sustained relaxin release from a subcutaneous or locally applied depot and can thus overcome at least some of the above-mentioned shortcomings.

Within the present invention the terms are used having the meaning as follows.

As used herein, the term “hydrogel” means a hydrophilic or amphiphilic polymeric network composed of homopolymers or copolymers, which is insoluble due to the presence of covalent chemical crosslinks. The crosslinks provide the network structure and physical integrity.

As used herein, the term “reagent” means a chemical compound which comprises at least one functional group for reaction with the functional group of another reagent or moiety.

As used herein, the term “backbone reagent” means a reagent, which is suitable as a starting material for forming hydrogels. As used herein, a backbone reagent preferably does not comprise biodegradable linkages. A backbone reagent may comprise a “branching core” which refers to an atom or moiety to which more than one other moiety is attached.

As used herein, the term “crosslinker reagent” means a linear or branched reagent, which is suitable as a starting material for crosslinking backbone reagents. Preferably, the crosslinker reagent is a linear chemical compound. Preferably, a crosslinker reagent comprises at least one biodegradable linkage.

As used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “—” indicates attachment to another moiety. Accordingly, a biologically active moiety is released from a prodrug as a drug, i.e. relaxin moiety is released from the carrier-linked relaxin prodrug of the present invention as relaxin.

Accordingly, the phrase “in bound form” is used to refer to the corresponding moiety of a reagent, i.e. “lysine in bound form” refers to a lysine moiety which lacks one or more atom(s) of the lysine reagent and is part of a molecule.

As used herein, the term “functional group” means a group of atoms which can react with other functional groups. Functional groups include but are not limited to the following groups: carboxylic acid (—(C═O)OH), primary or secondary amine (—NH₂, —NH—), maleimide, thiol (—SH), sulfonic acid (—(O═S═O)OH), carbonate, carbamate (—O(C═O)N<), hydroxy (—OH), aldehyde (—(C═O)H), ketone (—(C═O)—), hydrazine (>N—N<), isocyanate, isothiocyanate, phosphoric acid (—O(P═O)OHOH), phosphonic acid (—O(P═O)OHH), haloacetyl, alkyl halide, acryloyl, aryl fluoride, hydroxylamine, disulfide, vinyl sulfone, vinyl ketone, diazoalkane, oxirane, and aziridine.

As used herein, the term “activated functional group” means a functional group, which is connected to an activating group, i.e. a functional group was reacted with an activating reagent. Preferred activated functional groups include but are not limited to activated ester groups, activated carbamate groups, activated carbonate groups and activated thiocarbonate groups. Preferred activating groups are selected from the group consisting of formulas ((f-i) to (f-vi):

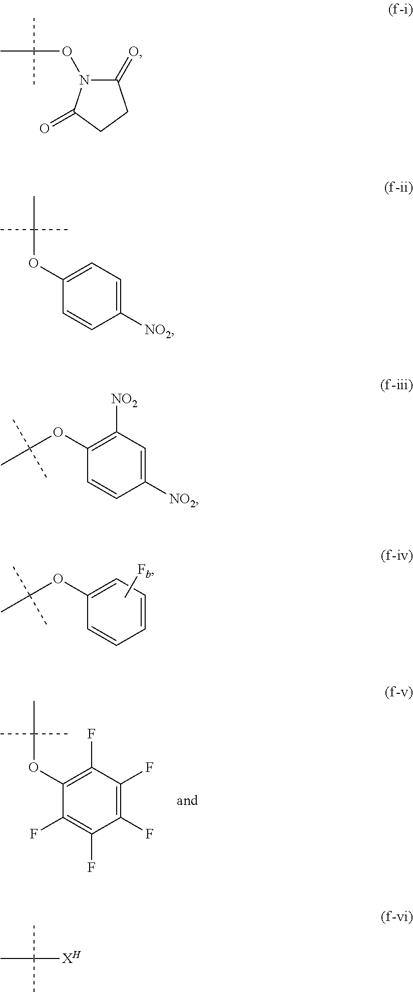

wherein
the dashed lines indicate attachment to the rest of the molecule;
b is 1, 2, 3 or 4; and
X^{H} is Cl, Br, I, or F.

Accordingly, a preferred activated ester has the formula
—(C═O)—Y¹,
wherein
Y¹ is selected from the group consisting of formulas (f-i), (f-ii), (f-iii), (f-iv), (f-v) and (f-vi).

Accordingly, a preferred activated carbamate has the formula
—N—(C═O)—Y¹,
wherein
Y¹ is selected from the group consisting of formulas (f-i), (f-ii), (f-iii), (f-iv), (f-v) and (f-vi).

Accordingly, a preferred activated carbonate has the formula
—O—(C═O)—Y¹,
wherein
Y¹ is selected from the group consisting of formulas (f-i), (f-ii), (f-iii), (f-iv), (f-v) and (f-vi).

Accordingly, a preferred activated thiocarbonate has the formula
—S—(C═O)—Y¹
wherein
Y¹ is selected from the group consisting of formulas (f-i), (f-ii), (f-iii), (f-iv), (f-v) and (f-vi).

As used herein, the term “peptide” refers to a chain of two to fifty amino acid monomers linked by peptide bonds. As used herein, the term “protein” refers to a chain of more than fifty amino acid monomers linked by peptide bonds. Preferably, a protein comprises less than 10000 amino acids monomers, such as no more than 5000 amino acid monomers or no more than 2000 amino acid monomers.

As used herein, the term “polymer” means a molecule comprising repeating structural units, i.e. the monomers, connected by chemical bonds in a linear, circular, branched, crosslinked or dendrimeric way or a combination thereof, which may be of synthetic or biological origin or a combination of both. It is understood that a polymer may for example also comprise functional groups or capping moieties. Preferably, a polymer has a molecular weight of at least 0.5 kDa, e.g. a molecular weight of at least 1 kDa, a molecular weight of at least 2 kDa, a molecular weight of at least 3 kDa or a molecular weight of at least 5 kDa.

As used herein, the term “polymeric” means a reagent or a moiety comprising one or more polymer(s).

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers. As used herein, the term “number average molecular weight” means the ordinary arithmetic means of the molecular weights of the individual polymers.

As used herein, the term “polymerization” or “polymerizing” means the process of reacting monomer or macromonomer reagents in a chemical reaction to form polymer chains or networks, including but not limited to hydrogels.

As used herein, the term “macromonomer” means a molecule that was obtained from the polymerization of monomer reagents.

As used herein, the term “condensation polymerization” or “condensation reaction” means a chemical reaction, in which the functional groups of two reagents react to form one single molecule, i.e. the reaction product, and a low molecular weight molecule, for example water, is released.

As used herein, the term “suspension polymerization” means a heterogeneous and/or biphasic polymerization reaction, wherein the monomer reagents are dissolved in a first solvent, forming the disperse phase which is emulsified in a second solvent, forming the continuous phase. In the present invention, the monomer reagents are the at least one backbone reagent and the at least one crosslinker reagent. Both the first solvent and the monomer reagents are not soluble in the second solvent. Such emulsion is formed by stirring, shaking, exposure to ultrasound or Microsieve™ emulsification, more preferably by stirring or Microsieve™ emulsification and more preferably by stirring. This emulsion is stabilized by an appropriate emulsifier. The polymerization may be initiated by addition of a base as initiator which is soluble in at least the first solvent. A suitable commonly known base suitable as initiator may be a tertiary base, such as tetramethylethylenediamine (TMEDA).

As used herein, the term “immiscible” means the property where two substances are not capable of combining to form a homogeneous mixture.

As used herein, the term “polyamine” means a reagent or moiety comprising more than one amine (—NH— and/or —NH₂), e.g. from 2 to 64 amines, from 4 to 48 amines, from 6 to 32 amines, from 8 to 24 amines, or from 10 to 16 amines. Particularly preferred polyamines comprise from 2 to 32 amines.

As used herein, the term “PEG-based comprising at least X % PEG” in relation to a moiety or reagent means that said moiety or reagent comprises at least X % (w/w) ethylene glycol units (—CH₂CH₂O—), wherein the ethylene glycol units may be arranged blockwise, alternating or may be randomly distributed within the moiety or reagent and preferably all ethylene glycol units of said moiety or reagent are present in one block; the remaining weight percentage of the PEG-based moiety or reagent are other moieties especially selected from the following moieties and linkages:
C₁₋₅₀ alkyl, C₂₋₅₀ alkenyl, C₂₋₅₀ alkynyl, C₃₋₁₀ cycloalkyl, 4- to 7-membered heterocyclyl, 8- to 11-membered heterobicyclyl, phenyl; naphthyl; indenyl; indanyl; and tetralinyl; and
linkages selected from the group comprising

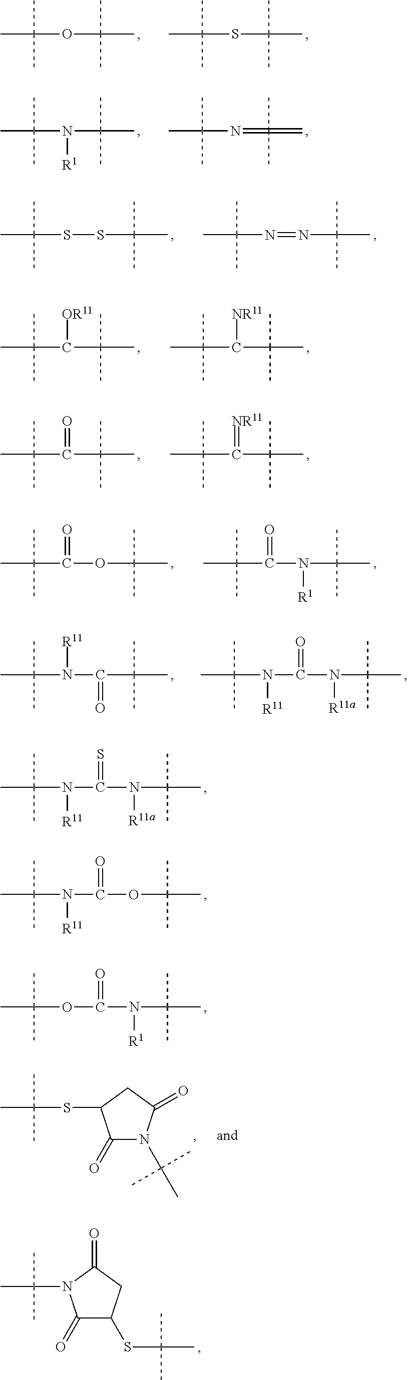

wherein
dashed lines indicate attachment to the remainder of the moiety or reagent, and
R¹¹ and R^{11a} are independently of each other selected from H and C₁₋₆ alkyl.

As used herein, the term “C₁₋₄ alkyl” alone or in combination means a straight-chain or branched alkyl group having 1 to 4 carbon atoms. If present at the end of a molecule, examples of straight-chain and branched C₁₋₄ alkyl groups are methyl, ethyl, n-propyl, isopropyl, n-butyl, isobutyl, sec-butyl and tert-butyl. When two moieties of a molecule are linked by the C₁₋₄ alkyl group, then examples for such C₁₋₄alkyl groups are —CH₂—, —CH₂—CH₂—, —CH(CH₃)—, —CH₂—CH₂—CH₂—, —CH(C₂H₅)—, —C(CH₃)₂—, —CH₂—CH₂—CH₂—CH₂—, and —CH₂—CH₂—CH₂(CH₃)—. Each hydrogen atom of a C₁₋₄ alkyl group may be replaced by a substituent as defined below. Optionally, a C₁₋₄ alkyl may be interrupted by one or more moieties as defined below.

As used herein, the term “C₁₋₆ alkyl” alone or in combination means a straight-chain or branched alkyl group having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆ alkyl group may be replaced by a substituent as defined below. Optionally, a C₁₋₆ alkyl may be interrupted by one or more moieties as defined below.

Accordingly, as used herein, the term “C₁₋₂₀ alkyl” alone or in combination means a straight-chain or branched alkyl group having 1 to 20 carbon atoms. The term “C₈₋₁₈ alkyl” alone or in combination means a straight-chain or branched alkyl group having 8 to 18 carbon atoms. Accordingly, as used herein, the term “C₁₋₅₀ alkyl” alone or in combination means a straight-chain or branched alkyl group having 1 to 50 carbon atoms. Each hydrogen atom of a C₁₋₂₀ alkyl group, a C₈₋₁₈ alkyl group and C₁₋₅₀ alkyl group may be replaced by a substituent. In each case the alkyl group may be present at the end of a molecule or two moieties of a molecule may be linked by the alkyl group. Optionally, a C₁₋₂₀ alkyl or C₁₋₅₀ alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆ alkenyl group, then an example for such C₂₋₆ alkenyl is —CH═CH—. Each hydrogen atom of a C₂₋₆ alkenyl group may be replaced by a substituent as defined below. Optionally, a C₂₋₆ alkenyl may be interrupted by one or more moieties as defined below.

Accordingly, as used herein, the term “C₂₋₂₀ alkenyl” alone or in combination means a straight-chain or branched hydrocarbon residue comprising at least one carbon-carbon double bond having 2 to 20 carbon atoms. The term “C₂₋₅₀ alkenyl” alone or in combination means a straight-chain or branched hydrocarbon residue comprising at least one carbon-carbon double bond having 2 to 50 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the alkenyl group, then an example is e.g. —CH═CH—. Each hydrogen atom of a C₂₋₂₀ alkenyl or C₂₋₅₀ alkenyl group may be replaced by a substituent as defined below. Optionally, a C₂₋₂₀ alkenyl or C₂₋₅₀ alkenyl may be interrupted by one or more moieties as defined below.

As used herein, the term “C₂₋₆ alkynyl” alone or in combination means straight-chain or branched hydrocarbon residue comprising at least one carbon-carbon triple bond having 2 to 6 carbon atoms. If present at the end of a molecule, examples are —C≡CH, —CH₂—C≡CH, CH₂—CH₂—C≡CH and CH₂—C≡C—CH₃. When two moieties of a molecule are linked by the alkynyl group, then an example is: —C≡C—. Each hydrogen atom of a C₂₋₆ alkynyl group may be replaced by a substituent as defined below. Optionally, one or more double bond(s) may occur. Optionally, a C₂₋₆ alkynyl may be interrupted by one or more moieties as defined below.

Accordingly, as used herein, the term “C₂₋₂₀ alkynyl” alone or in combination means a straight-chain or branched hydrocarbon residue comprising at least one carbon-carbon triple bond having 2 to 20 carbon atoms and “C₂₋₅₀ alkynyl” alone or in combination means a straight-chain or branched hydrocarbon residue comprising at least one carbon-carbon triple bond having 2 to 50 carbon atoms. If present at the end of a molecule, examples are —C≡CH, —CH₂—C≡CH, CH₂—CH₂—C≡CH and CH₂—C≡C—CH₃. When two moieties of a molecule are linked by the alkynyl group, then an example is —C≡C—. Each hydrogen atom of a C₂₋₂₀ alkynyl or C₂₋₅₀ alkynyl group may be replaced by a substituent as defined below. Optionally, one or more double bond(s) may occur. Optionally, a C₂₋₂₀ alkynyl or C₂₋₅₀ alkynyl may be interrupted by one or more moieties as defined below.

As mentioned above, a C₁₋₄ alkyl, C₁₋₆ alkyl, C₁₋₂₀ alkyl, C₁₋₅₀ alkyl, C₂₋₆ alkenyl, C₂₋₂₀ alkenyl, C₂₋₅₀ alkenyl, C₂₋₆ alkynyl, C₂₋₂₀ alkynyl or C₂₋₅₀ alkynyl may optionally be interrupted by one or more of the following moieties:

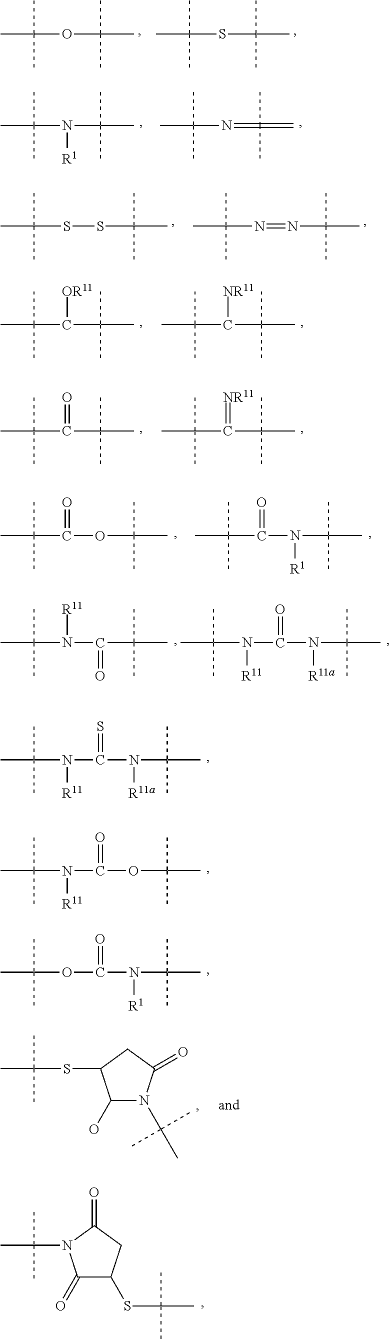

wherein
dashed lines indicate attachment to the remainder of the moiety or reagent, and
R¹¹ and R^{11a} are independently of each other selected from H and methyl, ethyl propyl, butyl, pentyl, hexyl.

As used herein, the terms “C₃₋₈ cycloalkyl” or “C₃₋₈ cycloalkyl ring” means a cyclic alkyl chain having 3 to 8 carbon atoms, which may be saturated or unsaturated, e.g. cyclopropyl, cyclobutyl, cyclopentyl, cyclohexyl, cyclohexenyl, cycloheptyl, cyclooctyl. Each hydrogen atom of a cycloalkyl carbon may be replaced by a substituent as defined below. The term “C₃₋₈cycloalkyl” or “C₃₋₈ cycloalkyl ring” also includes bridged bicycles like norbonane or norbonene. Accordingly, “C₃₋₅ cycloalkyl” means a cycloalkyl having 3 to 5 carbon atoms and C₃₋₁₀ cycloalkyl having 3 to 10 carbon atoms.

Accordingly, as used herein, the term “C₃₋₁₀ cycloalkyl” means a carbocyclic ring system having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl, cyclodecyl. The term “C₃₋₁₀ cycloalkyl” also includes at least partially saturated carbomono- and -bicycles.

As used herein, the term “halogen” means fluoro, chloro, bromo or iodo. Particularly preferred is fluoro or chloro.

As used herein, the term “4- to 7-membered heterocyclyl” or “4- to 7-membered heterocycle” means a ring with 4, 5, 6 or 7 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for 4- to 7-membered heterocycles include but are not limited to azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 4- to 7-membered heterocyclyl or 4- to 7-membered heterocyclic group may be replaced by a substituent as defined below.

As used herein, the term “8- to 11-membered heterobicyclyl” or “8- to 11-membered heterobicycle” means a heterocyclic system of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for a 8- to 1-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent as defined below.

As used herein, the term “interrupted” means that between two carbon atoms or at the end of a carbon chain between the respective carbon atom and the hydrogen atom one or more atom(s) are inserted.

As used herein, the term “prodrug” means a biologically active moiety connected to a specialized non-toxic protective group through a reversible linker to alter or to eliminate undesirable properties in the parent molecule. This also includes the enhancement of desirable properties in the drug and the suppression of undesirable properties. Prodrugs are converted to the parent molecule by biotransformation.

As used herein, the term “biotransformation” refers to the chemical conversion of substances, such as prodrugs, by living organisms or enzyme preparations.

As used herein, the term “carrier-linked prodrug” means a prodrug that comprises a biologically active moiety that is covalently conjugated through a reversible linkage to a carrier moiety and which carrier moiety produces improved physicochemical or pharmacokinetic properties. Upon cleavage of the reversible linkage the biologically active moiety is released as the corresponding drug.

As used herein, the term “hydrogel-linked prodrug” means a carrier-linked prodrug in which the carrier is a hydrogel.

A “reversible linkage/linker” or “biodegradable linkage/linker” is a linkage/linker that is non-enzymatically hydrolytically degradable, i.e. cleavable, under physiological conditions (aqueous buffer at pH 7.4, 37° C.) with a half-life ranging from one hour to six months.

In contrast, a “permanent linkage/linker” or “stable linkage/linker” is a linkage/linker that is non-enzymatically hydrolytically degradable under physiological conditions (aqueous buffer at pH 7.4, 37° C.) with half-lives of more than six months.

As used herein, the term “pharmaceutical composition” means one or more active ingredients, and one or more inert ingredients, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the carrier-linked prodrug of the present invention and one or more pharmaceutically acceptable excipient(s).

As used herein, the term “excipient” refers to a diluent, adjuvant, or vehicle with which the therapeutic is administered. Such pharmaceutical excipient can be sterile liquids, such as water and oils, including those of petroleum, animal, vegetable or synthetic origin, including but not limited to peanut oil, soybean oil, mineral oil, sesame oil and the like. Water is a preferred excipient when the pharmaceutical composition is administered orally. Saline and aqueous dextrose are preferred excipients when the pharmaceutical composition is administered intravenously. Saline solutions and aqueous dextrose and glycerol solutions are preferably employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, water, ethanol and the like. The pharmaceutical composition, if desired, can also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MES (2-(N-morpholino)ethanesulfonic acid), or can contain detergents, like Tween, poloxamers, poloxamines, CHAPS, Igepal, or amino acids like, for example, glycine, lysine, or histidine. These pharmaceutical compositions can take the form of solutions, suspensions, emulsions, tablets, pills, capsules, powders, sustained-release formulations and the like. The pharmaceutical composition can be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such compositions will contain a therapeutically effective amount of the drug or biologically active moiety, together with a suitable amount of excipient so as to provide the form for proper administration to the patient. The formulation should suit the mode of administration.

In general the term “comprise” or “comprising” also encompasses “consist of” or “consisting of”.

The term “relaxin” as used in the present invention is described in further detail in the following sections, but broadly relates to agonists of relaxin receptors and variants thereof.

The relaxin receptor agonist may be a peptide, protein or a small molecule, such as the small molecules described by Xiao et al., 2013, Nature Communications 4, Article number 1953. Preferably, the relaxin receptor agonist is a peptide or protein.

The term “relaxin” also includes single chain relaxin and relaxin in which the two chains are connected through either peptidic or non-peptidic linker moieties as well as prorelaxin.

Such relaxin receptor agonist includes relaxin of human origin, but also from other mammals. Preferably, the term “relaxin” refers to relaxin receptor agonist from human.

Preferably, the term “relaxin” refers to peptides having at least 80% homology to human RLN1 (Universal Protein Resource (UniProt) identifier P04808); human RLN2 (UniProt identifier P04090); human RLN3 (UniProt identifier Q8WXF3); human INSL3 (UniProt identifier P51460); human INSL4 (UniProt identifier Q14641); human INSL5 (UniProt identifier Q9Y5Q6) or human INSL6 (UniProt identifier Q9Y581); each in the form of its prepropeptide, propeptide or mature peptide. Even more preferably, such relaxin peptide has at least 85% homology to human RLN1; human RLN2; human RLN3; human INSL3; human INSL4; human INSL5 or human INSL6; each in the form of its prepropeptide, propeptide or mature peptide. Even more preferably, such relaxin peptide has at least 90% homology to human RLN1; human RLN2; human RLN3; human INSL3; human INSL4; human INSL5 or human INSL6; each in the form of its prepropeptide, propeptide or mature peptide. Even more preferably, such relaxin peptide has at least 95% homology to human RLN1; human RLN2; human RLN3; human INSL3; human INSL4; human INSL5 or human INSL6; each in the form of its prepropeptide, propeptide or mature peptide. Even more preferably, such relaxin peptide has at least 98% homology to human RLN1; human RLN2; human RLN3; human INSL3; human INSL4; human INSL5 or human INSL6; each in the form of its prepropeptide, propeptide or mature peptide.

Most preferably, the term “relaxin” refers to human RLN1; human RLN2; human RLN3; human INSL3; human INSL4; human INSL5 or human INSL6; each in the form of its prepropeptide, propeptide or mature peptide. Most preferably, the term “relaxin” refers to to human RLN1; human RLN2; human RLN3; human INSL3; human INSL4; human INSL5 or human INSL6; each in the form of its mature peptide.

Preferred relaxin drug molecules suitable for use in the carrier-linked relaxin prodrugs of the present invention can be glycosylated or non-glycosylated. Methods for their production and use are, for example, described in U.S. Pat. No. 5,075,222; WO91/08285; WO91/17184; AU 9173636; WO92/16221 and WO96/22793. Furthermore, also relaxin moieties covalently conjugated to polymers, such as for example PEG, and/or conjugated to other moieties such as acyl groups, either as stable or reversible conjugates, are suitable for the carrier-linked relaxin prodrugs of the present invention.

Different methods for the production of relaxin are possible. In a first method, relaxin is isolated from human sample material. A second method for the production of relaxin may be via chemical synthesis, such as solid-phase synthesis, or a combination of such chemical synthesis and molecular biology methods. In a third method, the gene encoding relaxin may be cloned into a suitable vector and subsequently transformed into suitable cell types, from which the protein may then be harvested. Numerous combinations of vectors and cell types are known to the person skilled in the art.

As known to the person skilled in the art, it is today routine work to make e.g. minor amino changes in a protein or peptide of interest (here: relaxin) without significantly affecting the activity of the protein or peptide.

The relaxin molecule used for the carrier-linked relaxin prodrugs of the present invention may also include modified forms of relaxin. These include variant peptides in which amino acids have been (1) deleted from (“deletion variants”), (2) inserted into (“insertion variants”), (3) added to the N- and/or C-terminus (“addition variants”), and/or (4) substituted for (“substitution variants”) residues within the amino acid sequence of relaxin.

Further included are variants containing amino acids different from the 20 naturally occurring protein-coding amino acids or variants which comprise chemical modifications at one or more amino acid residues, such as phosphorylation or glycosylation. Also combinations of different variants may be suitable for the carrier-linked relaxin prodrug of the present invention.

A relaxin deletion variant may typically have a deletion ranging from 1 to 10 amino acids, more typically from 1 to 5 amino acids and most typically from 1 to 3 residues. Such deletion variant may contain one continuous deletion, meaning all deleted amino acids are consecutive residues, or the deletion variant may contain more than one deletion wherein the deletions originate from different parts of the protein.

One or more N-terminal, C-terminal and internal intrasequence deletion(s) and combinations thereof may be used. Deletions within the relaxin amino acid sequence may be made in regions of low homology with the sequence of other members of the relaxin family. Deletions within the relaxin amino acid sequence may be made in areas of substantial homology with the sequences of other members of the relaxin family and will be more likely to significantly modify the biological activity.

Relaxin addition variants may include an amino- and/or carboxyl-terminal fusion ranging in length from one residue to one hundred or more residues, preferably, up to 100 amino acid residues, as well as internal intrasequence insertions of single or multiple amino acids residues. Internal additions may range from 1 to 10 amino acid residues, more typically from 1 to 5 amino acid residues and most typically from 1 to 3 amino acid residues.

Additions at the N-terminus of the relaxin peptide include the addition of a methionine or an additional amino acid residue or sequence. It may also include the fusion of a signal sequence and/or other pre-pro sequences to facilitate the secretion from recombinant host cells. Each relaxin peptide or protein may comprise a signal sequence to be recognized and processed, i.e. cleaved by a signal peptidase, by the host cell.

Variants with additions at their N- or C-terminus include chimeric proteins, wherein each comprises the fusion of relaxin with another peptide or protein, such as for example all or part of a constant domain of a heavy or light chain of human immunoglobulin, fragments or full-length elastin-like peptide, XTEN fragments (see for example WO2011/123813A2), PAS fragments (see for example WO2008/155134A1), fragments of proline/alanine random coil polypeptides (see for example WO2011/144756A1), fragments or full-length of serum albumin (preferably human serum albumin) or fragments or full-length albumin-domain antibodies.

Substitution variants of relaxin have at least one amino acid residue exchanged for a different amino acid residue.

Suitable variants also include naturally-occurring allelic variants and variants artificially generated using molecular biology techniques or other forms of manipulation or mutagenesis. Methods for generating substitution variants of proteins are known to the person skilled in the art.

The sequence of relaxin may also be modified such that glycosylation sites are added. An asparagine-linked glycosylation recognistion site comprises a tripeptide sequence which is specifically recognized by appropriate cellular glycosylation enzymes. These tripeptide sequences are either Asn-Xaa-Thr or Asn-Xaa-Ser, where Xaa can be any amino acid other than Pro.

In one preferred embodiment the relaxin moiety of the carrier-linked relaxin prodrug of the present invention is human relaxin-2 (RLN2). Relaxin-2 consists of two chains, A and B, which are connected through two inter-molecular disulfide bonds and wherein the A chain in addition comprises an intra-molecular disulfide bond. The A-chain of RLN2 has the structure of SEQ ID NO: 1:

QLYSALANKCCHVGCTKRSLARFC

The B-chain of RLN2 has the structure of SEQ ID NO:2:

DSWMEEVIKLCGRELVRAQIAICGMSTWS

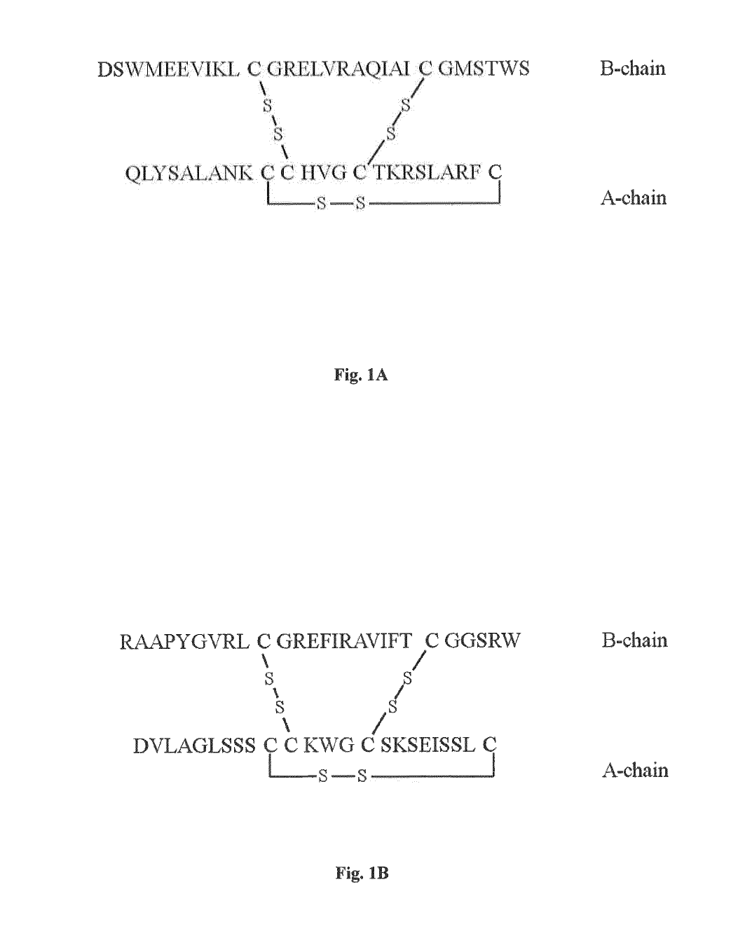

FIG. 1A provides an overview of the A- and B-chain and the location of the two inter- and one intra-molecular disulfide bonds of RLN2.

SEQ ID NO:3 provides the pre-pro-protein sequence of RLN2:

MPRLFFFHLL GVCLLLNQFS RAVADSWMEE VIKLCGRELV
RAQIAICGMS TWSKRSLSQE DAPQTPRPVA EIVPSFINKD
TETINMMSEF VANLPQELKL TLSEMQPALP QLQQHVPVLK
DSSLLFEEFK KLIRNRQSEA ADSSPSELKY LGLDTHSRKK
RQLYSALANK CCHVGCTKRS LARFC

The two inter-molecular disulfide bonds of RLN2 are formed between the thiol moieties of C35/C172 and C47/C185 and the intra-molecular disulfide bond is formed between the thiol moieties of C171/C176. The B-chain of RLN2 includes amino acids 25 to 53 of SEQ ID NO:3 and the A-chain of RLN2 includes amino acids 162 to 185 of SEQ ID NO:3.

In another preferred embodiment the relaxin moiety of the carrier-linked relaxin prodrug of the present invention is human relaxin-3 (RLN3). Relaxin-3 consists of two chains, A and B, which are connected through two inter-molecular disulfide bonds and wherein the A chain in addition comprises an intra-molecular disulfide bond. The A-chain of RLN3 has the structure of SEQ ID NO:4:

DVLAGLSSSCCKWGCSKSEISSLC

The B-chain of RLN3 has the structure of SEQ ID NO:5:

RAAPYGVRLCGREFIRAVIFTCGGSRW

FIG. 1B provides an overview of the A- and B-chain and the location of the two inter- and one intra-molecular disulfide bonds of RLN3.

SEQ ID NO:6 provides the pre-pro-protein sequence of RLN3:

MARYMLLLLL AVWVLTGELW PGAEARAAPY GVRLCGREFI
RAVIFTCGGS RWRRSDILAH EAMGDTFPDA DADEDSLAGE
LDEAMGSSEW LALTKSPQAF YRGRPSWQGT PGVLRGSRDV
LAGLSSSCCK WGCSKSEISS LC

The two inter-molecular disulfide bonds of RLN3 are formed between the thiol moieties of C35/C129 and C47/C142 and the intra-molecular disulfide bond is formed between the thiol moieties of C128/C133. The B-chain of RLN3 includes amino acids 26 to 52 of SEQ ID NO:6 and the A-chain of RLN3 includes amino acids 119 to 142 of SEQ ID NO:6.

Preferably, the relaxin moiety is human relaxin-2 moiety comprising an A-chain of SEQ ID NO:1 and a B-chain of SEQ ID NO:2.

In one embodiment the half-life of the carrier-linked relaxin prodrug of the present invention after subcutaneous injection is at least 20 times longer than the half-life of intravenously administered native relaxin-2 (RLN2). RLN2 refers to the relaxin moiety as shown in FIG. 1A. The half-life of RLN2 can, for example, be measured by administering via subcutaneous injection a defined amount of RLN2, taking blood samples at various time points thereafter and determining the RLN2 concentration in said blood samples from which the half-life of RLN2 can be determined. The half-life of the carrier-linked relaxin prodrug of the present invention is determined accordingly.

Preferably, the carrier of the carrier-linked relaxin prodrug of the present invention comprises C₁₀₋₁₈ alkyl or a polymer. More preferably, the carrier comprises a polymer. Even more preferably, the polymer is selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof. Even more preferably, the carrier comprises PEG or hyaluronic acid and most preferably, the carrier comprises PEG.

In one embodiment the carrier is a water-soluble carrier.

If the carrier is water-soluble, it preferably comprises a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

Even more preferably a water-soluble carrier comprises a polymer selected from PEG, hyaluronic acid, hydroxyethyl starch and polyoxazoline, even more preferably a water-soluble carrier comprises a polymer selected from PEG and hyaluronic acid. In a particularly preferred embodiment the water-soluble polymer is PEG. In an equally preferred embodiment the water-soluble polymer is hyaluronic acid.

If the carrier is water-soluble, it is preferably the carrier described in WO2013/024047 A1, preferably as described in claim 1 therein, which is hereby incorporated by reference.

If the carrier is water-soluble, it is equally preferred that the carrier has the structure as described in WO2013/024047 A1, preferably as described in claim 1 therein, which is hereby incorporated by reference.

If the carrier is water-soluble, it is equally preferred that the carrier has the structure as described in WO2013/024049 A1, preferably as described in claim 1 therein, which is hereby incorporated by reference.

A preferred water-soluble carrier is a multi-arm PEG derivative as, for instance, detailed in the products list of JenKem Technology, USA (accessed by download from http://www.jenkemusa.com/Pages/PEGProducts.aspx on Oct. 15, 2014), such as a 4-arm-PEG derivative, in particular a 4-arm-PEG comprising a pentaerythritol core, an 8-arm-PEG derivative comprising a hexaglycerin core, and an 8-arm-PEG derivative comprising a tripentaerythritol core. More preferably, the carrier comprises a moiety selected from:

a 4-arm PEG Amine comprising a pentaerythritol core:

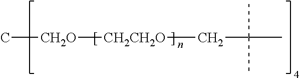

with n ranging from 20 to 500;
an 8-arm PEG Amine comprising a hexaglycerin core:

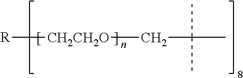

with n ranging from 20 to 500; and
R=hexaglycerin or tripentaerythritol core structure; and
a 6-arm PEG Amine comprising a sorbitol or dipentaerythritol core:

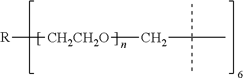

with n ranging from 20 to 500; and
R=comprising a sorbitol or dipentaerythritol core;
and wherein dashed lines indicate attachment to the rest of the carrier-linked relaxin prodrug.

In a preferred embodiment, the molecular weight of such soluble carrier ranges from 1 kDa to 160 kDa, more preferably from 5 kDa to 80 kDa, even more preferably 10 kDa to 40 kDa and most preferably the carrier has a molecular weight of 40 kDa.

In a preferred embodiment the carrier comprises a moiety having following structure:

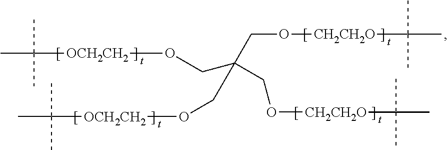

wherein
t ranges from 23 to 3600, preferably from 115 to 1800, even more preferably from 230 to 910 and most preferably t ranges from 900 to 910;
and dashed lines indicate attachment to the rest of the carrier-linked relaxin prodrug.

In another embodiment, the carrier is water-insoluble.

Even more preferably, the carrier is a hydrogel, i.e. the carrier-linked relaxin prodrug is a hydrogel-linked relaxin prodrug.

Preferably, such hydrogel is a shaped article, such as a coating, mesh, stent, nanoparticle or a microparticle. Preferably, the carrier of the carrier-linked relaxin prodrug of the present invention is a hydrogel in the form of a microparticle. More preferably, the hydrogel is a microparticulate bead. Even more preferably, such microparticulate bead has a diameter of 1 to 1000 μm, more preferably of 5 to 500 μm, more preferably of 10 to 250 μm, even more preferably of 15 to 200 μm, even more preferably of 20 to 170 μm, even more preferably of 25 to 150 μm and most preferably of 30 to 100 μm. The afore-mentioned diameters are measured when the hydrogel microparticles are fully hydrated in water at room temperature.

Preferably, the carrier is a PEG-based or hyaluronic acid-based hydrogel. Most preferably, the carrier is a PEG-based hydrogel comprising at least 10% PEG, more preferably at least 15% PEG and most preferably at least 20% PEG.

Suitable hydrogels are known in the art. Preferred hydrogels are those disclosed in WO2006/003014 and WO2011/012715, which are herewith incorporated by reference.

Most preferably, the hydrogel carrier is a hydrogel obtained from a process for the preparation of a hydrogel comprising the steps of:
(a) providing a mixture comprising
(a-i) at least one backbone reagent, wherein the at least one backbone reagent has a molecular weight ranging from 1 to 100 kDa, and comprises at least three functional groups A^{x0}, wherein each A^{x0} is a maleimide, amine (—NH₂ or —NH—), hydroxyl (—OH), thiol (—SH), carboxyl (—COOH) or activated carboxyl (—COY¹, wherein Y¹ is selected from formulas (f-i) to (f-vi):

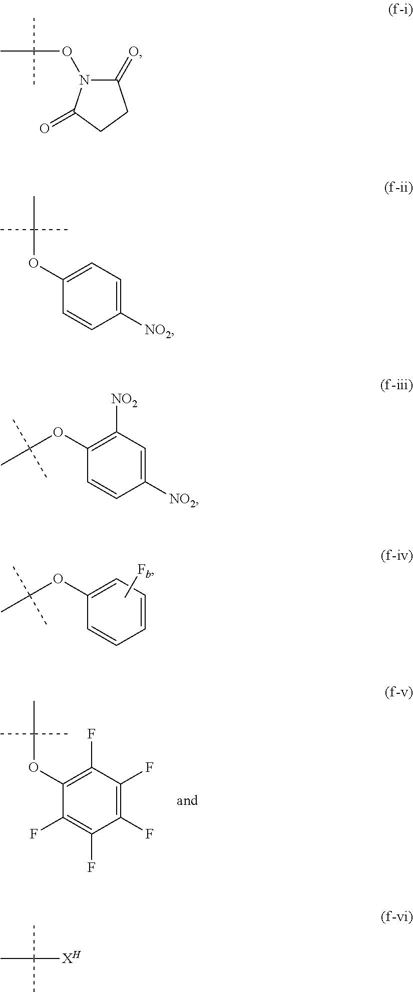

wherein
the dashed lines indicate attachment to the rest of the molecule,
b is 1, 2, 3 or 4,
X^{H} is Cl, Br, I, or F);
(a-ii) at least one crosslinker reagent, wherein the at least one crosslinker reagent has a molecular weight ranging from 0.2 to 40 kDa and comprises at least two functional end groups selected from the group consisting of activated ester groups, activated carbamate groups, activated carbonate groups, activated thiocarbonate groups, amine groups and thiol groups;
in a weight ratio of the at least one backbone reagent to the at least one crosslinker reagent ranging from 1:99 to 99:1 and wherein the molar ratio of A^{x0} to functional end groups is >1;
(b) polymerizing the mixture of step (a) in a suspension polymerization to a hydrogel.

The mixture of step (a) comprises a first solvent and at least a second solvent. Said first solvent is preferably selected from the group comprising dichloromethane, chloroform, tetrahydrofuran, ethyl acetate, dimethylformamide, acetonitrile, dimethyl sulfoxide, propylene carbonate, N-methylpyrrolidone, methanol, ethanol, isopropanol and water and mixtures thereof.

The at least one backbone reagent and at least one crosslinker reagent are dissolved in the first solvent, i.e. the disperse phase of the suspension polymerization. In one embodiment the backbone reagent and the crosslinker reagent are dissolved separately, i.e. in different containers, using either the same or different solvent and preferably using the same solvent for both reagents. In another embodiment, the backbone reagent and the crosslinker reagent are dissolved together, i.e. in the same container and using the same solvent.

A suitable solvent for the backbone reagent is an organic solvent. Preferably, the solvent is selected from the group consisting of dichloromethane, chloroform, tetrahydrofuran, ethyl acetate, dimethylformamide, acetonitrile, dimethyl sulfoxide, propylene carbonate, N-methylpyrrolidone, methanol, ethanol, isopropanol and water and mixtures thereof. More preferably, the backbone reagent is dissolved in a solvent selected from the group comprising acetonitrile, dimethyl sulfoxide, methanol or mixtures thereof. Most preferably, the backbone reagent is dissolved in dimethylsulfoxide.

In one embodiment the backbone reagent is dissolved in the solvent in a concentration ranging from 1 to 300 mg/ml, more preferably from 5 to 60 mg/ml and most preferably from 10 to 40 mg/ml.

A suitable solvent for the crosslinker reagent is an organic solvent. Preferably, the solvent is selected from the group comprising dichloromethane, chloroform, tetrahydrofuran, ethyl acetate, dimethylformamide, acetonitrile, dimethyl sulfoxide, propylene carbonate, N-methylpyrrolidone, methanol, ethanol, isopropanol, water or mixtures thereof. More preferably, the crosslinker reagent is dissolved in a solvent selected from the group comprising dimethylformamide, acetonitrile, dimethyl sulfoxide, methanol or mixtures thereof. Most preferably, the crosslinker reagent is dissolved in dimethylsulfoxide.

In one embodiment the crosslinker reagent is dissolved in the solvent in a concentration ranging from 5 to 500 mg/ml, more preferably from 25 to 300 mg/ml and most preferably from 50 to 200 mg/ml.

The at least one backbone reagent and the at least one crosslinker reagent are mixed in a weight ratio ranging from 1:99 to 99:1, e.g. in a ratio ranging from 2:98 to 90:10, in a weight ratio ranging from 3:97 to 88:12, in a weight ratio ranging from 3:96 to 85:15, in a weight ratio ranging from 2:98 to 90:10 and in a weight ratio ranging from 5:95 to 80:20; particularly preferred in a weight ratio from 5:95 to 80:20, wherein the first number refers to the backbone reagent and the second number to the crosslinker reagent.

Preferably, the ratios are selected such that the mixture of step (a) comprises a molar excess of amine groups from the backbone reagent compared to the activated functional end groups of the crosslinker reagent. Consequently, the hydrogel resulting from the process has free amine groups which can be used to couple other moieties to the hydrogel, such as spacers, and/or reversible linker moieties L¹.

The at least one second solvent, i.e. the continuous phase of the suspension polymerization, is preferably an organic solvent, more preferably an organic solvent selected from the group comprising linear, branched or cyclic C₅₋₃₀ alkanes; linear, branched or cyclic C₅₋₃₀ alkenes; linear, branched or cyclic C₅₋₃₀ alkynes; linear or cyclic poly(dimethylsiloxanes); aromatic C₆₋₂₀ hydrocarbons; and mixtures thereof. Even more preferably, the at least second solvent is selected from the group comprising linear, branched or cyclic C₅₋₁₆ alkanes; toluene; xylene; mesitylene; hexamethyldisiloxane; or mixtures thereof. Most preferably, the at least second solvent selected from the group comprising linear C₇₋₁₁ alkanes, such as heptane, octane, nonane, decane and undecane.

Preferably, the mixture of step (a) further comprises a detergent. Preferred detergents are Cithrol DPHS, Hypermer 70A, Hypermer B246, Hypermer 1599A, Hypermer 2296, and Hypermer 1083.

Preferably, the detergent has a concentration of 0.1 g to 100 g per 1 L total mixture, i.e. disperse phase and continuous phase together. More preferably, the detergent has a concentration of 0.5 g to 10 g per 1 L total mixture, and most preferably, the detergent has a concentration of 0.5 g to 5 g per 1 L total mixture.

Preferably, the mixture of step (a) is an emulsion.

The polymerization in step (b) is initiated by adding a base. Preferably, the base is a non-nucleophilic base soluble in alkanes, more preferably the base is selected from N,N,N′,N′-tetramethylethylene diamine (TMEDA), 1,4-dimethylpiperazine, 4-methylmorpholine, 4-ethylmorpholine, 1,4-diazabicyclo[2.2.2]octane, 1,1,4,7,10,10-hexamethyltriethylenetetramine, 1,4,7-trimethyl-1,4,7-triazacyclononane, tris[2-(dimethylamino)ethyl]amine, triethylamine, DIPEA, trimethylamine, N,N-dimethylethylamine, N,N,N′,N′-tetramethyl-1,6-hexanediamine, N,N,N′,N″,N″-pentamethyldiethylenetriamine, 1,8-diazabicyclo[5.4.0]undec-7-ene, 1,5-diazabicyclo[4.3.0]non-5-ene, and hexamethylenetetramine. Even more preferably, the base is selected from TMEDA, 1,4-dimethylpiperazine, 4-methylmorpholine, 4-ethylmorpholine, 1,4-diazabicyclo[2.2.2]octane, 1,1,4,7,10,10-hexamethyltriethylenetetramine, 1,4,7-trimethyl-1,4,7-triazacyclononane, tris[2-(dimethylamino)ethyl]amine, 1,8-diazabicyclo[5.4.0]undec-7-ene, 1,5-diazabicyclo[4.3.0]non-5-ene, and hexamethylenetetramine. Most preferably, the base is TMEDA.

The base is added to the mixture of step (a) in an amount of 1 to 500 equivalents per activated functional end group in the mixture, preferably in an amount of 5 to 50 equivalents, more preferably in an amount of 5 to 25 equivalents and most preferably in an amount of 10 equivalents.

In process step (b), the polymerization of the hydrogel of the present invention is a condensation reaction, which preferably occurs under continuous stirring of the mixture of step (a). Preferably, the tip speed (tip speed=π×stirrer rotational speed×stirrer diameter) ranges from 0.2 to 10 meter per second (m/s), more preferably from 0.5 to 4 m/s and most preferably from 1 to 2 m/s.

In a preferred embodiment of step (b), the polymerization reaction is carried out in a cylindrical vessel equipped with baffles. The diameter to height ratio of the vessel may range from 4:1 to 1:2, more preferably the diameter to height ratio of the vessel ranges from 2:1 to 1:1.

Preferably, the reaction vessel is equipped with an axial flow stirrer selected from the group comprising pitched blade stirrer, marine type propeller, or Lightnin A-310. More preferably, the stirrer is a pitched blade stirrer.

Step (b) can be performed in a broad temperature range, preferably at a temperature from −10° C. to 100° C., more preferably at a temperature of 0° C. to 80° C., even more preferably at a temperature of 10° C. to 50° C. and most preferably at ambient temperature. “Ambient temperature” refers to the temperature present in a typical laboratory environment and preferably means a temperature ranging from 17 to 25° C.

Preferably, the hydrogel obtained from the polymerization is a shaped article, such as a coating, mesh, stent, nanoparticle or a microparticle. More preferably, the hydrogel is in the form of microparticular beads having a diameter from 1 to 500 micrometer, more preferably with a diameter from 10 to 300 micrometer, even more preferably with a diameter from 20 and 150 micrometer and most preferably with a diameter from 30 to 130 micrometer. The afore-mentioned diameters are measured when the hydrogel microparticles are fully hydrated in water.

In one embodiment, the process for the preparation of a hydrogel further comprises the step of:

(c) working-up the hydrogel.

Step (c) comprises one or more of the following step(s):

(c1) removing excess liquid from the polymerization reaction,
(c2) washing the hydrogel to remove solvents used during polymerization,
(c3) transferring the hydrogel into a buffer solution,
(c4) size fractionating/sieving of the hydrogel,
(c5) transferring the hydrogel into a container,
(c6) drying the hydrogel,
(c7) transferring the hydrogel into a specific solvent suitable for sterilization, and
(c8) sterilizing the hydrogel, preferably by gamma radiation

Preferably, step (c) comprises all of the following steps

(c1) removing excess liquid from the polymerization reaction,
(c2) washing the hydrogel to remove solvents used during polymerization,
(c3) transferring the hydrogel into a buffer solution,
(c4) size fractionating/sieving of the hydrogel,
(c5) transferring the hydrogel into a container,
(c7) transferring the hydrogel into a specific solvent suitable for sterilization, and
(c8) sterilizing the hydrogel, preferably by gamma radiation.

The at least one backbone reagent has a molecular weight ranging from 1 to 100 kDa, preferably from 2 to 50 kDa, more preferably from 5 and 30 kDa, even more preferably from 5 to 25 kDa and most preferably from 5 to 15 kDa.

Preferably, the backbone reagent is PEG-based comprising at least 10% PEG, more preferably comprising at least 20% PEG, even more preferably comprising at least 30% PEG and most preferably comprising at least 40% PEG.

In one embodiment the backbone reagent of step (a-i) is present in the form of its acidic salt, preferably in the form of an acid addition salt. Suitable acid addition salts are formed from acids which form non-toxic salts. Examples include but are not limited to the acetate, aspartate, benzoate, besylate, bicarbonate, carbonate, bisulphate, sulphate, borate, camsylate, citrate, edisylate, esylate, formate, fumarate, gluceptate, gluconate, glucuronate, hexafluorophosphate, hibenzate, hydrochloride, hydrobromide, hydroiodide, isethionate, lactate, malate, maleate, malonate, mesylate, methylsulphate, naphthylate, nicotinate, nitrate, orotate, oxalate, palmitate, pamoate, phosphate, hydrogen phosphate, dihydrogen phosphate, sacharate, stearate, succinate, tartrate and tosylate. Particularly preferred, the backbone reagent is present in the form of its hydrochloride salt.

In one embodiment, the at least one backbone reagent is selected from the group consisting of
a compound of formula (aI)

B(-(A⁰)ₓ₁-(SP)ₓ₂-A¹-P-A²-Hyp¹)ₓ (aI),
wherein
B is a branching core,
SP is a spacer moiety selected from the group consisting of C₁₋₆ alkyl, C₂₋₆ alkenyl and C₂₋₆ alkynyl,
P is a PEG-based polymeric chain comprising at least 80% PEG, preferably at least 85% PEG, more preferably at least 90% PEG and most preferably at least 95% PEG,
Hyp¹ is a moiety comprising an amine (—NH₂ and/or —NH—) or a polyamine comprising at least two amines (—NH₂ and/or —NH—),
x is an integer from 3 to 16,
x1, x2 are independently of each other 0 or 1, provided that x1 is 0, if x2 is 0,
A⁰, A¹, A² are independently of each other selected from the group consisting of

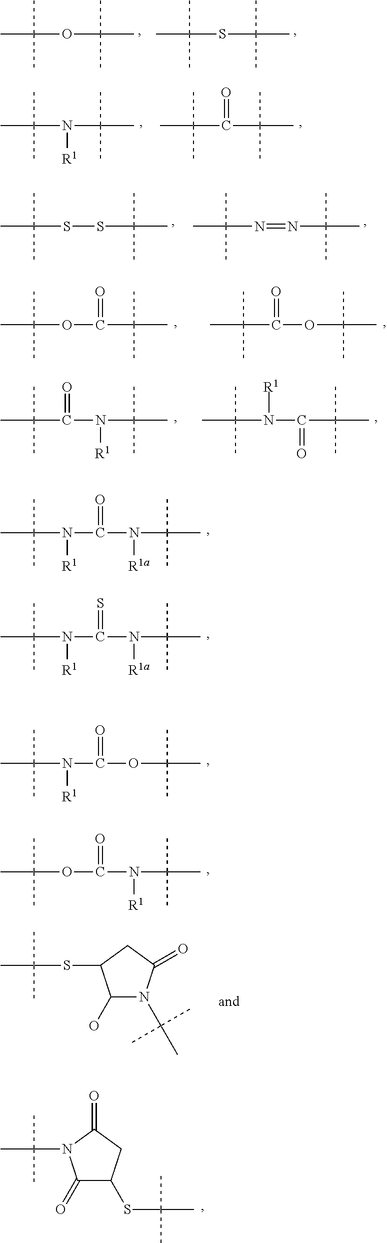

wherein R¹ and R^{1a} are independently of each other selected from H and C₁₋₆ alkyl;
a compound of formula (aII)

Hyp²-A³-P-A⁴-Hyp³ (aII),
wherein
P is defined as above in the compound of formula (aI),
Hyp², Hyp³ are independently of each other a polyamine comprising at least two amines (—NH₂ and/or —NH—), and
A³ and A⁴ are independently selected from the group consisting of

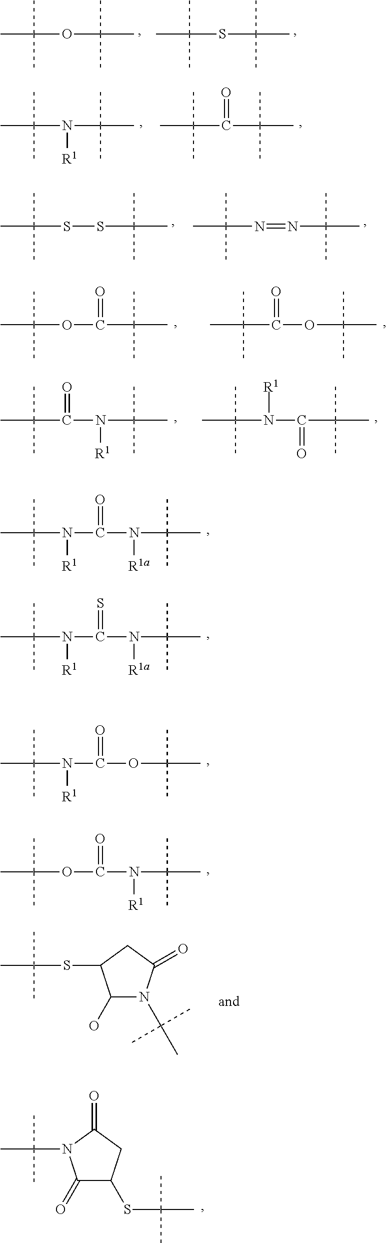

wherein R¹ and R^{1a} are independently of each other selected from H and C₁₋₆ alkyl;
a compound of formula (aIII)

P¹-A⁵-Hyp⁴ (aIII),
wherein
P¹ is a PEG-based polymeric chain comprising at least 80% PEG, preferably at least 85% PEG, more preferably at least 90% PEG and most preferably at least 95% PEG,
Hyp⁴ is a polyamine comprising at least three amines (—NH₂ and/or —NH), and
A⁵ is selected from the group consisting of

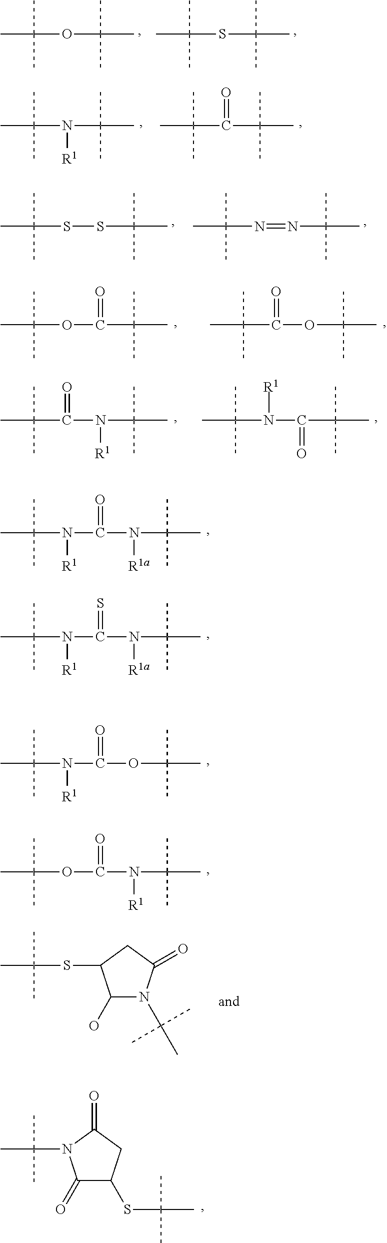

wherein R¹ and R^{1a} are independently of each other selected from H and C₁₋₆ alkyl;
and
a compound of formula (aIV),

T¹-A⁶-Hyp⁵ (aIV),
wherein
Hyp⁵ is a polyamine comprising at least three amines (—NH₂ and/or —NH), and
A⁶ is selected from the group consisting of

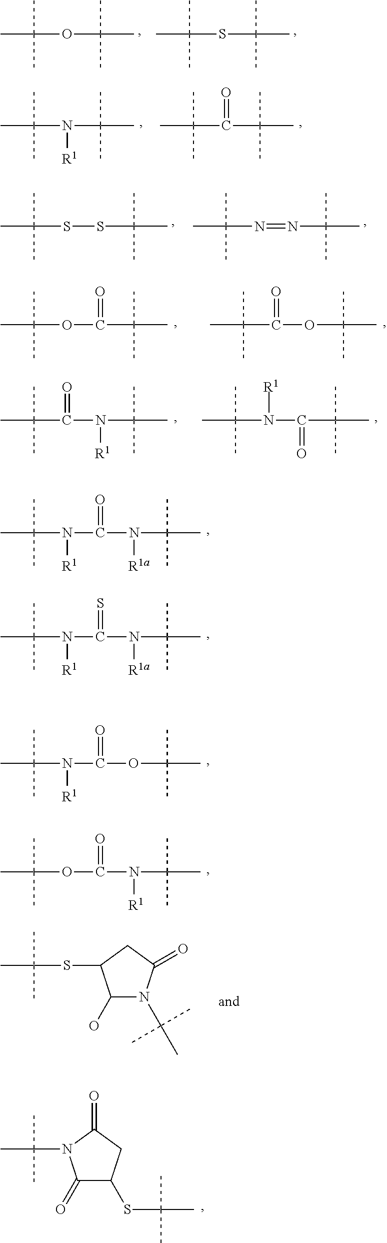

wherein R¹ and R^{1a} are independently of each other selected from H and C₁₋₆ alkyl; and
T¹ is selected from the group consisting of C₁₋₅₀ alkyl, C₂₋₅₀ alkenyl or C₂₋₅₀ alkynyl, which fragment is optionally interrupted by one or more group(s) selected from —NH—, —N(C₁₋₄ alkyl)-, —O—, —S—, —C(O)—, —C(O)NH—, —C(O)N(C₁₋₄ alkyl)-, —O—C(O)—, —S(O)—, —S(O)₂—, 4- to 7-membered heterocyclyl, phenyl or naphthyl.

In the following sections the term “Hyp^{x}” refers to Hyp¹, Hyp², Hyp³, Hyp⁴ and Hyp⁵ collectively.

Preferably, the backbone reagent is a compound of formula (aI), (aII) or (aIII), more preferably the backbone reagent is a compound of formula (aI) or (aIII), and most preferably the backbone reagent is a compound of formula (aI).

In a preferred embodiment, in a compound of formula (aI), x is 4, 6 or 8. Preferably, in a compound of formula (aI) x is 4 or 8, most preferably, x is 4.

In a preferred embodiment in the compounds of the formulas (aI) to (aIV), A⁰, A¹, A², A³, A⁴, A⁵ and A⁶ are selected from the group comprising

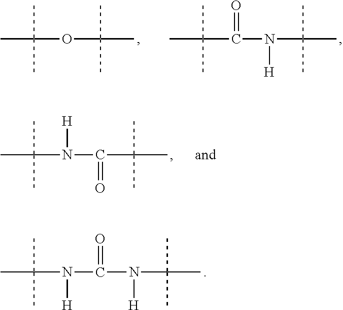

Preferably, in a compound of formula (aI), A⁰ is

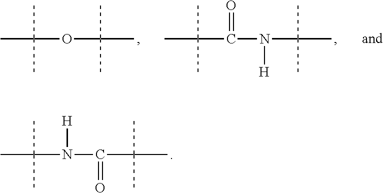

Preferably, in a compound of formula (aI), A¹ is

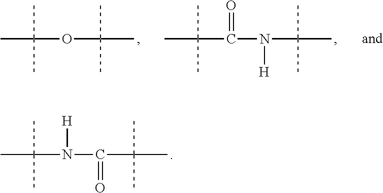

Preferably, in a compound of formula (aI), A² is

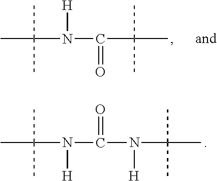

Preferably, in a compound of formula (aII), A³

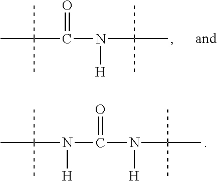

### and A⁴ is

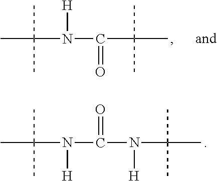

Preferably, in a compound of formula (aIII), A⁵ is

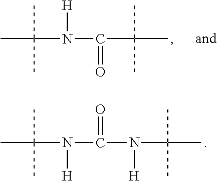

Preferably, in a compound of formula (aIV), A⁶ is

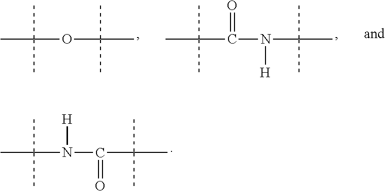

Preferably, in a compound of formula (aIV), T¹ is selected from H and C₁₋₆ alkyl.

In one embodiment, in a compound of formula (aI), the branching core B is selected from the following structures:

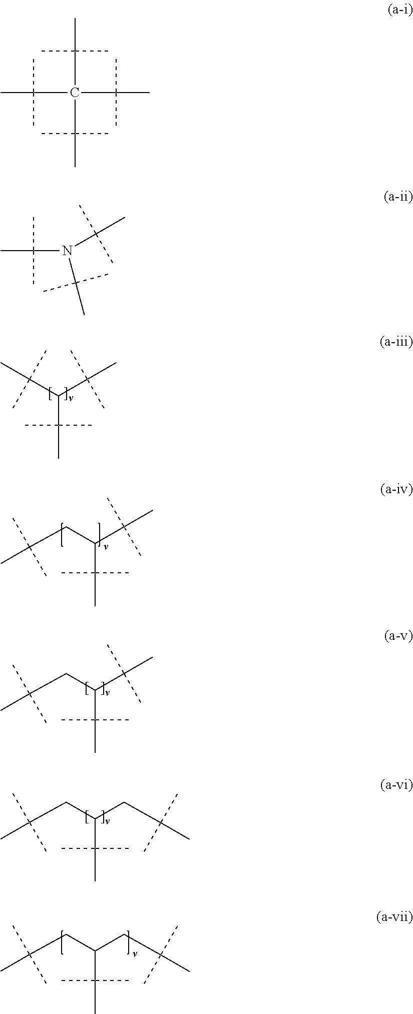

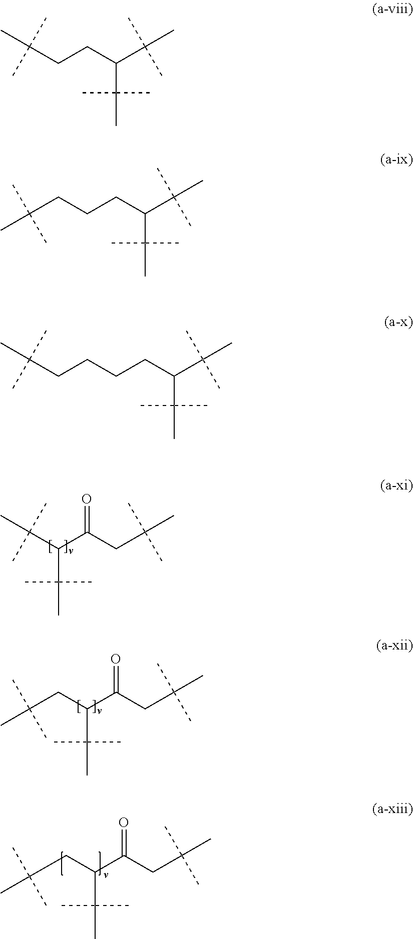

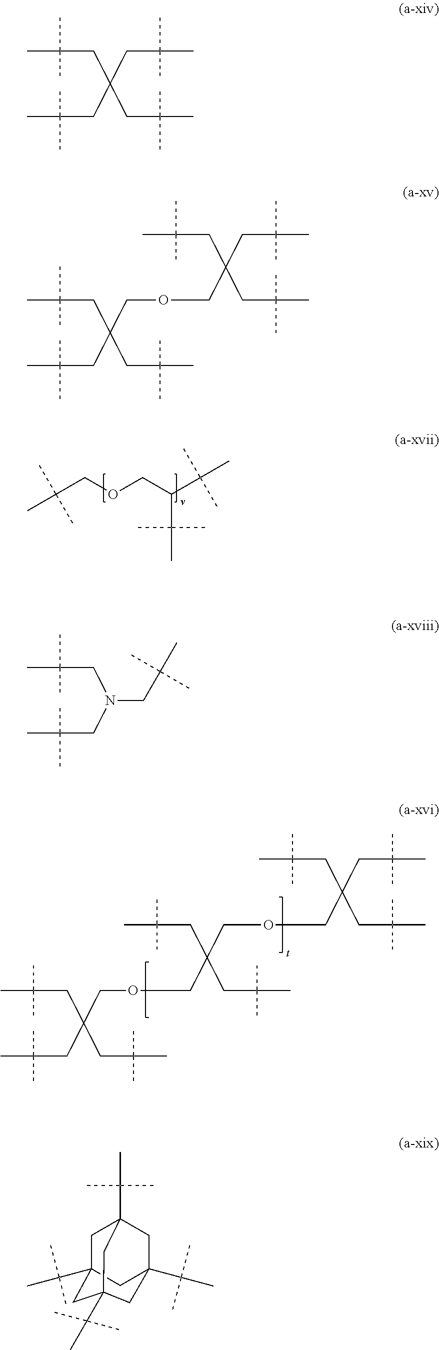

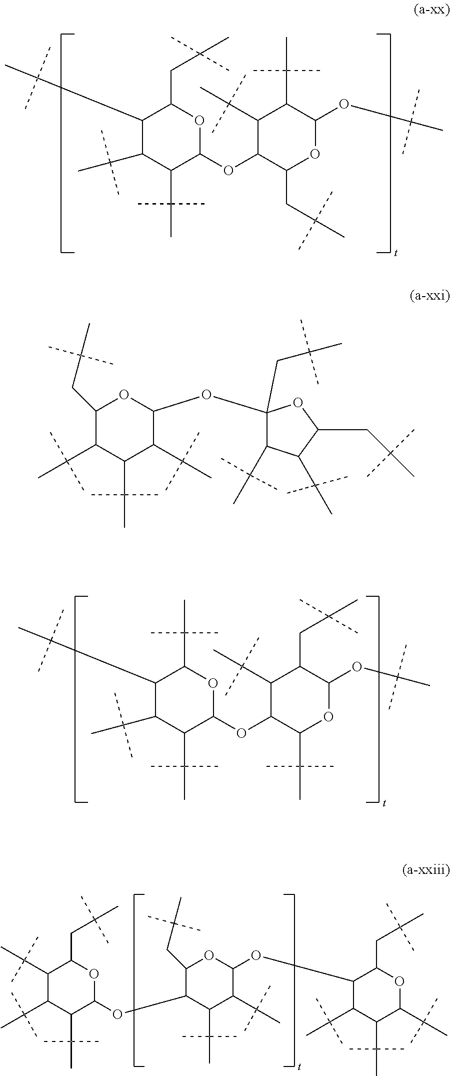

wherein
dashed lines indicate attachment to A⁰ or, if x1 and x2 are both 0, to A¹,
t is 1 or 2; preferably t is 1,
v is 1, 2, 3, 4, 5, 6, 7, 8, 9, 10, 11, 12, 13 or 14; preferably, v is 2, 3, 4, 5, 6; more preferably, v is 2, 4 or 6; most preferably, v is 2.

In a preferred embodiment, B has a structure of formula (a-i), (a-ii), (a-iii), (a-iv), (a-v), (a-vi), (a-vii), (a-viii), (a-ix), (a-x), (a-xiv), (a-xv) or (a-xvi). More preferably, B has a structure of formula (a-iii), (a-iv), (a-v), (a-vi), (a-vii), (a-viii), (a-ix), (a-x) or (a-iv). Most preferably, B has a structure of formula (a-xiv).

A preferred embodiment is a combination of B and A⁰, or, if x1 and x2 are both 0 a preferred combination of B and A¹, which is selected from the following structures:

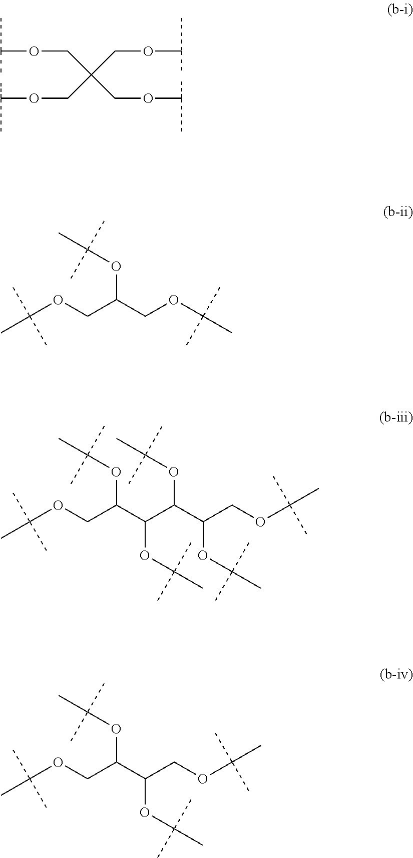

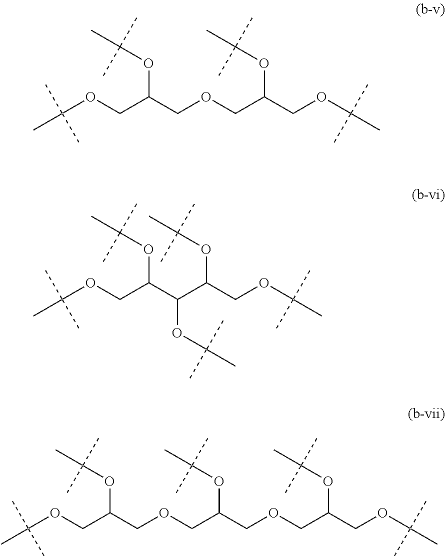

wherein
dashed lines indicate attachment to SP or, if x1 and x2 are both 0, to P.

More preferably, the combination of B and A⁰ or, if x1 and x2 are both 0, the combination of B and A¹, has a structure of formula of formula (b-i), (b-iv), (b-vi) or (b-viii) and most preferably has a structure of formula of formula (b-i).

In one embodiment, x1 and x2 of formula (aI) are 0.

In one embodiment, the PEG-based polymeric chain P has a molecular weight from 0.3 kDa to 40 kDa; e.g. from 0.4 to 35 kDa, from 0.6 to 38 kDA, from 0.8 to 30 kDa, from 1 to 25 kDa, from 1 to 15 kDa or from 1 to 10 kDa. Most preferably P has a molecular weight from 1 to 10 kDa.

In one embodiment, the PEG-based polymeric chain P¹ has a molecular weight from 0.3 kDa to 40 kDa; e.g. from 0.4 to 35 kDa, from 0.6 to 38 kDA, from 0.8 to 30 kDa, from 1 to 25 kDa, from 1 to 15 kDa or from 1 to 10 kDa. Most preferably P¹ has a molecular weight from 1 to 10 kDa.

In one embodiment, in the compounds of formulas (aI) or (aII), P has the structure of formula (c-i):

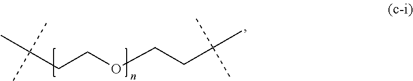

wherein n ranges from 6 to 900, more preferably n ranges from 20 to 700 and most preferably n ranges from 20 to 250.

In one embodiment, in the compounds of formulas (aIII), P¹ has the structure of formula (c-ii):

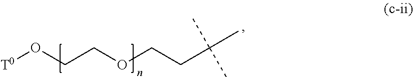

wherein
n ranges from 6 to 900, more preferably n ranges from 20 to 700 and most preferably n ranges from 20 to 250;
T⁰ is selected from the group comprising C₁₋₆ alkyl, C₂₋₆ alkenyl and C₂₋₆ alkynyl, which is optionally interrupted by one or more group(s) selected from —NH—, —N(C₁₋₄ alkyl)-, —O—, —S—, —C(O)—, —C(O)NH—, —C(O)N(C₁₋₄alkyl)-, —O—C(O)—, —S(O)— or —S(O)₂—.

In one embodiment, in the compounds of formulas (aI) to (aIV), the moiety Hyp^{x} is a polyamine and preferably comprises in bound form and, where applicable, in R- and/or S-configuration a moiety of the formulas (d-i), (d-ii), (d-iii) and/or (d-vi):

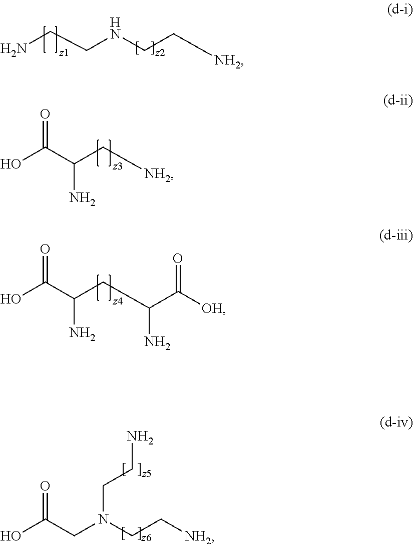

wherein
z1, z2, z3, z4, z5, z6 are independently of each other 1, 2, 3, 4, 5, 6, 7 or 8.

More preferably, Hyp^{x} comprises in bound form and in R- and/or S-configuration lysine, ornithine, diaminoproprionic acid and/or diaminobutyric acid. Most preferably, Hyp^{x} comprises in bound form and in R- and/or S-configuration lysine.

Hyp^{x} has a molecular weight from 40 Da to 30 kDa, preferably from 0.3 kDa to 25 kDa, more preferably from 0.5 kDa to 20 kDa, even more preferably from 1 kDa to 20 kDa and most preferably from 2 kDa to 15 kDa.

Hyp^{x} is preferably selected from the group consisting of
a moiety of formula (e-i)

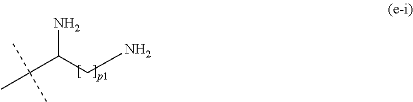

wherein
p1 is an integer from 1 to 5, preferably p1 is 4, and
the dashed line indicates attachment to A² if the backbone reagent has a structure of formula (aI) and to A³ or A⁴ if the backbone reagent has the structure of formula (aII);
a moiety of formula (e-ii)

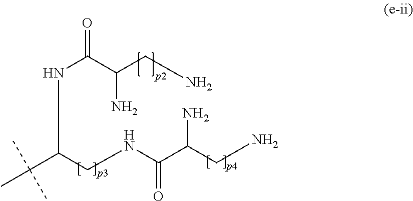

wherein
p2, p3 and p4 are identical or different and each is independently of the others an integer from 1 to 5, preferably p2, p3 and p4 are 4, and
the dashed line indicates attachment to A² if the backbone reagent has a structure of formula (aI), to A³ or A⁴ if the backbone reagent has a structure of formula (aII), to A⁵ if the backbone reagent has a structure of formula (aIII) and to A⁶ if the backbone reagent has a structure of formula (aIV);
a moiety of formula (e-iii)

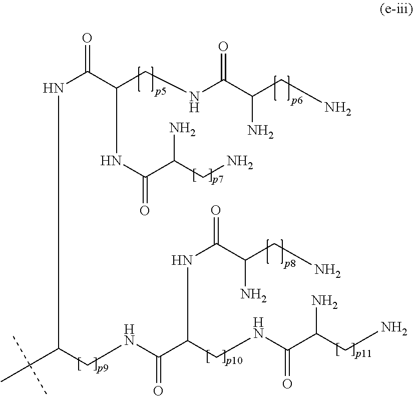

wherein
p5 to p11 are identical or different and each is independently of the others an integer from 1 to 5, preferably p5 to p11 are 4, and
the dashed line indicates attachment to A² if the backbone reagent is of formula (aI), to A³ or A⁴ if the backbone reagent is of formula (aII), to A⁵ if the backbone reagent is of formula (aIII) and to A⁶ if the backbone reagent is of formula (aIV);
a moiety of formula (e-iv)

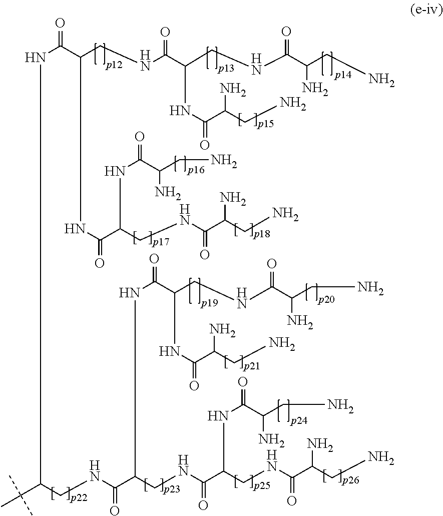

wherein
p12 to p26 are identical or different and each is independently of the others an integer from 1 to 5, preferably p12 to p26 are 4, and
the dashed line indicates attachment to A² if the backbone reagent has a structure of formula (aI), to A³ or A⁴ if the backbone reagent has a structure of formula (aII), to A⁵ if the backbone reagent has a structure of formula (aIII) and to A⁶ if the backbone reagent has a structure of formula (aIV);
a moiety of formula (e-v)

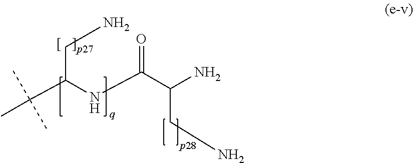

wherein
p27 and p28 are identical or different and each is independently of the other an integer from 1 to 5, preferably p27 and p28 are 4,
q is an integer from 1 to 8, preferably q is 2 or 6 and most preferably 1 is 6, and
the dashed line indicates attachment to A² if the backbone reagent has a structure of formula (aI), to A³ or A⁴ if the backbone reagent has a structure of formula (aII), to A⁵ if the backbone reagent has a structure of formula (aIII) and to A⁶ if the backbone reagent has a structure of formula (aIV);
a moiety of formula (e-vi)

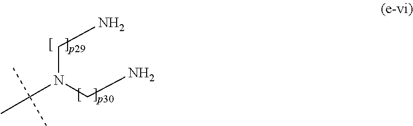

wherein
p29 and p30 are identical or different and each is independently of the other an integer from 2 to 5, preferably p29 and p30 are 3, and
the dashed line indicates attachment to A² if the backbone reagent has the structure of formula (aI), to A³ or A⁴ if the backbone reagent has the structure of formula (aII), to A⁵ if the backbone reagent has the structure of formula (aIII) and to A⁶ if the backbone reagent has the structure of formula (aIV);
a moiety of formula (e-vii)

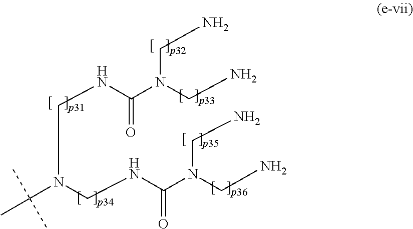

wherein
p31 to p36 are identical or different and each is independently of the others an integer from 2 to 5, preferably p31 to p36 are 3, and
the dashed line indicates attachment to A² if the backbone reagent has a structure of formula (aI), to A³ or A⁴ if the backbone reagent has a structure of formula (aII), to A⁵ if the backbone reagent has a structure of formula (aIII) and to A⁶ if the backbone reagent has a structure of formula (aIV);
a moiety of formula (e-viii)

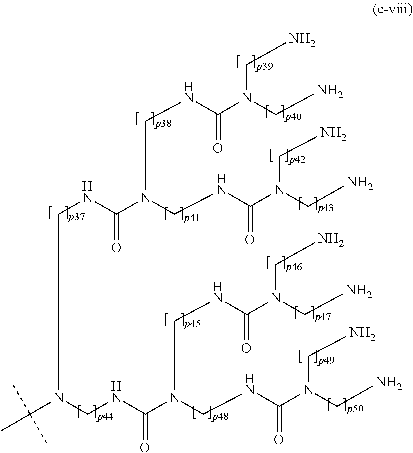

wherein
p37 to p50 are identical or different and each is independently of the others an integer from 2 to 5, preferably p37 to p50 are 3, and
the dashed line indicates attachment to A² if the backbone reagent has a structure of formula (aI), to A³ or A⁴ if the backbone reagent has a structure of formula (aII), to A⁵ if the backbone reagent has a structure of formula (aIII) and to A⁶ if the backbone reagent has a structure of formula (aIV); and
a moiety of formula (e-ix):

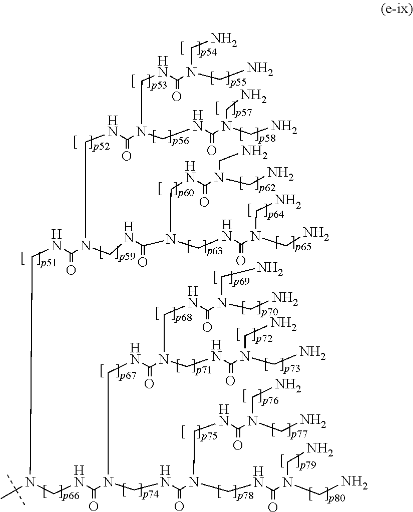

wherein
p51 to p80 are identical or different and each is independently of the others an integer from 2 to 5, preferably p51 to p80 are 3, and
the dashed line indicates attachment to A² if the backbone reagent has a structure of formula (aI), to A³ or A⁴ if the backbone reagent has a structure of formula (aII), to A⁵ if the backbone reagent has a structure of formula (aIII) and to A⁶ if the backbone reagent has a structure of formula (aIV); and
   wherein the moieties (e-i) to (e-v) may at each chiral center be in either R- or S-configuration, preferably, all chiral centers of a moiety (e-i) to (e-v) are in the same configuration.

Preferably, Hyp^{x} is has a structure of formulas (e-i), (e-ii), (e-iii), (e-iv), (e-vi), (e-vii), (e-viii) or (e-ix). More preferably, Hyp^{x} has a structure of formulas (e-ii), (e-iii), (e-iv), (e-vii), (e-viii) or (e-ix), even more preferably Hyp^{x} has a structure of formulas (e-ii), (e-iii), (e-vii) or (e-viii) and most preferably Hyp^{x} has the structure of formula (e-iii).

If the backbone reagent has a structure of formula (aI), a preferred moiety -A²-Hyp¹ is a moiety of the formula

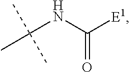

wherein
the dashed line indicates attachment to P; and
E¹ is selected from formulas (e-i) to (e-ix).

If the backbone reagent has a structure of formula (aII) a preferred moiety Hyp²-A³-is a moiety of the formula

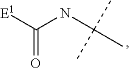

wherein
the dashed line indicates attachment to P; and
E¹ is selected from formulas (e-i) to (e-ix);
   and a preferred moiety -A⁴-Hyp³ is a moiety of the formula

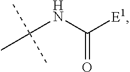

wherein
the dashed line indicates attachment to P; and
E¹ is selected from formulas (e-i) to (e-ix).

If the backbone reagent has a structure of formula (aIII), a preferred moiety -A⁵-Hyp⁴ is a moiety of the formula

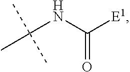

wherein
the dashed line indicates attachment to P¹; and
E¹ is selected from formulas (e-i) to (e-ix).

More preferably, the backbone reagent has a structure of formula (aI) and B is has a structure of formula (a-xiv).

Even more preferably, the backbone reagent has the structure of formula (aI), B has the structure of formula (a-xiv), x1 and x2 are 0, and A¹ is —O—.

Even more preferably, the backbone reagent has the structure of formula (aI), B has the structure of formula (a-xiv), A¹ is —O—, and P has a structure of formula (c-i).

Even more preferably, the backbone reagent is formula (aI), B is of formula (a-xiv), x1 and x2 are 0, A¹ is —O—, P is of formula (c-i), A² is —NH—(C═O)— and Hyp¹ is of formula (e-iii).

Most preferably, the backbone reagent has the following formula:

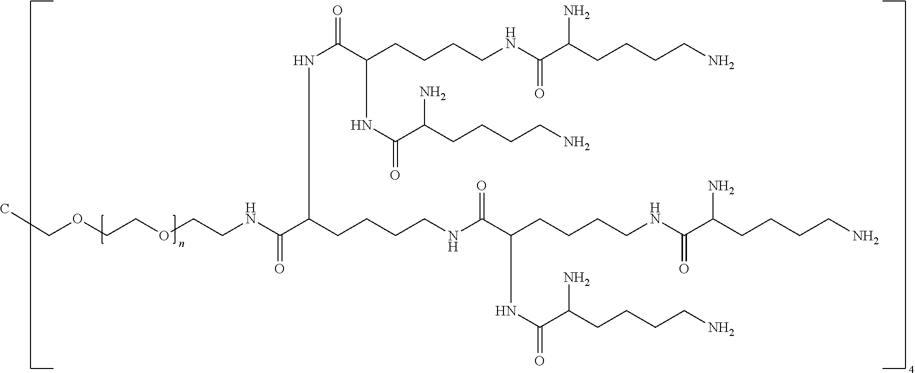

wherein
n ranges from 10 to 40, preferably from 10 to 30, more preferably from 20 to 30 and most preferably n is 28.

SP is a spacer moiety selected from the group comprising C₁₋₆ alkyl, C₂₋₆ alkenyl and C₂₋₆ alkynyl, preferably SP is —CH₂—, —CH₂—CH₂—, —CH(CH₃)—, —CH₂—CH₂—CH₂—, —CH(C₂H₅)—, —C(CH₃)₂—, —CH═CH— or —CH═CH—, most preferably SP is —CH₂—, —CH₂—CH₂— or —CH═CH—.

The at least one crosslinker reagent of step (a-ii) comprises at least two carbonyloxy groups (—(C═O)—O— or —O—(C═O)—), which are biodegradable linkages. These biodegradable linkages are necessary to render the hydrogel biodegradable. Additionally, the at least one crosslinker reagent comprises at least two activated functional end groups which during the polymerization of step (b) react with the amines of the at least one backbone reagent.

The crosslinker reagent has a molecular weight ranging from 0.5 to 40 kDa, more preferably ranging from 0.75 to 30 kDa, even more preferably ranging from 1 to 20 kDa, even more preferably ranging from 1 to 10 kDa, even more preferably ranging from 1 to 7.5 kDa and most preferably ranging from 2 kDa to 4 kDa.

The crosslinker reagent comprises at least two activated functional end groups selected from the group comprising activated ester groups, activated carbamate groups, activated carbonate groups and activated thiocarbonate groups, which during polymerization react with the amine groups of the backbone reagents, forming amide bonds.

In one preferred embodiment, the crosslinker reagent is a compound of formula (VI):

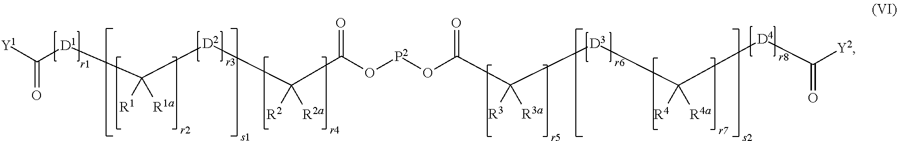

wherein
each D¹, D², D³ and D⁴ are identical or different and each is independently of the others selected from the group comprising —O—, —NR⁵—, —S— and —CR⁶R^{6a}—;
each R¹, R^{1a}, R², R^{2a}, R³, R^{3a}, R⁴, R^{4a}, R⁶ and R^{6a} are identical or different and each is independently of the others selected from the group comprising —H, —OR⁷, —NR⁷R^{7a}, —SR⁷ and C₁₋₆ alkyl; optionally, each of the pair(s) R¹/R², R³/R⁴, R^{1a}/R^{2a}, and R^{3a}/R^{4a} may independently form a chemical bond and/or each of the pairs R¹/R^{1a}, R²/R^{2a}, R³/R^{3a}, R⁴/R^{4a}, R⁶/R^{6a}, R¹/R², R³/R⁴, R^{1a}/R^{2a}, and R^{3a}/R^{4a} are independently of each other joined together with the atom to which they are attached to form a C₃₋₈ cycloalkyl or to form a ring A or are joined together with the atom to which they are attached to form a 4- to 7-membered heterocyclyl or 8- to 1-membered heterobicyclyl or adamantyl;
each R⁵ is independently selected from —H and C₁₋₆ alkyl; optionally, each of the pair(s) R¹/R⁵, R²/R⁵, R³/R⁵, R⁴/R⁵ and R⁵/R⁶ may independently form a chemical bond and/or are joined together with the atom to which they are attached to form a 4- to 7-membered heterocyclyl or 8- to 11-membered heterobicyclyl;
each R⁷, R^{7a} is independently selected from H and C₁₋₆ alkyl;
A is selected from the group consisting of indenyl, indanyl and tetralinyl;
P² is

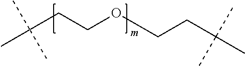

m ranges from 120 to 920, preferably from 120 to 460 and more preferably from 120 to 230;
r1, r2, r7, r8 are independently 0 or 1;
r3, r6 are independently 0, 1, 2, 3, or 4;
r4, r5 are independently 1, 2, 3, 4, 5, 6, 7, 8, 9 or 10;
s1, s2 are independently 1, 2, 3, 4, 5 or 6;
Y¹, Y² are identical or different and each is independently of the other selected from formulas (f-i) to (f-vi):

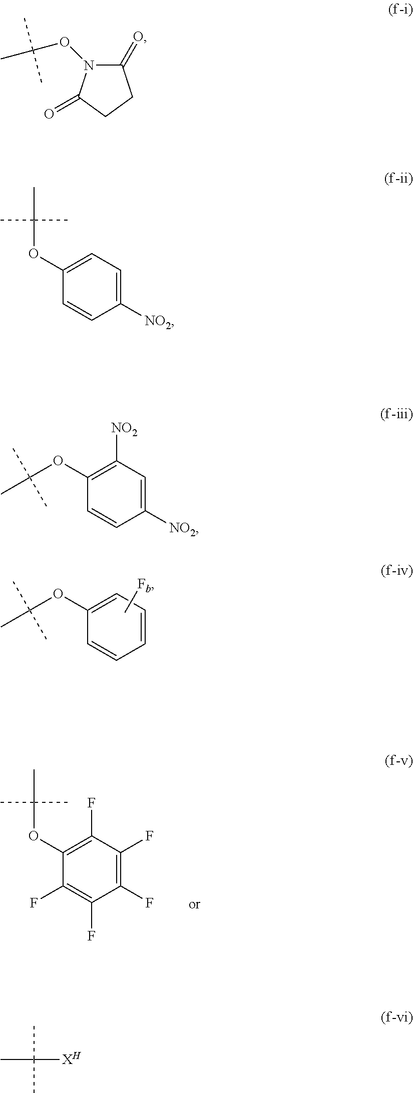

wherein
the dashed lines indicate attachment to the rest of the molecule,
b is 1, 2, 3 or 4
X^{H} is Cl, Br, I, or F.

It is understood that the moieties

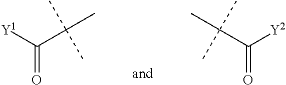

represent the at least two activated functional end groups.

Preferably, Y¹ and Y² of formula (VI) a structure of formula (f-i), (f-ii) or (f-v). More preferably, Y¹ and Y² of formula (VI) have a structure of formula (f-i) or (f-ii) and most preferably, Y¹ and Y² have a structure of formula (f-i).

Preferably, both moieties Y¹ and Y² of formula (VI) have the same structure. More preferably, both moieties Y¹ and Y² have the structure of formula (f-i).

Preferably, r1 of formula (VI) is 0.

Preferably, r1 and s1 of formula (VI) are both 0.

Preferably, one or more of the pair(s) R¹/R^{1a}, R²/R^{2a}, R³/R^{3a}, R⁴/R^{4a}, R¹/R², R³/R⁴, R^{1a}/R², and R^{3a}/R^{4a} of formula (VI) form a chemical bond or are joined together with the atom to which they are attached to form a C₃₋₈ cycloalkyl or form a ring A.

Preferably, one or more of the pair(s) R¹/R², R^{1a}/R^{2a}, R³/R⁴, R^{3a}/R^{4a} of formula (VI) are joined together with the atom to which they are attached to form a 4- to 7-membered heterocyclyl or 8- to 11-membered heterobicyclyl.

Preferably, the crosslinker reagent of formula (VI) is symmetric, i.e. the moiety

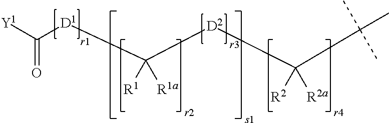

has the same structure as the moiety

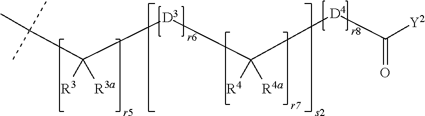

In one preferred embodiment s1, s2, r1 and r8 of formula (VI) are 0.

In another preferred embodiment s1, s2, r1 and r8 of formula (VI) are 0 and r4 of formula (VI) and r5 are 1.

Preferred crosslinker reagents are of formula (VI-1) to (VI-55):

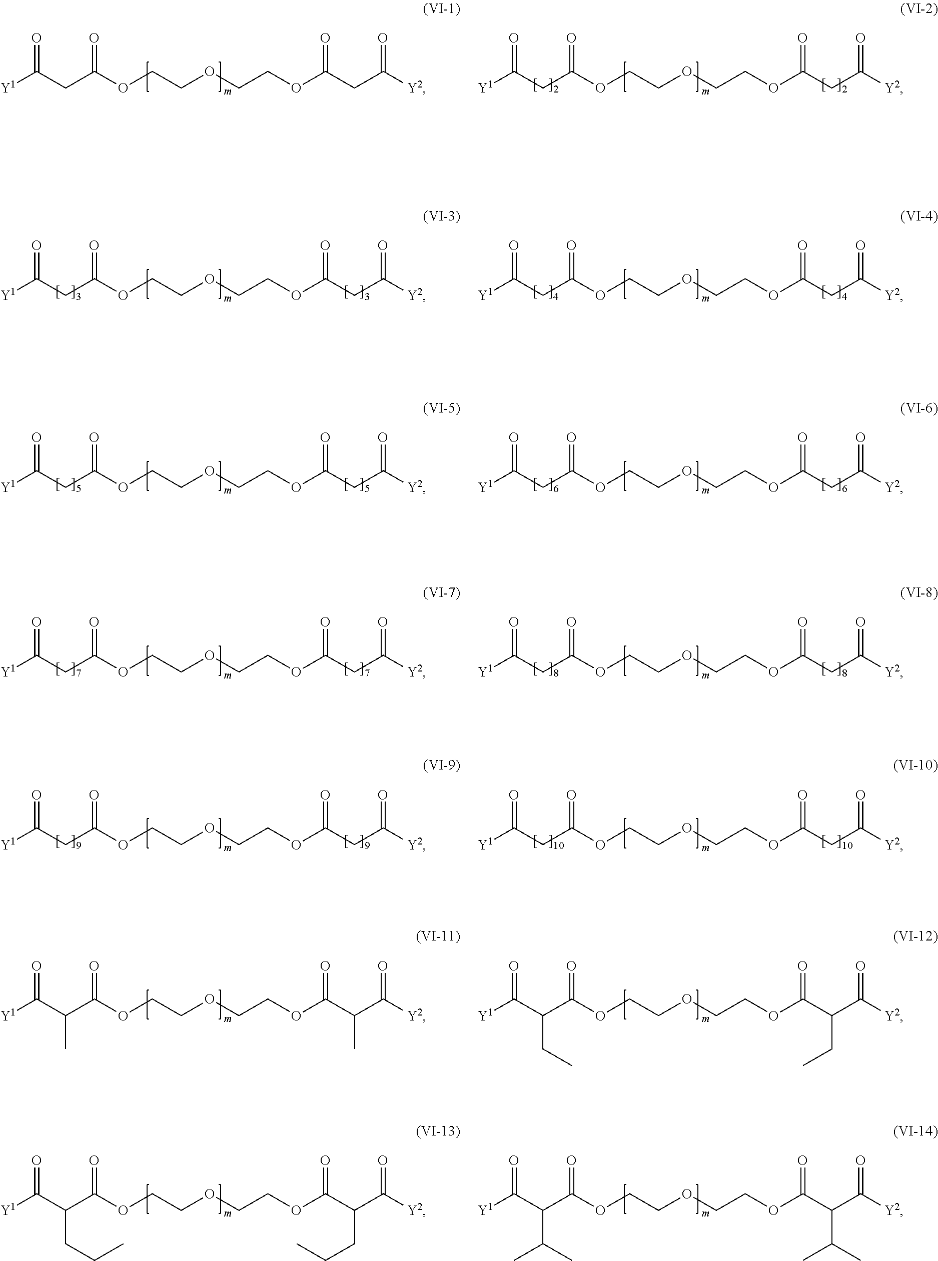

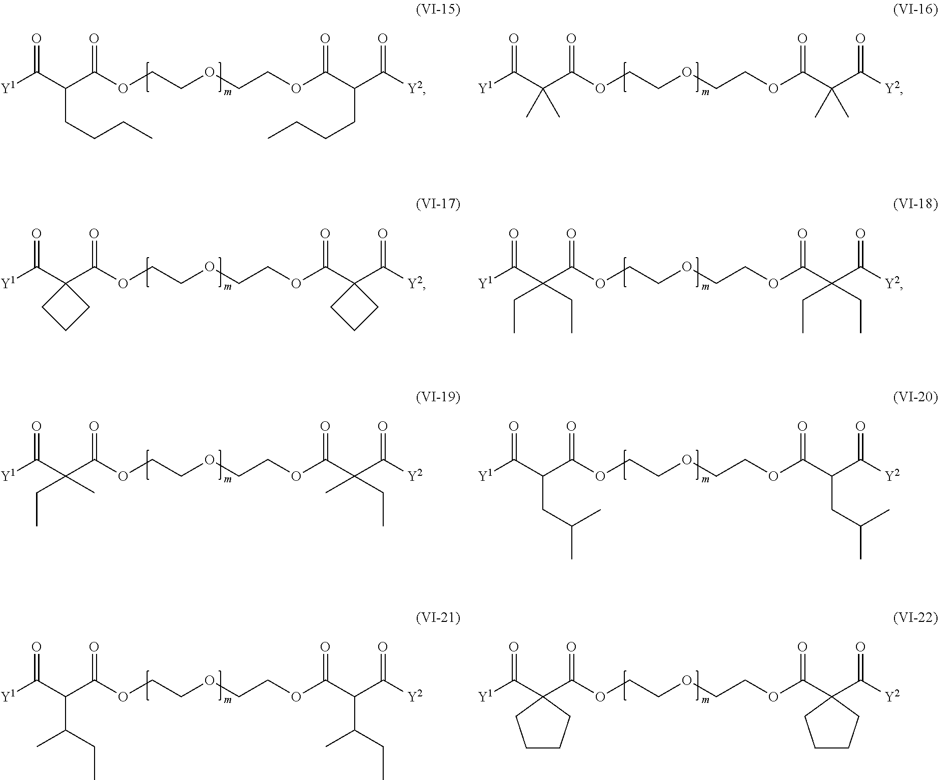

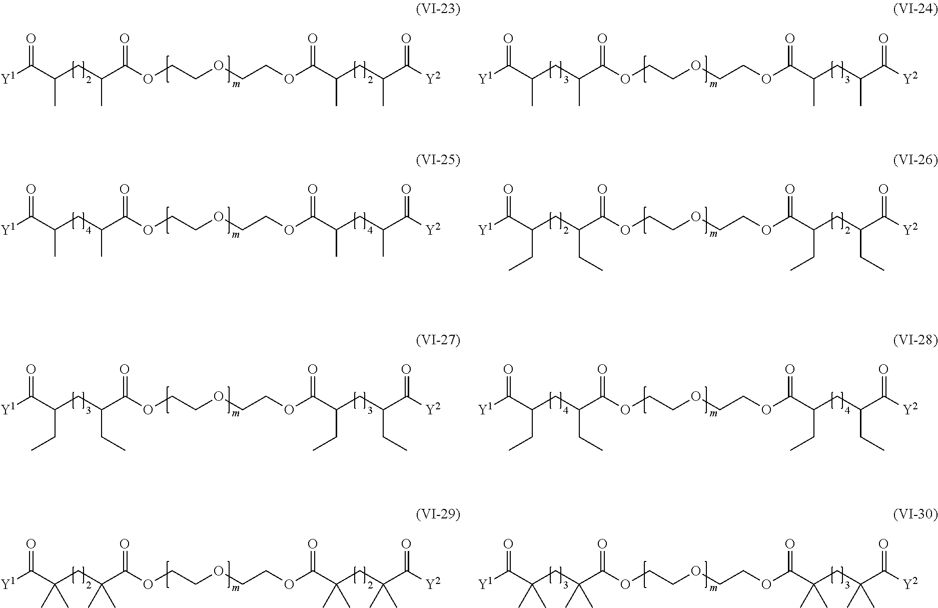

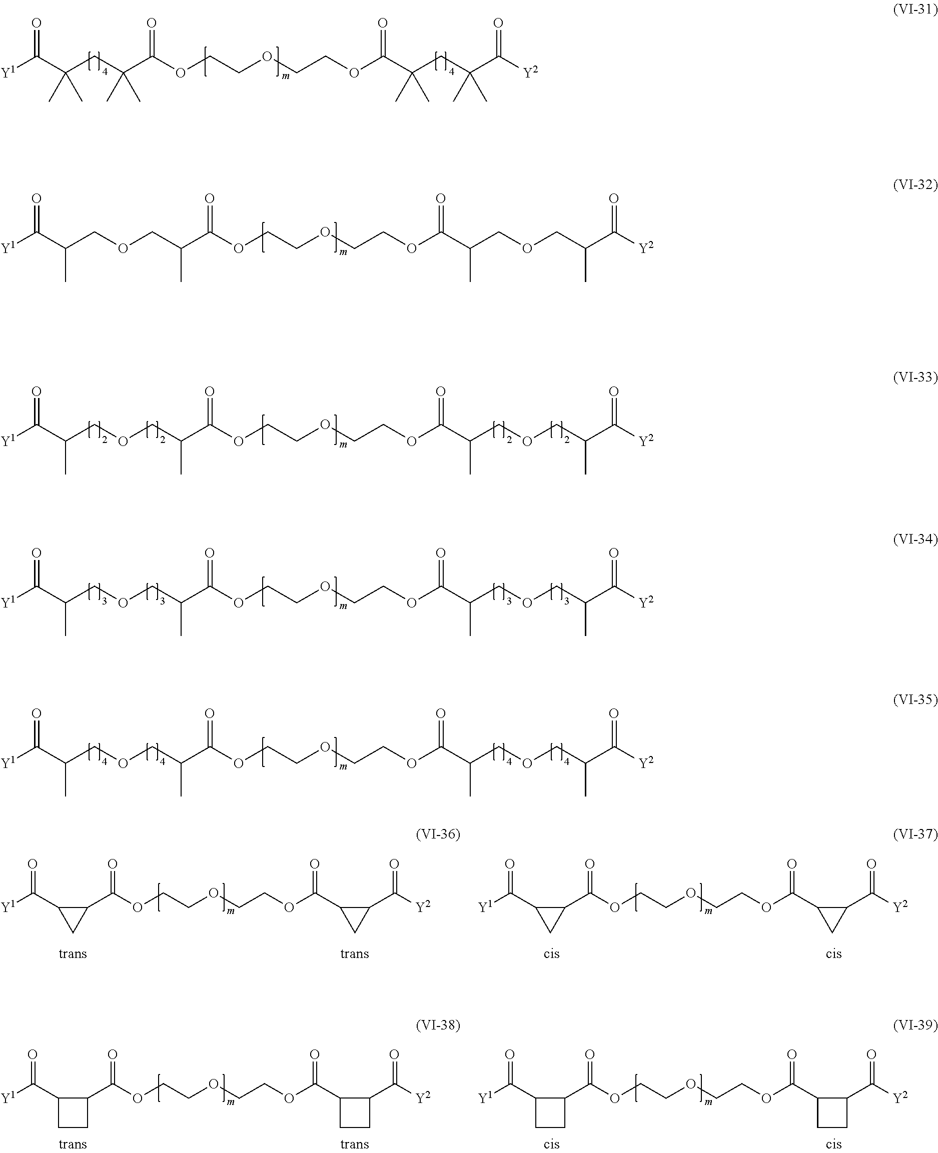

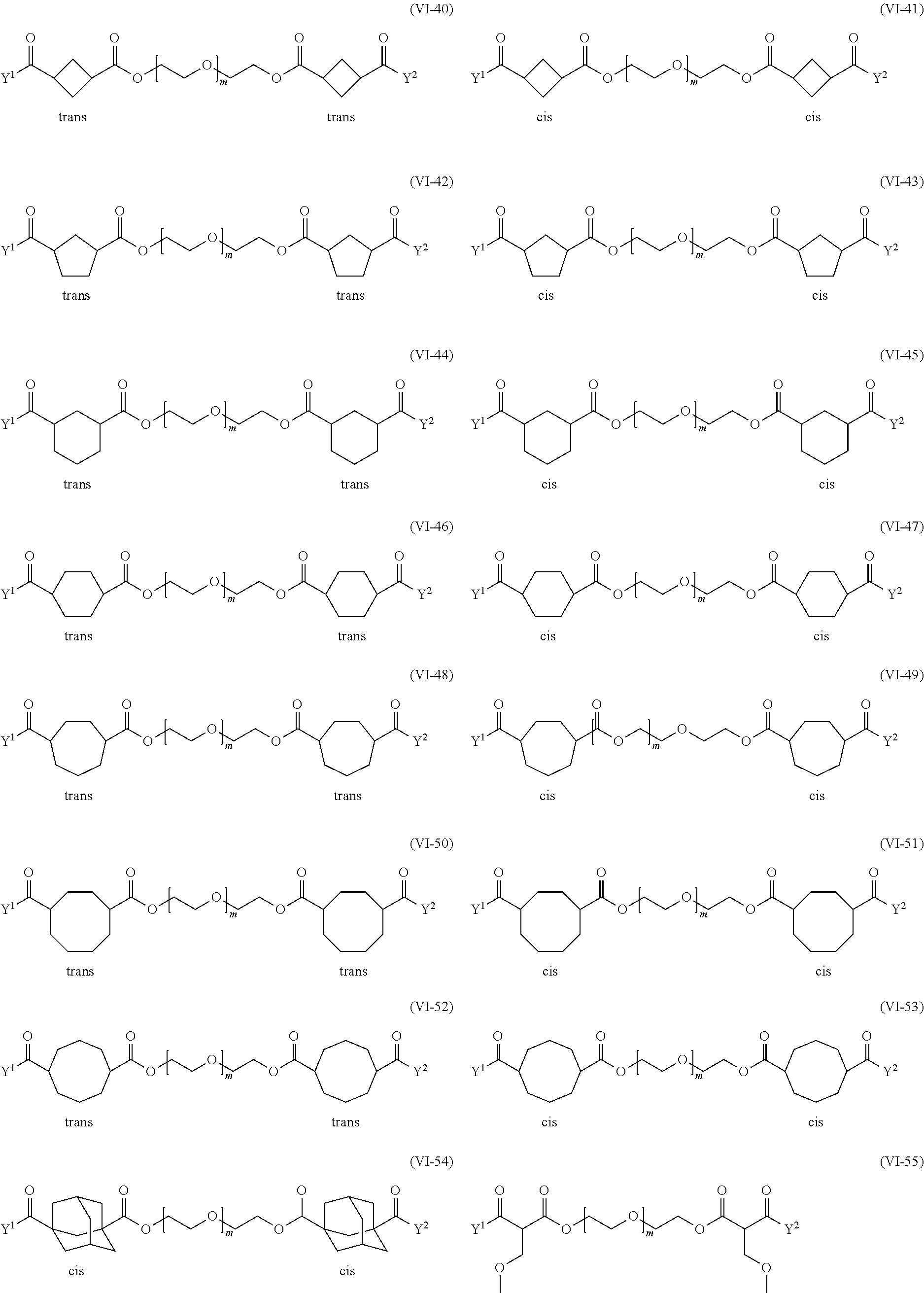

wherein
each crosslinker reagent may be in the form of its racemic mixture, where applicable; and
m, Y¹ and Y² are defined as above.

In one preferred embodiment, the crosslinker reagent is of VI-11 to VI-55, VI-1 and VI-2. Most preferred is crosslinker reagent VI-14.

In another embodiment, crosslinker reagents VI-1, VI-2, VI-5, VI-6, VI-7, VI-8, VI-9, VI-10, VI-11, VI-12, VI-13, VI-14, VI-15, VI-16, VI-17, VI-18, VI-19, VI-20, VI-21, VI-22, VI-23, VI-24, VI-25, VI-26, VI-27, VI-28, VI-29, VI-30, VI-31, VI-32, VI-33, VI-34, VI-35, VI-36, VI-37, VI-38, VI-39, VI-40, VI-41, VI-42, VI-43, VI-44, VI-45, VI-46, VI-47, VI-48, VI-49, VI-50, VI-51, VI-52, VI-53, VI-54 and VI-55 are preferred crosslinker reagents. More preferably, the at least one crosslinker reagent is of formula VI-5, VI-6, VI-7, VI-8, VI-9, VI-10, VI-14, VI-22, VI-23, VI-43, VI-44, VI-45 or VI-46, and most preferably, the at least one crosslinker reagent is of formula VI-5, VI-6, VI-9 or VI-14.

The preferred embodiments of the compound of formula (VI) as mentioned above apply accordingly to the preferred compounds of formulas (VI-1) to (VI-55).

The hydrogel contains from 0.01 to 2 mmol/g primary amine groups, more preferably from 0.02 to 1.8 mmol/g primary amine groups, even most preferably from 0.05 to 1.5 mmol/g primary amine groups. The term “X mmol/g primary amine groups” means that 1 g of dry hydrogel comprises X mmol primary amine groups. Measurement of the amine content of the hydrogel is carried out according to Gude et al. (Letters in Peptide Science, 2002, 9(4): 203-206, which is incorporated by reference in its entirety) and is also described in detail in the Examples section.

Preferably, the term “dry” as used herein means having a residual water content of a maximum of 10%, preferably less than 5% and more preferably less than 2% (determined according to Karl Fischer). The preferred method of drying is lyophilization.

Optionally, the process for the preparation of a hydrogel further comprises the step of:
(d) reacting the hydrogel from step (b) or (c) with a spacer reagent of formula (VII)

A^{x1}-S⁰-A^{x2} (VII),
wherein
S⁰ is selected from the group comprising C₁₋₅₀ alkyl, C₂₋₅₀ alkenyl and C₂₋₅₀ alkynyl, which fragment is optionally interrupted by one or more group(s) selected from —NH—, —N(C₁₋₄ alkyl)-, —O—, —S, —C(O)—, —C(O)NH, —C(O)N(C₁₋₄ alkyl)-, —O—C(O)—, —S(O)—, —S(O)₂—, 4- to 7-membered heterocyclyl, phenyl and naphthyl;
A^{x1} is a functional group for reaction with A^{x0}; and
A^{x2} is a functional group;
in the presence of a solvent to obtain a hydrogel-spacer conjugate.

Preferably, A^{x1} is selected from the group comprising activated carboxylic acid; Cl—(C═O)—; NHS—(C═O)—, wherein NHS is N-hydroxysuccinimide; ClSO₂—; R¹(C═O)—; I—; Br—; Cl—; SCN—; and CN—,
wherein
R¹ is selected from the group comprising H, C₁₋₆ alkyl, alkenyl, C₂₋₆ alkynyl, C₃₋₈ cycloalkyl, 4- to 7-membered heterocyclyl, 8- to 11-membered heterobicyclyl, phenyl, naphthyl, indenyl, indanyl, and tetralinyl.

Most preferably, A^{x1} is an activated carboxylic acid.

Suitable activating reagents to obtain the activated carboxylic acid are for example N,N′-dicyclohexyl-carbodiimide (DCC), 1-ethyl-3-carbodiimide (EDC), benzotriazol-1-yl-oxytripyrrolidinophosphonium hexafluorophosphate (PyBOP), bromotripyrrolidinophosphonium hexafluorophosphate (PyBrOP), 1-cyano-2-ethoxy-2-oxoethylidenaminooxy)dimethylamino-morpholino-carbenium hexafluorophosphate (COMU), 1-hydroxybenzotriazole (HOBT), 1-hydroxy-7-azabenzotriazole (HOAT), O-(6-chlorobenzotriazol-1-yl)-N,N,N′,N′-tetramethyluronium hexafluorophosphate (HCTU), 1-H-benzotriazolium (HBTU), (O-(7-azabenzotriazol-1-yl)-N,N,N′,N′-tetramethyluronium hexafluorophosphate (HATU), and O-(benzotriazol-1-yl)-N,N,N′,N′-tetramethyluronium tetrafluoroborate (TBTU). These reagents are commercially available and well-known to the skilled person.

Preferably, A^{x2} is selected from the group comprising—maleimide, —SH, —NH₂, —SeH, —N₃, —C≡CH, —CR¹═CR^{1a}R^{1b}, —OH, —(CH═X⁰)—R¹, —(C═O)—S—R¹, —(C═O)—H, —NH—NH₂, —O—NH₂, —Ar—X⁰, —Ar—Sn(R¹)(R^{1a})(R^{1b}), —Ar—B(OH)(OH),

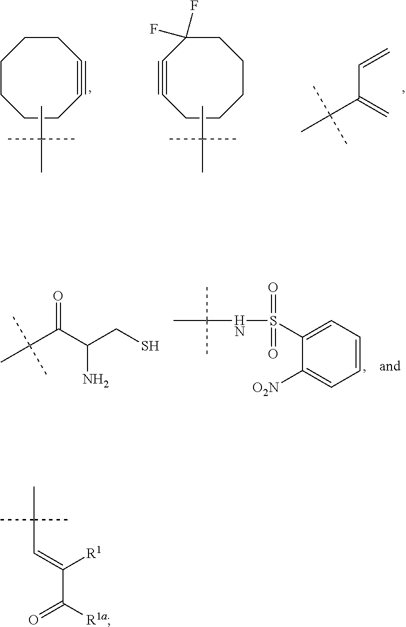

with optional protecting groups;
wherein
X⁰ is —OH, —NR¹R^{1a}, —SH, and —SeH,
Ar is selected from phenyl, naphthyl, indenyl, indanyl, and tetralinyl, and
R¹, R^{1a}, R^{1b} are independently of each other selected from the group comprising H, C₁₋₆ alkyl, C₂₋₆ alkenyl, C₂₋₆ alkynyl, C₃₋₈ cycloalkyl, 4- to 7-membered heterocyclyl, 8- to 11-membered heterobicyclyl, phenyl, naphthyl, indenyl, indanyl, and tetralinyl.

More preferably, A^{x2} is selected from —NH₂, maleimide and thiol and most preferably A^{x2} is maleimide. Equally preferred is thiol (—SH).

Suitable reaction conditions are described in the Examples sections and are known to the person skilled in the art.

Process step (d) may be carried out in the presence of a base. Suitable bases include customary inorganic or organic bases. These preferably include alkaline earth metal or alkali metal hydrides, hydroxides, amides, alkoxides, acetates, carbonates or bicarbonates such as, for example, sodium hydride, sodium amide, sodium methoxide, sodium ethoxide, potassium tert-butoxide, sodium hydroxide, potassium hydroxide, ammonium hydroxide, sodium acetate, potassium acetate, calcium acetate, ammonium acetate, sodium carbonate, potassium carbonate, potassium bicarbonate, sodium bicarbonate or ammonium carbonate, and tertiary amines such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN), N,N-diisopropylethylamine (DIPEA), diazabicycloundecene (DBU) or collidine.

Process step (d) may be carried out in the presence of a solvent. Suitable solvents for carrying out the process step (d) of the invention include organic solvents. These preferably include water and aliphatic, alicyclic or aromatic hydrocarbons such as, for example, petroleum ether, hexane, heptane, cyclohexane, methylcyclohexane, benzene, toluene, xylene or decalin; halogenated hydrocarbons such as, for example, chlorobenzene, dichlorobenzene, dichloromethane, chloroform, carbon tetrachloride, dichloroethane or trichloroethane; alcohols such as methanol, ethanol, n- or i-propanol, n-, i-, sec- or tert-butanol, ethanediol, propane-1,2-diol, ethoxyethanol, methoxyethanol, diethylene glycol monomethyl ether, dimethylether, diethylene glycol; acetonitrile, N-methyl-2-pyrrolidone (NMP), dimethylformamide (DMF), dimethyl sulfoxide (DMSO), N,N-dimethylacetamide, nitromethane, nitrobenzene, hexamethylphosphoramide (HMPT), 1,3-dimethyl-2-imidazolidinone (DMI), 1,3-dimethyl-3,4,5,6-tetrahydro-2(1H)-pyrimidinone (DMPU), ethyl acetate, acetone, butanone; ethers such as diethyl ether, diisopropyl ether, methyl t-butyl ether, methyl t-amyl ether, dioxane, tetrahydrofuran, 1,2-dimethoxyethane, 1,2-diethoxyethane or anisole; or mixtures thereof. Preferably, the solvent is selected from water, acetonitrile or N-methyl-2-pyrrolidone.

In one embodiment the hydrogel of the hydrogel-linked relaxin prodrug of the present invention is modified before a moiety L²-L¹-relaxin is conjugated to the hydrogel.

Preferably, the hydrogel is modified by a process comprising the steps of
(A) providing a hydrogel having groups A^{x0′}, wherein groups A^{x0′} represent the same or different, preferably same, functional groups;
(B) optionally covalently conjugating a spacer reagent of formula (VII)

A^{x1}-SP²-A^{x2} (VII),
wherein
SP² is C₁₋₅₀ alkyl, C₂₋₅₀ alkenyl or C₂₋₅₀ alkynyl, which C₁₋₅₀ alkyl, C₂₋₅₀ alkenyl and C₂₋₅₀ alkynyl is optionally interrupted by one or more group(s) selected from the group consisting of —NH—, —N(C₁₋₄ alkyl)-, —O—, —S, —C(O)—, —C(O)NH, —C(O)N(C₁₋₄ alkyl)-, —O—C(O)—, —S(O)—, —S(O)₂—, 4- to 7-membered heterocyclyl, phenyl and naphthyl;
A^{x1} is a functional group for reaction with A^{x0} of the hydrogel; and
A^{x2} is a functional group;
to A^{x0′} of the hydrogel from step (A); and
(C) reacting the hydrogel of step (A) or step (B) with a reagent of formula (VIII)

A^{x3}-Z⁰ (VIII),
wherein
A^{x3} is a functional group; and
Z⁰ is an inert moiety having a molecular weight ranging from 10 Da to 1000 kDa;
such that at most 99 mol-% of A^{x0} or A^{x2} react with A^{x3}.

Preferably, A^{x0′} of step (A) is selected from the group consisting of maleimide, amine (—NH₂ or —NH—), hydroxyl (—OH), carboxyl (—COOH) and activated carboxyl (—COY¹, wherein Y¹ is selected from formulas (f-i) to (f-vi):

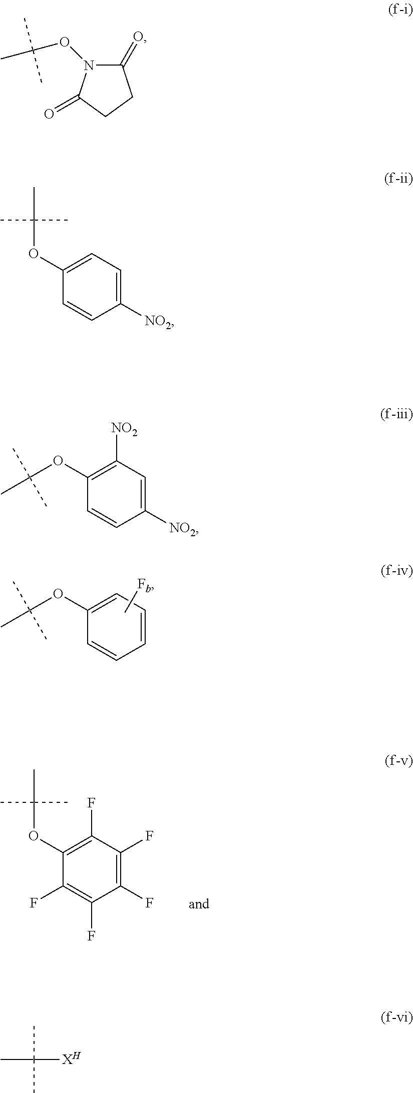

wherein
the dashed lines indicate attachment to the rest of the molecule,
b is 1, 2, 3 or 4;
X^{H} is Cl, Br, I, or F).

More preferably, A^{x0′} of step (A) is an amine or maleimide. Most preferably, A^{x0′} of step (A) is an amine.

It is understood that the functional groups A^{x0′} of step (A) correspond to A^{x0} of the at least one backbone reagent, if the hydrogel of the hydrogel-linked relaxin prodrug of the present invention is obtained from step (b) or (c) of the process described above, or to A^{x2}, if the hydrogel of the hydrogel-linked relaxin prodrug of the present invention is obtained from optional step (d).

In a preferred embodiment A^{x0′} of step (A) is an amine and A^{x1} of step (B) is ClSO₂—, R¹(C═O)—, I—, Br—, Cl—, SCN—, CN—, O═C═N—, Y¹—(C═O)—, Y¹—(C═O)—NH—, or Y¹—(C═O)—O—,
wherein
R¹ is H, C₁₋₆ alkyl, C₂₋₆ alkenyl, C₂₋₆ alkynyl, C₃₋₈ cycloalkyl, 4- to 7-membered heterocyclyl, 8- to 11-membered heterobicyclyl, phenyl, naphthyl, indenyl, indanyl, or tetralinyl; and
Y¹ is selected from formulas (f-i) to (f-vi):

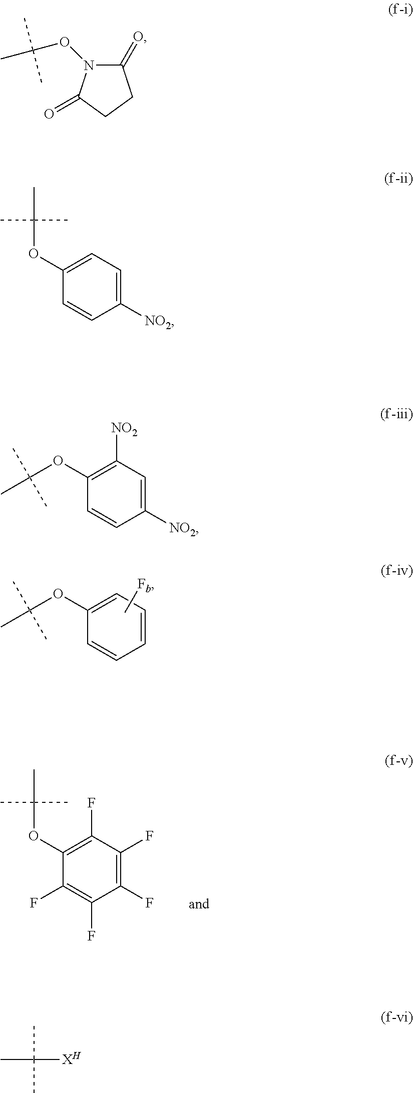

wherein
the dashed lines indicate attachment to the rest of the molecule,
b is 1, 2, 3 or 4,
X^{H} is Cl, Br, I, or F.

In another preferred embodiment A^{x0′} of step (A) is a hydroxyl group (—OH) and A^{x1} of step (B) is O═C═N—, I—, Br—, SCN—, or Y¹—(C═O)—NH—,
wherein Y¹ is selected from formulas (f-i) to (f-vi):

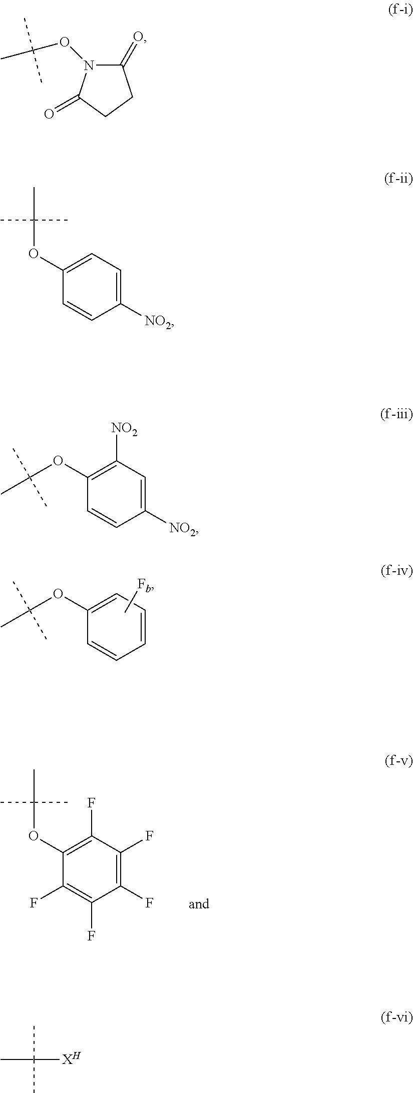

wherein
the dashed lines indicate attachment to the rest of the molecule,
b is 1, 2, 3 or 4,
X^{H} is Cl, Br, I, or F.

In another preferred embodiment A^{x0′} of step (A) is a carboxylic acid (—(C═O)OH) and A^{x1} of step (B) is a primary amine or secondary amine.

In another preferred embodiment A^{x0′} of step (A) is a maleimide and A^{x1} of step (B) is a thiol.

More preferably, A^{x0′} of step (A) is an amine and A^{x1} of step (B) is Y¹—(C═O)—, Y¹—(C═O)—NH—, or Y¹—(C═O)—O— and most preferably A^{x0′} of step (A) is an amine and A^{x1} of step (B) is Y¹—(C═O)—.

A^{x1} of step (B) may optionally be present in protected form.

Suitable activating reagents to obtain the activated carboxylic acid are for example N,N′-dicyclohexyl-carbodiimide (DCC), 1-ethyl-3-carbodiimide (EDC), benzotriazol-1-yl-oxytripyrrolidinophosphonium hexafluorophosphate (PyBOP), bromotripyrrolidinophosphonium hexafluorophosphate (PyBrOP), 1-cyano-2-ethoxy-2-oxoethylidenaminooxy)dimethylamino-morpholino-carbenium hexafluorophosphate (COMU), 1-hydroxybenzotriazole (HOBT), 1-hydroxy-7-azabenzotriazole (HOAT), O-(6-chlorobenzotriazol-1-yl)-N,N,N′,N′-tetramethyluronium hexafluorophosphate (HCTU), 1-H-benzotriazolium (HBTU), (O-(7-azabenzotriazol-1-yl)-N,N,N′,N′-tetramethyluronium hexafluorophosphate (HATU), and O-(benzotriazol-1-yl)-N,N,N′N′-tetramethyluronium tetrafluoroborate (TBTU). These reagents are commercially available and well-known to the skilled person.

Preferably, A^{x2} of step (B) is selected from the group consisting of -maleimide, —SH, —NH₂, —SeH, —N₃, —C≡CH, —CR¹═CR^{1a}R^{1b}, —OH, —(CH═X)—R¹, —(C═O)—S—R¹, —(C═O)—H, —NH—NH₂, —O—NH₂, —Ar—X⁰, —Ar—Sn(R¹)(R^{1a})(R^{1b}), —Ar—B(OH)(OH), Br, I, Y¹—(C═O)—, Y¹—(C═O)—NH—, Y¹—(C═O)—O—,

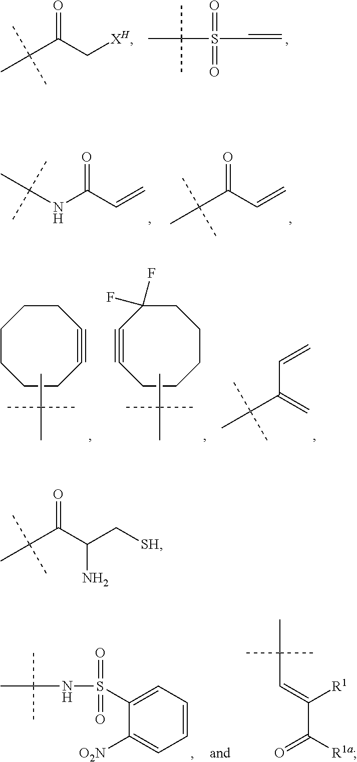

with optional protecting groups;
wherein
dashed lines indicate attachment to SP²;
X is O, S, or NH,
X⁰ is —OH, —NR¹R^{1a}, —SH, or —SeH,
X^{H} is Cl, Br, I or F;
Ar is phenyl, naphthyl, indenyl, indanyl, or tetralinyl;
R¹, R^{1a}, R^{1b} are independently of each other H, C₁₋₆ alkyl, C₂₋₆ alkenyl, C₂₋₆ alkynyl, C₃₋₈ cycloalkyl, 4- to 7-membered heterocyclyl, 8- to 11-membered heterobicyclyl, phenyl, naphthyl, indenyl, indanyl, or tetralinyl; and
Y¹ is selected from formulas (f-i) to (f-vi):

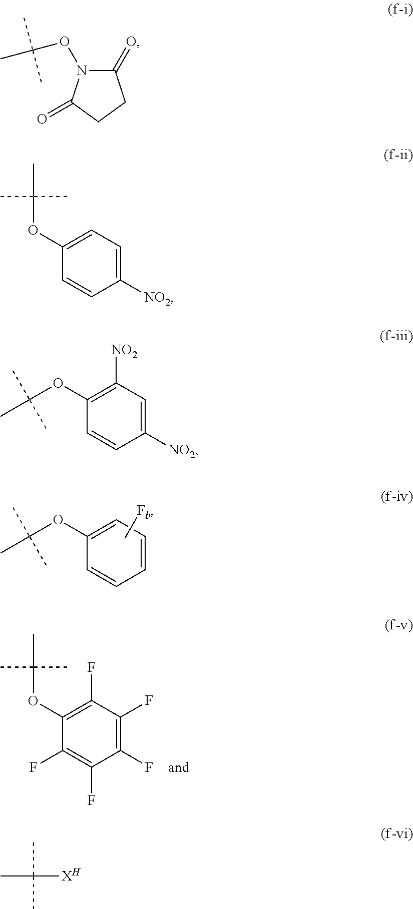

wherein
the dashed lines indicate attachment to the rest of the molecule,
b is 1, 2, 3 or 4,
X^{H} is Cl, Br, I, or F.

More preferably, A^{x2} of step (B) is —NH₂, maleimide or thiol and most preferably A^{x2} of step (B) is maleimide.

A^{x2} of step (B) may optionally be present in protected form.

If the hydrogel of step (A) is covalently conjugated to a spacer moiety, the resulting hydrogel-spacer moiety conjugate is of formula (IX):

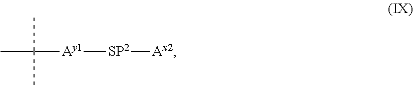

wherein
the dashed line indicates attachment to the hydrogel of step (A);
A^{y1} is the linkage formed between A^{x0′} and A^{x1}; and
SP² and A^{x2} are used as in formula (VII).

Preferably, A^{y1} of formula (IX) is a stable linkage.

Preferably, A^{y1} of formula (IX) is selected from the group consisting of

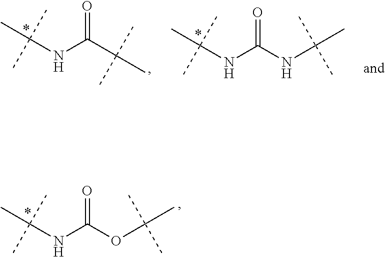

wherein
dashed lines marked with an asterisk indicate attachment to the hydrogel; and
unmarked dashed lines indicate attachment to SP².

Suitable reaction conditions are known to the person skilled in the art.

Process step (B) may be carried out in the presence of a base. Suitable bases include customary inorganic or organic bases. These preferably include alkaline earth metal or alkali metal hydrides, hydroxides, amides, alkoxides, acetates, carbonates or bicarbonates such as, for example, sodium hydride, sodium amide, sodium methoxide, sodium ethoxide, potassium tert-butoxide, sodium hydroxide, potassium hydroxide, ammonium hydroxide, sodium acetate, potassium acetate, calcium acetate, ammonium acetate, sodium carbonate, potassium carbonate, potassium bicarbonate, sodium bicarbonate or ammonium carbonate, and tertiary amines such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN), N,N-diisopropylethylamine (DIPEA), diazabicycloundecene (DBU) or collidine.

Process step (B) may be carried out in the presence of a solvent. Suitable solvents for carrying out the process step (B) of the invention include organic solvents. These preferably include water and aliphatic, alicyclic or aromatic hydrocarbons such as, for example, petroleum ether, hexane, heptane, cyclohexane, methylcyclohexane, benzene, toluene, xylene or decalin; halogenated hydrocarbons such as, for example, chlorobenzene, dichlorobenzene, dichloromethane, chloroform, carbon tetrachloride, dichloroethane or trichloroethane; alcohols such as methanol, ethanol, n- or i-propanol, n-, i-, sec- or tert-butanol, ethanediol, propane-1,2-diol, ethoxyethanol, methoxyethanol, diethylene glycol monomethyl ether, dimethylether, diethylene glycol; acetonitrile, N-methyl-2-pyrrolidone (NMP), dimethylformamide (DMF), dimethyl sulfoxide (DMSO), N,N-dimethylacetamide, nitromethane, nitrobenzene, hexamethylphosphoramide (HMPT), 1,3-dimethyl-2-imidazolidinone (DMI), 1,3-dimethyl-3,4,5,6-tetrahydro-2(1H)-pyrimidinone (DMPU), ethyl acetate, acetone, butanone; ethers such as diethyl ether, diisopropyl ether, methyl t-butyl ether, methyl t-amyl ether, dioxane, tetrahydrofuran, 1,2-dimethoxyethane, 1,2-diethoxyethane or anisole; or mixtures thereof. Preferably, the solvent is selected from the group consisting of water, acetonitrile and N-methyl-2-pyrrolidone.

Preferably, A^{x3} of step (C) is selected from the group consisting of —SH, —NH₂, —SeH, -maleimide, —C≡CH, —N₃, —CR¹═CR^{1a}R^{1b}, —(C═X)—R¹, —OH, —(C═O)—S—R¹, —NH—NH₂, —O—NH₂, —Ar—Sn(R¹)(R^{1a})(R^{1b}), —Ar—B(OH)(OH), —Ar—X⁰,

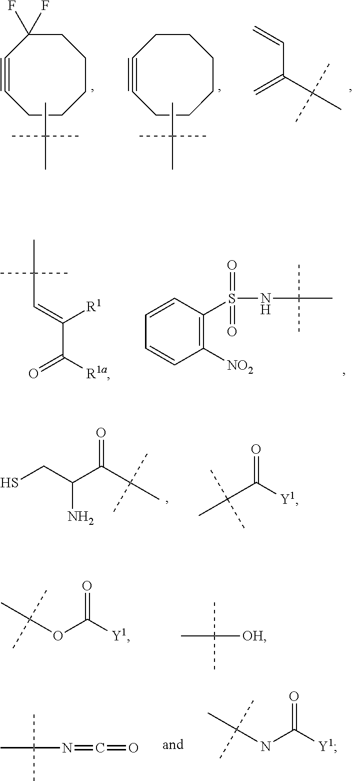

wherein
dashed lines indicate attachment to Z⁰;
X is O, S, or NH,
X⁰ is —OH, —NR¹R^{1a}, —SH, or —SeH;
R¹, R^{1a}, R^{1b} are independently of each other H, C₁₋₆ alkyl, C₂₋₆ alkenyl, C₂₋₆ alkynyl, C₃₋₈ cycloalkyl, 4- to 7-membered heterocyclyl, 8- to 11-membered heterobicyclyl, phenyl, naphthyl, indenyl, indanyl, or tetralinyl; and
Ar is phenyl, naphthyl, indenyl, indanyl, or tetralinyl.
Y¹ is an activated carboxylic acid, activated carbonate or activated carbamate, preferably Y¹ is selected from formulas (f-i) to (f-vi):

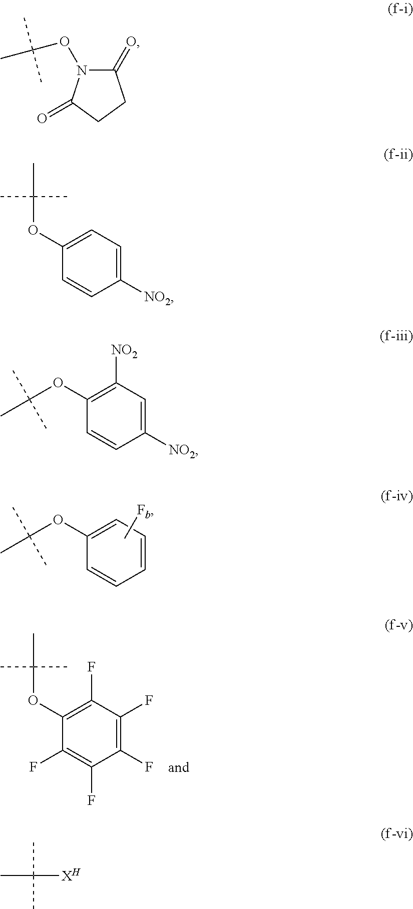

wherein
the dashed lines indicate attachment to the rest of the molecule,
b is 1, 2, 3 or 4,
X^{H} is Cl, Br, I, or F

In a preferred embodiment, Y¹ is selected from formulas (f-i) to (f-vi):

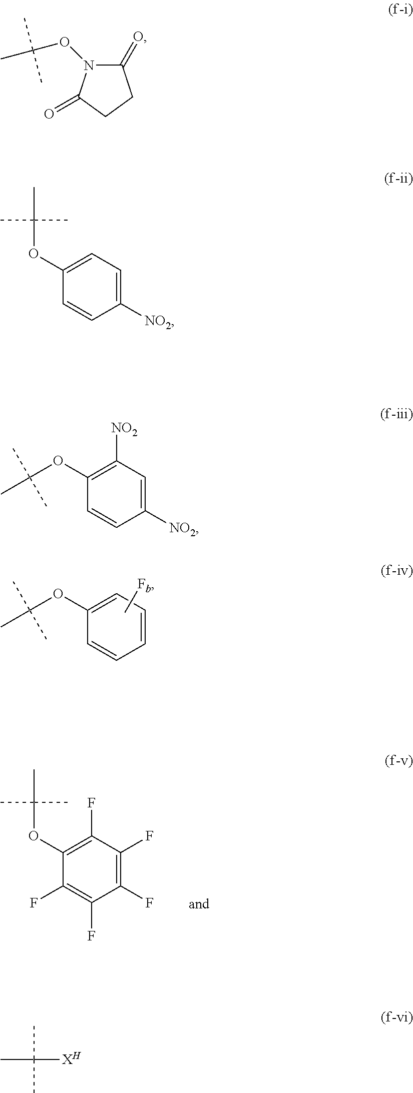

wherein
the dashed lines, b and X^{H} are used as above.

More preferably, A^{x3} of step (C) is —SH or -maleimide and most preferably A^{x3} of step (C) is —SH.

In another preferred embodiment A^{x3} is of formula (aI)

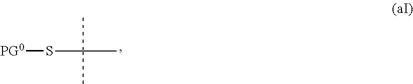

wherein
the dashed line indicates attachment to Z of formula (VIII);
PG⁰ is a sulfur-activating moiety; and
S is sulfur,

Preferably, PG⁰ of formula (aI) is selected from the group consisting of

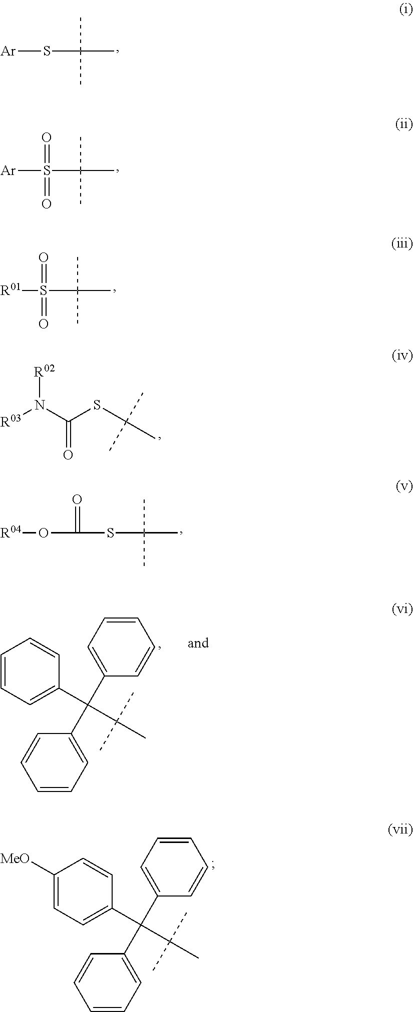

wherein
the dashed lines indicate attachment to the sulfur of formula (aI);
Ar is an aromatic moiety which is optionally further substituted;
R⁰¹, R⁰², R⁰³, R⁰⁴ are independently of each other —H; C₁₋₅₀ alkyl; C₂₋₅₀ alkenyl; or C₂₋₅₀ alkynyl, wherein C₁₋₅₀ alkyl; C₂₋₅₀ alkenyl; and C₂₋₅₀ alkynyl are optionally substituted with one or more R³, which are the same or different and wherein C₁₋₅₀ alkyl; C₂₋₅₀ alkenyl; and C₂₋₅₀ alkynyl are optionally interrupted by one or more groups selected from the group consisting of -Q-, —C(O)O—; —O—; —C(O)—; —C(O)N(R⁴)—; —S(O)₂N(R⁴)—; —S(O)N(R⁴)—; —S(O)₂—; —S(O)—; —N(R⁴)S(O)₂N(R^{4a})—; —S—; —N(R⁴)—; —OC(O)R⁴; —N(R⁴)C(O)—; —N(R⁴)S(O)₂—; —N(R⁴)S(O)—; —N(R⁴)C(O)O—; —N(R⁴)C(O)N(R^{4a})—; and —OC(O)N(R⁴R^{4a});
Q is selected from the group consisting of phenyl; naphthyl; indenyl; indanyl; tetralinyl; C₃₋₁₀ cycloalkyl; 4- to 7-membered heterocyclyl; and 8- to 11-membered heterobicyclyl, wherein T is optionally substituted with one or more R³, which are the same or different;
R³ is halogen; —CN; oxo (═O); —COOR⁵; —OR⁵; —C(O)R⁵; —C(O)N(R⁵R^{5a}); —S(O)₂N(R⁵R^{5a}); —S(O)N(R⁵R^{5a}); —S(O)₂R⁵; —S(O)R; —N(R⁵)S(O)₂N(R⁵R^{5b}); —SR⁵; —N(R⁵R^{5a}); —NO₂; —OC(O)R⁵; —N(R⁵)C(O)R^{5a}; —N(R⁵)S(O)₂R^{5a}; —N(R⁵)S(O)R^{5a}; —N(R⁵)C(O)OR^{5a}; —N(R⁵)C(O)N(R^{5a}R^{5b}); —OC(O)N(R⁵R^{5a}); or C₁₋₆ alkyl, wherein C₁₋₆ alkyl is optionally substituted with one or more halogen, which are the same or different; and
   R⁴, R^{4a}, R⁵, R^{5a}, R^{5b} are independently selected from the group consisting of —H; or C₁₋₆ alkyl, wherein C₁₋₆ alkyl is optionally substituted with one or more halogen, which are the same or different.

Preferably, R⁰¹, R⁰³ and R⁰⁴ are independently of each other C₁₋₆ alkyl.

Preferably, R⁰² is selected from H and C₁₋₆ alkyl.

Preferably, Ar is selected from the group consisting of

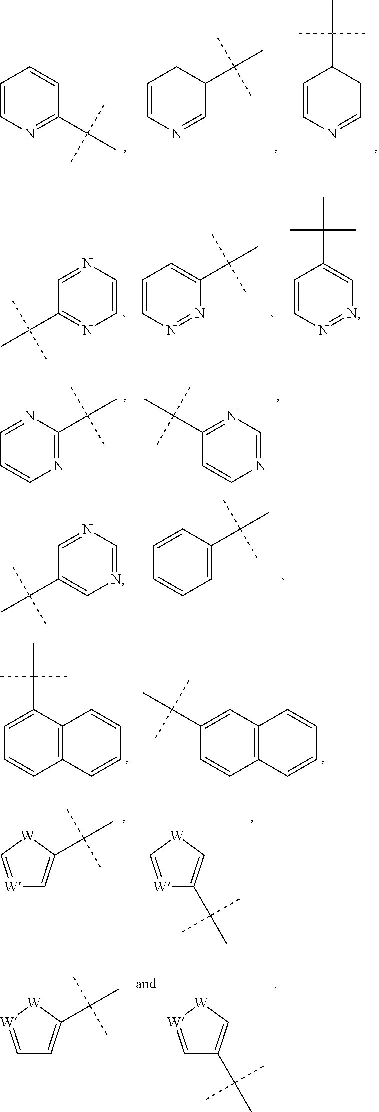

wherein
dashed lines indicate attachment to the rest of PG⁰ of formula (aI);
W is independently of each other O, S, or N;

### W′ is N; and

wherein Ar is optionally substituted with one or more substituent(s) independently selected from the group consisting of NO₂, Cl and F.

More preferably, PG⁰ of formula (aI) is selected from the group consisting of

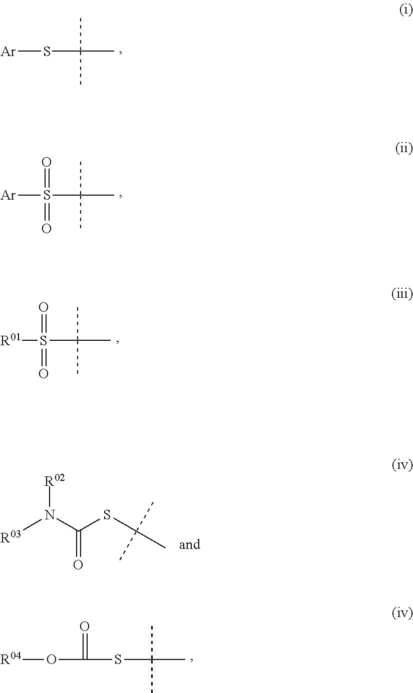

wherein
the dashed lines indicate attachment to the sulfur of formula (aI); and
Ar, R⁰¹, R⁰², R⁰³ and R⁰⁴ are used as above.

More preferably, PG⁰ of formula (aI) is

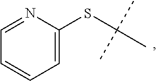

wherein
the dashed line indicates attachment to the sulfur of formula (aI).

A^{x3} of step (C) may optionally be present in protected form.

Preferred combinations of A^{x2} of step (B) and A^{x3} of step (C) are the following:

| A^{x2} | A^{x3} |
|---|---|
| -maleimide | HS—, H₂N—, or HSe— |
| —SH, —NH₂, or —SeH | maleimide- |
| —NH₂ | Y¹—(C═O)—, Y¹—(C═O)—NH—, |
| | or Y¹—(C═O)—O— |
| —N₃ | 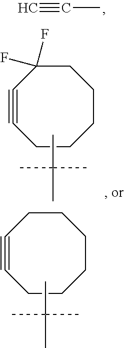 |
| 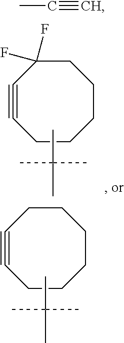 | N₃— |
| —CR^{1a}═CR^{1a}R^{1b} | 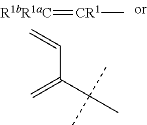 |
| 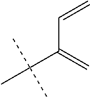 | R^{1b}R^{1a}C═CR¹— |
| —(C═X)—R¹ | 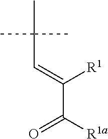 |
| 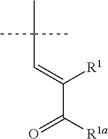 | R¹—(C═X)— |
| —OH | 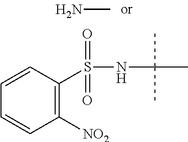 |
| 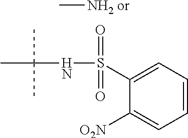 | HO— |
| —(C═O)—S—R¹ | 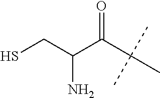 |
| 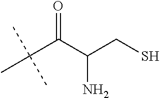 | R¹—S—(C═O)— |
| (C═O)—H | H₂N—NH—or H₂N—O— |
| —NH—NH₂ or —O—NH₂ | H—(C═O)— |
| —Ar—X⁰ | Ar—Sn(R¹)(R^{1a})(R^{1b}) or |
| | —Ar—B(OH)(OH) |
| (R^{1b})(R^{1a})(R¹)Sn—Ar—or | X0—Ar— |
| —Ar—B(OH)(OH) | |

wherein
X is O, S, or NH;
X⁰ is —OH, —NR¹R^{1a}, —SH, or —SeH;
R¹, R^{1a}, R^{1b} are independently of each other selected from the group consisting of H, C₁₋₆ alkyl, C₂₋₆ alkenyl, C₂₋₆ alkynyl, C₃₋₈ cycloalkyl, 4- to 7-membered heterocyclyl, 8- to 11-membered heterobicyclyl, phenyl, naphthyl, indenyl, indanyl, and tetralinyl; and
Ar is phenyl, naphthyl, indenyl, indanyl, or tetralinyl.

In another preferred embodiment A^{x2} is —SH and A^{x3} is of formula (aI), wherein PG⁰ is of formula (i), (ii), (iii), (iv), (v), (vi) or (viii). More preferably, PG⁰ of formula (aI) is of formula (i), (ii), (iii), (iv) or (v) and even more preferably, PG⁰ of formula (aI) is of formula (i). Most preferably, PG⁰ of formula (aI) is of formula

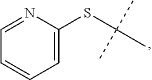

wherein
the dashed line indicates attachment to the sulfur of formula (aI).

In one preferred embodiment, A^{x2} of step (B) is an amine and A^{x3} of step (C) is Y¹—(C═O)—, Y¹—(C═O)—NH—, or Y¹—(C═O)—O— and most preferably A^{x2} of step (B) is an amine and A^{x3} of step (C) is Y¹—(C═O)—.

In another preferred embodiment A^{x2} of step (B) is maleimide and A^{x3} of step (C) is —SH.

In one embodiment the optional step (B) is omitted, A^{x0′} of step (A) is an amine and A^{x3} of step (C) is ClSO₂—, R¹(C═O)—, I—, Br—, Cl—, SCN—, CN—, O═C═N—, Y¹—(C═O)—, Y¹—(C═O)—NH—, or Y¹—(C═O)—O—,
wherein
R¹ is H, C₁₋₆ alkyl, C₂₋₆ alkenyl, C₂₋₆ alkynyl, C₃₋₈ cycloalkyl, 4- to 7-membered heterocyclyl, 8- to 11-membered heterobicyclyl, phenyl, naphthyl, indenyl, indanyl, or tetralinyl; and
Y¹ is selected from formulas (f-i) to (f-vi):

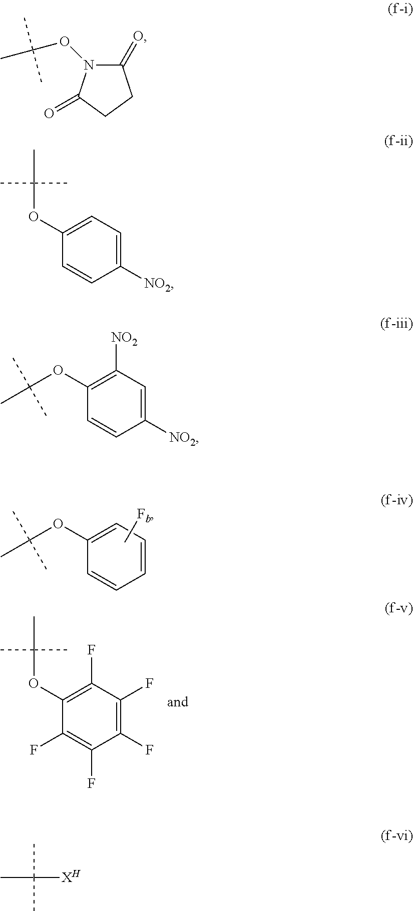

wherein
the dashed lines indicate attachment to the rest of the molecule,
b is 1, 2, 3 or 4,
X^{H} is Cl, Br, I, or F.

In another embodiment the optional step (B) is omitted, A^{x0′} of step (A) is a hydroxyl group (—OH) and A^{x3} of step (C) is O═C═N—, I—, Br—, SCN—, or Y¹—(C═O)—NH—,
wherein Y¹ is selected from formulas (f-i) to (f-vi):

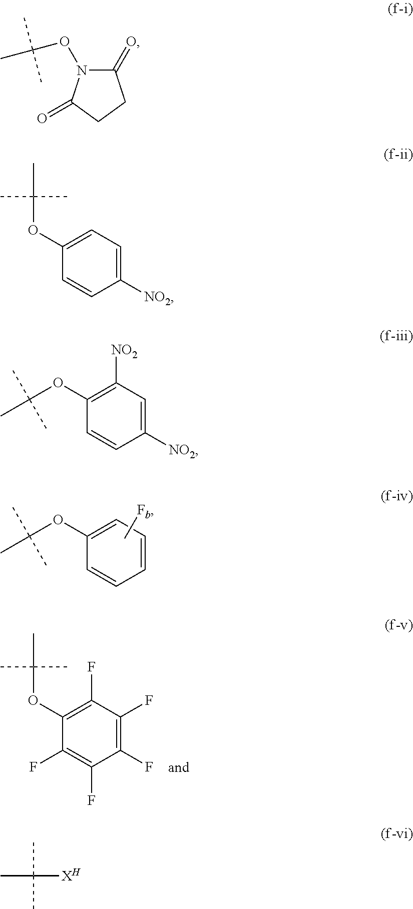

wherein
the dashed lines indicate attachment to the rest of the molecule,
b is 1, 2, 3 or 4,
X^{H} is Cl, Br, I, or F.

In another embodiment the optional step (B) is omitted, A^{x0′} of step (A) is a carboxylic acid (—(C═O)OH) and A^{x3} of step (C) is a primary amine or secondary amine.

In another embodiment the optional step (B) is omitted, A^{x0′} of step (A) is an amine and A^{x3} of step (C) is Y¹—(C═O)—, Y¹—(C═O)—NH—, or Y¹—(C═O)—O—.

In another embodiment the optional step (B) is omitted, A^{x0′} of step (A) is a maleimide and A^{x3} of step (C) is thiol.

In a preferred embodiment the optional step (B) is omitted, A^{x0′} of step (A) is an amine and A^{x3} of step (C) is Y¹—(C═O)—.

In another preferred embodiment the optional step (b) is omitted, A^{x0′} is —SH and A^{x3} is of formula (aI), wherein PG⁰ is of formula (i), (ii), (iii), (iv), (v), (vi) or (viii). More preferably, PG⁰ of formula (aI) is of formula (i), (ii), (iii), (iv) or (v) and even more preferably, PG⁰ of formula (aI) is of formula (i). Most preferably, PG⁰ of formula (aI) is of formula

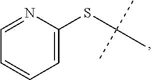

wherein
the dashed line indicates attachment to the sulfur of formula (aI).

The hydrogel obtained from step (C) has the structure of formula (Xa) or (Xb):

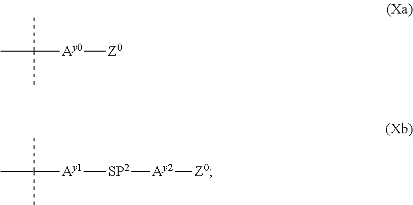

wherein
the dashed line indicates attachment to the hydrogel of step (A);
A^{y0} is the linkage formed between A^{x0′} and A^{x3};
A^{y1} is used as in formula (IX);
A^{y2} is the linkage formed between A^{x2} and A^{x3};
SP² is used as in formula (VII); and
Z⁰ is used as in formula (VIII).

Preferably, A^{y0} of step (A) and A^{y2} of formula (Xb) are selected from the group consisting of amide, carbamate,

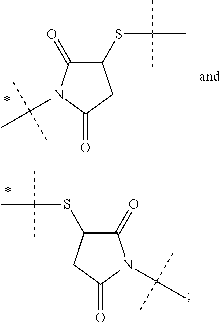

wherein
the dashed lines marked with an asterisk indicate attachment to the hydrogel or SP², respectively; and
the unmarked dashed lines indicate attachment to Z⁰ of formula (VIII).

In one embodiment, Z⁰ of step (C) is selected from the group consisting of C₁₋₅₀ alkyl, C₂₋₅₀ alkenyl, C₂₋₅₀ alkynyl, C₃₋₁₀ cycloalkyl, 4- to 7-membered heterocyclyl, 8- to 11-membered heterobicyclyl, phenyl; naphthyl; indenyl; indanyl; and tetralinyl; which C₁₋₅₀ alkyl, C₂₋₅₀ alkenyl, C₂₋₅₀ alkynyl, C₃₋₁₀ cycloalkyl, 4- to 7-membered heterocyclyl, 8- to 11-membered heterobicyclyl, phenyl; naphthyl; indenyl; indanyl; and tetralinyl are optionally substituted with one or more R¹⁰, which are the same or different and wherein C₁₋₅₀ alkyl; C₂₋₅₀ alkenyl; and C₂₋₅₀ alkynyl are optionally interrupted by one or more group(s) selected from the group consisting of T, —C(O)O—; —O—; —C(O)—; —C(O)N(R⁹)—; —S(O)₂N(R⁹)—; —S(O)N(R⁹)—; —S(O)₂—; —S(O)—; —N(R⁹)S(O)₂N(R^{9a})—; —S—; —N(R⁹)—; —OC(O)R⁹; —N(R⁹)C(O)—; —N(R⁹)S(O)₂—; —N(R⁹)S(O)—; —N(R⁹)C(O)O—; —N(R⁹)C(O)N(R^{9a})—; and —OC(O)N(R⁹R^{9a});
wherein
R⁹, R^{9a} are independently selected from the group consisting of H; T; C₁₋₅₀ alkyl; C₂₋₅₀ alkenyl; and C₂₋₅₀ alkynyl, which T; C₁₋₅₀ alkyl; C₂₋₅₀ alkenyl; and C₂₋₅₀ alkynyl are optionally substituted with one or more R¹⁰, which are the same or different and which C₁₋₅₀ alkyl; C₂₋₅₀ alkenyl; and C₂₋₅₀ alkynyl are optionally interrupted by one or more group(s) selected from the group consisting of T, —C(O)O—; —O—; —C(O)—; —C(O)N(R¹¹)—; —S(O)₂N(R¹¹)—; —S(O)N(R¹¹)—; —S(O)₂—; —S(O)—; —N(R¹¹)S(O)₂N(R^{11a})—; —S—; —N(R¹¹)—; —OC(O)R¹¹; —N(R¹¹)C(O)—; N(R¹¹)S(O)₂—; —N(R¹¹)S(O)—; —N(R¹¹)C(O)O—; —N(R¹¹)C(O)N(R^{11a})—; and —OC(O)N(R¹¹R^{11a});
T is selected from the group consisting of phenyl; naphthyl; indenyl; indanyl; tetralinyl; C₃₋₁₀ cycloalkyl; 4- to 7-membered heterocyclyl; and 8- to 11-membered heterobicyclyl, wherein T is optionally substituted with one or more R¹⁰, which are the same or different;
R¹⁰ is halogen; CN; oxo (═O); COOR¹²; OR¹²; C(O)R¹²; C(O)N(R¹²R^{12a}); S(O)₂N(R¹²R^{12a}); S(O)N(R¹²R^{12a}); S(O)₂R¹²; S(O)R¹²; N(R¹²)S(O)₂N(R^{12a}R^{12b}); SR¹²; N(R¹²R^{12a}); NO₂; OC(O)R¹²; N(R¹²)C(O)R^{12a}; N(R¹²)S(O)₂R^{12a}; N(R¹²)S(O)R^{12a}; N(R¹²)C(O)OR^{12a}; N(R¹²)C(O)N(R^{12a}R^{12b}); OC(O)N(R¹²R^{12a}); or C₁₋₆ alkyl, which C₁₋₆ alkyl is optionally substituted with one or more halogen, which are the same or different;
R¹¹, R^{11a}, R¹², R^{12a}, R^{12b} are independently of each other selected from the group consisting of H; and C₁₋₆ alkyl, which C₁₋₆ alkyl is optionally substituted with one or more halogen, which are the same or different.

In another embodiment Z⁰ of step (C) is an inert polymer having a molecular weight ranging from 0.5 kDa to 1000 kDa, preferably having a molecular weight ranging from 0.5 to 500 kDa, more preferably having a molecular weight ranging from 0.75 to 250 kDa, even more preferably ranging from 1 to 100 kDa, even more preferably ranging from 5 to 60 kDa, even more preferably from 10 to 50 and most preferably Z has a molecular weight of 40 kDa.

Preferably, Z⁰ of step (C) is an inert polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In a preferred embodiment Z⁰ of step (C) is an inert linear or branched PEG-based polymer comprising at least 70% PEG or a hyaluronic acid-based polymer comprising at least 70% hyaluronic acid. More preferably, Z⁰ of step (C) is an inert linear or branched PEG-based polymer comprising at least 70% PEG, even more preferably comprising at least 80% PEG and most preferably comprising at least 90% PEG.

In another preferred embodiment Z⁰ of step (C) is a zwitterionic polymer. Preferrably, such zwitterionic polymer comprises poly(amino acids) and/or poly(acrylates).

As used herein, the terms “zwitterion” and “zwitterionic” refer to a neutral molecule or moiety with positive and negative charges at different locations within that molecule or moiety at the same time.

According to Zhang et al. (Nature Biotechnology, 2013, volume 31, number 6, pages 553-557) hydrogels made of zwitterionic polymers resist the foreign body response.

Step (C) comprises reacting the hydrogel of step (A) or step (B) with a reagent of formula (VIII) in such manner that no more than 99 mol-% of A^{x0′} or A^{x2} react with A^{x3}. This can be achieved, for example, by reacting at most 0.99 chemical equivalents of the reagent of formula (VIII) relative to A^{x0′} or A^{x2} with the hydrogel of step (A) or (B).

In order to prevent the reaction of more than 0.99 chemical equivalents, the reagent of formula (VIII) can be used in an amount of at most 0.99 chemical equivalents relative to A^{x0′} or A² or, alternatively, the reaction rate is monitored and the reaction is interrupted when at most 0.99 chemical equivalents relative to A^{x0′} or A2 have reacted, especially when more than 0.99 chemical equivalents are used. It is understood that also due to physical constraints, such as steric hindrance, hydrophobic properties or other characteristics of the inert moiety Z, no more than 0.99 chemical equivalents may be capable of reacting with A^{x0′} or A^{x2}, even if more chemical equivalents are added to the reaction.

Preferably, step (C) comprises reacting the hydrogel of step (A) or step (B) with a reagent of formula (VIII) in such manner that no more than 80 mol-% of A^{x0′} or A^{x2} react with A^{x3}, even more preferably, such that no more than 60 mol-% of A^{x0′} or A^{x2} react with A^{x3}, even more preferably, such that no more than 40 mol-% of A^{x0′} or A^{x2} react with A^{x3}, even more preferably, such that no more than 20 mol-% of A^{x0′} or A^{x2} react with A^{x3} and most preferably, such that no more than 15 mol-% of A^{x} or A^{x2} react with A^{x3}.

This can be achieved, for example, by reacting at most 0.8, 0.6, 0.4, 0.2 or 0.15 chemical equivalents of the reagent of formula (VIII) relative to A^{x0′} or A^{x2} with the hydrogel of step (A) or (B), respectively.

Methods to prevent the reaction of more chemical equivalents are described above.

Based on the measurements of the amount of substance of A^{x0′} of step (A) and after step (C) the amount of substance of reacted A^{x0′} can be calculated with equation (1):

Amount of substance of reacted A^{x0′} in mmol/g=(A^{x0′}₁−A^{x0′}₂)/(A^{x0′}₂×MW_{Z}+1), (1)
wherein
A^{x0′}₁ is the amount of substance of functional groups A^{x0′} of the hydrogel of step (A) in mmol/g;
A^{x0′}₂ is the amount of substance of functional groups A^{x0′} of the hydrogel after step (C) in mmol/g; and
MW_{Z} is the molecular weight of Z in g/mmol.

If the optional spacer reagent was covalently conjugated to the hydrogel of step (A), the calculation of the number of reacted A^{x2} is done accordingly.

The percentage of reacted functional groups A^{x0′} relative to the functional groups A^{x0′} of the hydrogel of step (A) is calculated according to equation (2):

mol-% of reacted A^{x0′}=100×[(A^{x0′}₁−A^{x0′}₂)/(A^{x0′}₂×MW_{Z}+1)]/A^{x0′}₁, (2)
wherein the variables are used as above.

In one embodiment Z⁰ of step (C) is conjugated to the surface of the hydrogel. This can be achieved by selecting the size and structure of the reagent A^{x3}-Z⁰ such that it is too large to enter the pores or network of the hydrogel. Accordingly, the minimal size of A^{x3}-Z⁰ depends on the properties of the hydrogel. The person skilled in the art however knows methods how to test whether a reagent A^{x3}-Z⁰ is capable of entering into the hydrogel using standard experimentation, for example by using size exclusion chromatography with the hydrogel as stationary phase.

Suitable reversible prodrug linker moieties are for example disclosed in WO2005/099768 A2, WO2006/136586 A2, WO2009/095479 A2, WO2011/012722 A1, WO2011/089214 A1, WO2011/089216 A1, WO2011/089215 A1 and WO2013/160340 A1, which are herewith incorporated by reference.

In a preferred embodiment, the carrier-linked relaxin prodrug comprises, preferably is, a moiety D-L, wherein
(i) -D is a relaxin moiety;
   and
(ii) -L comprises, preferably is, a reversible linker moiety -L¹ represented by formula (I),

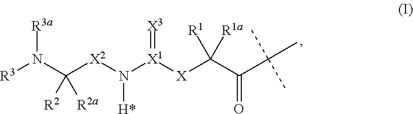

wherein the dashed line indicates the attachment to a nitrogen of D by forming an amide bond;
X is C(R⁴R^{4a}); N(R⁴); O; C(R⁴R^{4a})—C(R⁵R^{5a}); C(R⁵R^{5a})—C(R⁴R^{4a}); C(R⁴R^{4a})—N(R⁶); N(R⁶)—C(R⁴R^{4a}); C(R⁴R^{4a})—O; O—C(R⁴R^{4a}); or C(R⁷R^{7a});
X¹ is C; or S(O);
X² is C(R⁸R^{8a}); or C(R⁸R^{8a})—C(R⁹R^{9a});
X³ is O; S; or N—CN;
R¹, R^{1a}, R², R^{2a}, R⁴, R^{4a}, R⁵, R^{5a}, R⁶, R⁸, R^{8a}, R⁹, R^{9a} are independently selected from the group consisting of H; and C₁₋₆ alkyl;
R³, R^{3a} are independently selected from the group consisting of H; and C₁₋₆ alkyl, provided that in case one of R³, R^{3a} or both are other than H they are connected to N to which they are attached through an SP³-hybridized carbon atom;
R⁷ is N(R¹⁰R^{10a}); or NR¹⁰—(C═O)—R¹¹;
R^{7a}, R¹⁰, R^{10a}, R¹¹ are independently of each other H; or C₁₋₆ alkyl;
Optionally, one or more of the pairs R^{1a}/R^{4a}, R^{1a}/R^{5a}, R^{1a}/R^{7a}, R^{4a}/R^{5a}, R^{5a}/R^{9a} form a chemical bond;
Optionally, one or more of the pairs R¹/R^{1a}, R²/R^{2a}, R⁴/R^{4a}, R⁵/R^{5a}, R⁸/R^{8a}, R⁹/R^{9a} are joined together with the atom to which they are attached to form a C₃₋₇cycloalkyl; or 4- to 7-membered heterocyclyl;
Optionally, one or more of the pairs R¹/R⁴, R¹/R⁵, R¹/R⁶, R¹/R^{7a}, R⁴/R⁵, R⁴/R⁶, R⁸/R⁹, R²/R³ are joined together with the atoms to which they are attached to form a ring A;
Optionally, R³/R^{3a} are joined together with the nitrogen atom to which they are attached to form a 4- to 7-membered heterocycle;
A is selected from the group consisting of phenyl; naphthyl; indenyl; indanyl; tetralinyl; C₃₋₁₀ cycloalkyl; 4- to 7-membered heterocyclyl; and 9- to 11-membered heterobicyclyl; and
wherein L¹ is substituted with one to four moieties L²-Z and wherein L¹ is optionally further substituted, provided that the hydrogen marked with the asterisk in formula (I) is not replaced by L²-Z or a substituent;
   wherein
   L² is a single chemical bond or a spacer; and
   Z is a carrier.

In one embodiment L¹ of formula (I) is not further substituted.

It is understood that if R³/R^{3a} are joined together with the nitrogen atom to which they are attached to form a 4- to 7-membered heterocycle, only such 4- to 7-membered heterocycles may be formed in which the atoms directly attached to the nitrogen are SP³-hybridized carbon atoms. In other words, such 4- to 7-membered heterocycle formed by R³/R^{3a} together with the nitrogen atom to which they are attached has the following structure:

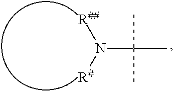

wherein
the dashed line indicates attachment to the rest of L¹;
the ring comprises 4 to 7 atoms comprising at least one nitrogen; and
R^{#} and R^{##} represent an SP³-hybridized carbon atom.

It is also understood that the 4- to 7-membered heterocycle may be further substituted.

Exemplary embodiments of suitable 4- to 7-membered heterocycles formed by R³/R^{3a} together with the nitrogen atom to which they are attached are the following:

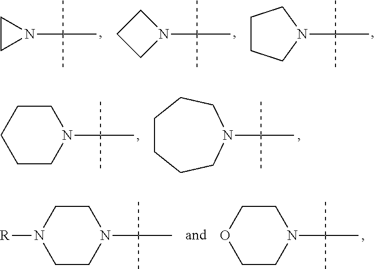

wherein
dashed lines indicate attachment to the rest of the molecule; and
R is selected from the group consisting of H and C₁₋₆ alkyl.

It is also understood that the 4- to 7-membered heterocycle may be further substituted.

It is understood that Z corresponds to the carrier as described above and that all embodiments of the carrier as described above also apply to Z.

Preferably, Z is a hydrogel, more preferably a PEG-based hydrogel, i.e. the carrier-linked relaxin prodrug comprising the reversible linker moiety of formula (I) is a hydrogel-linked relaxin prodrug. When Z is a hydrogel, L¹ is substituted with one moiety L²-Z.

If Z is a hydrogel, preferred embodiments for such hydrogel are as described above.

Thus, in a preferred embodiment the present invention relates to a hydrogel-linked relaxin prodrug comprising relaxin or a pharmaceutically acceptable salt thereof, wherein a relaxin moiety is connected through a reversible linker moiety L¹ and a moiety L² to a hydrogel Z. It is understood that multiple moieties L²-L¹-D are conjugated to a hydrogel Z.

The relaxin moiety is connected to L¹ through an amine functional group of relaxin. This may be the N-terminal amine function group or an amine functional group provided by a lysine side chain. If the relaxin moiety is RLN2, such RLN2 moiety may be connected to L¹ through the N-terminal amine group of the A- or B-chain or through an amine group provided by the lysine at position 9 or 17 of the A-chain (A9 or A17, respectively) or through the amine group provided by the lysine at position 9 of the B-chain (B9). If the relaxin moiety is RLN3, such RLN3 moiety may be connected to L¹ through the N-terminal amine group of the A- or B-chain or through an amine group provided by the lysine at position 12 or 17 of the A-chain (A12 or A17, respectively).

In one embodiment all relaxin moieties connected to a carrier moiety, preferably a hydrogel carrier, are connected to L¹ through the same amine functional group.

In one embodiment all relaxin moieties connected to a carrier moiety, preferably a hydrogel carrier, are connected to L¹ through different amine functional groups. In this embodiment it is preferred that the relaxin moieties are connected to L¹ through an amine functional group provided by a lysine of either the A- or B-chain of relaxin.

L¹ may be optionally further substituted. In general, any substituent may be used as far as the cleavage principle is not affected, i.e. the hydrogen marked with the asterisk in formula (I) is not replaced and the nitrogen of the moiety

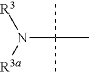

of formula (I) remains part of a primary, secondary or tertiary amine, i.e. R³ and R^{3a} are independently of each other H or are connected to N through an SP³-hybridized carbon atom.

Preferably, the one or more further optional substituent(s) of L¹ are independently selected from the group consisting of halogen; —CN; —COOR¹²; —OR¹²; —C(O)R¹²; —C(O)N(R¹²R^{12a}); —S(O)₂N(R¹²R^{12a}); —S(O)N(R¹²R^{12a}); —S(O)₂R¹²; —S(O)R¹²; —N(R¹²)S(O)₂N(R^{12a}R^{12b}); —SR¹²; —N(R¹²R^{12a}); —NO₂; —OC(O)R¹²; —N(R¹²)C(O)R^{12a}; —N(R¹²)S(O)₂R^{12a}; —N(R¹²)S(O)R^{12a}; —N(R¹²)C(O)OR^{12a}; —N(R¹²)C(O)N(R^{12a}R^{12b}); —OC(O)N(R¹²R^{12a}); Q; C₁₋₅₀ alkyl; C₂₋₅₀ alkenyl; and C₂₋₅₀ alkynyl, wherein Q; C₁₋₅₀ alkyl; C₂₋₅₀ alkenyl; and C₂₋₅₀ alkynyl are optionally substituted with one or more R³, which are the same or different and wherein C₁₋₅₀ alkyl; C₂₋₅₀ alkenyl; and C₂₋₅₀ alkynyl are optionally interrupted by one or more groups selected from the group consisting of Q, —C(O)O—; —O—; —C(O)—; —C(O)N(R¹⁴)—; —S(O)₂N(R¹⁴)—; —S(O)N(R¹⁴)—; —S(O)₂—; —S(O)—; —N(R¹⁴)S(O)₂N(R^{14a})—; —S—; —N(R¹⁴)—; —OC(O)R¹⁴; —N(R¹⁴)C(O)—; —N(R¹⁴)S(O)₂—; —N(R¹⁴)S(O)—; —N(R¹⁴)C(O)O—; —N(R¹⁴)C(O)N(R^{14a})—; and —OC(O)N(R¹⁴R^{14a})—;

R¹², R^{12a}, R^{12b} are independently selected from the group consisting of —H; Q; and C₁₋₅₀ alkyl; C₂₋₅₀ alkenyl; and C₂₋₅₀ alkynyl, wherein Q; C₁₋₅₀ alkyl; C₂₋₅₀ alkenyl; and C₂₋₅₀ alkynyl are optionally substituted with one or more R¹³, which are the same or different and wherein C₁₋₅₀ alkyl; C₂₋₅₀ alkenyl; and C₂₋₅₀ alkynyl are optionally interrupted by one or more groups selected from the group consisting of Q, —C(O)O—; —O—; —C(O)—; —C(O)N(R¹⁵)—; —S(O)₂N(R¹⁵)—; —S(O)N(R¹⁵)—; —S(O)₂—; —S(O)—; —N(R¹⁵)S(O)₂(R^{15a})—; —S—; —N(R¹⁵)—; —OC(O)R¹⁵; —N(R¹⁵)C(O)—; —N(R¹⁵)S(O)₂—; —N(R¹⁵)S(O)₂—; —N(R¹⁵)C(O)O—; —N(R¹⁵)C(O)N(R^{15a})—; and —OC(O)N(R¹⁵R^{15a});

Q is selected from the group consisting of phenyl; naphthyl; indenyl; indanyl; tetralinyl; C₃₋₁₀ cycloalkyl; 4- to 7-membered heterocyclyl; and 9- to 11-membered heterobicyclyl, wherein Q is optionally substituted with one or more R¹³, which are the same or different;

R¹³ is halogen; —CN; oxo (═O); —COOR¹⁶; —OR¹⁶; —C(O)R¹⁶; —C(O)N(R¹⁶R^{16a}); —S(O)₂N(R¹⁶R^{16a}); —S(O)N(R¹⁶R^{16a}); —S(O)₂R¹⁶; —S(O)R¹⁶; —N(R¹⁶)S(O)₂N(R¹⁶R^{16a}); —SR¹⁶; —N(R¹⁶R^{16a}); —NO₂; —OC(O)R¹⁶; —N(R¹⁶)C(O)R^{16a}; —N(R¹⁶)S(O)₂R¹⁶; —N(R¹⁶)S(O)R^{16a}; —N(R¹⁶)C(O)OR^{16a}; —N(R¹⁶)C(O)N(R^{16a}R^{16b}); —OC(O)N(R¹⁶R^{16a}); and C₁₋₆ alkyl, wherein C₁₋₆ alkyl is optionally substituted with one or more halogen, which are the same or different;

R¹⁴, R^{14a}, R¹⁵, R^{15a}, R¹⁶, R^{16a} and R^{16b} are independently selected from the group consisting of —H; and C₁₋₆ alkyl, wherein C₁₋₆ alkyl is optionally substituted with one or more halogen, which are the same or different.

More preferably, the one or more optional substituent(s) of L¹ are independently selected from the group consisting of halogen; —CN; —COOR¹²; —OR¹²; —C(O)R¹²; —C(O)N(R¹²R^{12a}); —S(O)₂N(R¹²R^{12a}); —S(O)N(R¹²R^{12a}); —S(O)₂R¹²; —S(O)R¹²; —N(R¹²)S(ON(R^{12a}R^{12b}); —SR¹²; —N(R¹²R^{12a}); —NO₂; —OC(O)R¹²; —N(R¹²)C(O)R^{12a}; —N(R¹²)S(O)₂R^{12a}; —N(R¹²)S(O)R^{12a}; —N(R¹²)C(O)OR^{12a}; —N(R¹²)C(O)N(R^{12a}R^{12b}); —OC(O)N(R¹²R^{12a}); Q; C₁₋₅₀ alkyl; C₂₋₅₀ alkenyl; and C₂₋₅₀ alkynyl, wherein Q; C₁₋₅₀ alkyl; C₂₋₅₀ alkenyl; and C₂₋₅₀ alkynyl are optionally substituted with one or more R¹³, which are the same or different and wherein C₁₋₅₀ alkyl; C₂₋₅₀ alkenyl; and C₂₋₅₀ alkynyl are optionally interrupted by one or more groups selected from the group consisting of Q, —C(O)O—; —O—; —C(O)—; —C(O)N(R¹⁴)—; —S(O)₂N(R¹⁴)—; —S(O)N(R¹⁴)—; —S(O)₂—; —S(O)—; —N(R¹⁴)S(O)₂N(R^{14a})—; —S—; —N(R¹⁴)—; —OC(O)R¹⁴; —N(R¹⁴)C(O)—; —N(R¹⁴)S(O)₂—; —N(R¹⁴)S(O)—; —N(R¹⁴)C(O)O—; —N(R¹⁴)C(O)N(R¹⁴)—; and —OC(O)N(R¹⁴R^{14a});

R¹², R^{12a}, R^{12b} are independently selected from the group consisting of H; Q; C₁₋₅₀ alkyl; C₂₋₅₀ alkenyl; and C₂₋₅₀ alkynyl, wherein Q; C₁₋₅₀ alkyl; C₂₋₅₀ alkenyl; and C₂₋₅₀ alkynyl are optionally substituted with one or more R¹⁰, which are the same or different and wherein C₁₋₅₀ alkyl; C₂₋₅₀ alkenyl; and C₂₋₅₀ alkynyl are optionally interrupted by one or more groups selected from the group consisting of Q, —C(O)O—; —O—; —C(O)—; —C(O)N(R¹⁵)—; —S(O)₂N(R¹⁵)—; —S(O)N(R¹⁵)—; —S(O)₂—; —S(O)—; —N(R¹⁵)S(O)₂N(R^{15a})—; —S—; —N(R¹⁵)—; —OC(O)R¹⁵; —N(R¹⁵)C(O)—; —N(R¹⁵)S(O)₂—; —N(R¹⁵)S(O)—; —N(R¹⁵)C(O)O—; —N(R¹⁵)C(O)N(R^{15a})—; and —OC(O)N(R¹⁵R^{15a});

Q is selected from the group consisting of phenyl; naphthyl; indenyl; indanyl; tetralinyl; C₃₋₁₀ cycloalkyl; 4- to 7-membered heterocyclyl; or 9- to 11-membered heterobicyclyl;

R¹³, R¹⁴, R^{14a}, R¹⁵ and R^{15a} are independently selected from H, halogen; and C₁₋₆ alkyl.

Even more preferably, the one or more optional substituent(s) of L¹ are independently selected from the group consisting of halogen; C₁₋₅₀ alkyl; C₂₋₅₀ alkenyl; and C₂₋₅₀ alkynyl, wherein C₁₋₅₀ alkyl; C₂₋₅₀ alkenyl; and C₂₋₅₀ alkynyl are optionally substituted with one or more R¹³;

R¹³ is selected from the group consisting of halogen, C₁₋₆ alkyl, C₂₋₆ alkenyl and C₂₋₆ alkynyl.

Most preferably, the one or more optional substituent(s) of L¹ are independently selected from the group consisting of halogen; C₁₋₆ alkyl; C₂₋₆ alkenyl; and C₂₋₆ alkynyl.

Preferably, a maximum of 6—H atoms of L¹ are independently replaced by a substituent, e.g. 5—H atoms are independently replaced by a substituent, 4—H atoms are independently replaced by a substituent, 3—H atoms are independently replaced by a substituent, 2—H atoms are independently replaced by a substituent, or 1—H atom is replaced by a substituent.

In general, L² can be attached to L¹ at any position apart from the replacement of the hydrogen marked with an asterisk in formula (I) and as long as R³ and R^{3a} are independently of each other H or are connected to N through an SP³-hybridized carbon atom.

Preferably, L²-Z is attached to R¹, R^{1a}, R², R^{2a}, R³, R^{3a}, R⁴, R^{4a}, R⁵, R^{5a}, R⁶, R^{7a}, R⁸, R^{8a}, R⁹ or R^{9a} of formula (I).

The term L²-Z is attached to R^{x″}, wherein R^{x} is R¹, R^{1a}, R², R^{2a}, R³, R^{3a}, R⁴, R^{4a}, R⁵, R^{5a}, R⁶, R^{7a}, R⁸, R^{8a}, R⁹ or R^{9a}, means that if R^{x} is H, this hydrogen is replaced by L²-Z; if R^{x} is C₁₋₆ alkyl then one of the hydrogen atoms of the C₁₋₆ alkyl is replaced by L²-Z; if R^{x} is H or C₁₋₆ alkyl and which H or C₁₋₆ alkyl are further substituted, then any hydrogen atom either of H directly or as provided by the C₁₋₆ alkyl or by the substituent may be replaced by L²-Z.

Preferably, L²-Z is attached to R³, R^{3a}, R⁴, R^{4a}, R⁵, R^{5a}, R⁶, R¹⁰, R^{10a} or R¹¹ of formula (I).

Even more preferably, L²-Z is attached to R³, R^{3a}, R¹⁰, R^{10a} or R¹¹ of formula (I) Z.

Even more preferably, L²-Z is attached to R¹⁰, R^{10a} or R¹¹ of formula (I).

Most preferably, L²-Z is attached to R¹¹ of formula (I).

Preferably, X of formula (I) is C(R⁷R^{7a}).

Preferably, R⁷ of formula (I) is NR¹⁰—(C═O)—R¹¹.

Preferably, R^{7a} of formula (I) is H.

Preferably, R¹⁰ of formula (I) is H, methyl, ethyl or propyl. More preferably, R¹⁰ of formula (I) is methyl.

Preferably, R¹¹ is H, methyl, ethyl or propyl. More preferably, R¹¹ of formula (I) is H.

Preferably, X¹ of formula (I) is C.

Preferably, X² of formula (I) is C(R⁸R^{8a}).

Preferably, R⁸ of formula (I) is H, methyl, ethyl or propyl. More preferably, R⁸ of formula (I) is H.

Preferably, R^{8a} of formula (I) is H, methyl, ethyl or propyl. More preferably, R^{8a} of formula (I) is H.

Preferably, R⁸ and R^{8a} of formula (I) are H.

Preferably, X³ of formula (I) is O.

Preferably, R¹ of formula (I) is H, methyl, ethyl or propyl. More preferably, R¹ of formula (I) is H.

Preferably, R^{1a} of formula (I) is H, methyl, ethyl or propyl. More preferably, R^{1a} of formula (I) is H.

Preferably, R¹ and R^{1a} of formula (I) are both H.

Preferably, R² of formula (I) is H, methyl, ethyl or propyl. More preferably, R² of formula (I) is H.

Preferably, R^{2a} of formula (I) is H, methyl, ethyl or propyl. More preferably, R^{2a} of formula (I) is H.

Preferably, R² and R^{2a} of formula (I) are H.

Preferably, R³ of formula (I) is H or methyl, ethyl or propyl. More preferably, R³ of formula (I) is H.

Preferably, R^{3a} of formula (I) is H or methyl, ethyl or propyl. More preferably, R^{3a} of formula (I) is methyl.

Preferably, R³ of formula (I) is H and R^{3a} of formula (I) is methyl.

In a preferred embodiment L¹ is of formula (II)

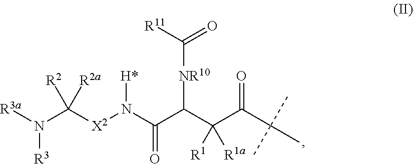

wherein
the dashed line indicates the attachment to a nitrogen of D by forming an amide bond; R¹, R^{1a}, R², R^{2a}, R³, R^{3a}, R¹⁰, R¹¹ and X² are used as defined in formula (I);
and wherein L¹ is optionally further substituted, provided that the hydrogel marked with the asterisk in formula (II) is not replaced by a substituent.

In one embodiment L¹ of formula (II) is not further substituted.

Even more preferably, L²-Z is attached to R³, R^{3a}, R¹⁰ or R¹¹ of formula (II).

Even more preferably, L²-Z is attached to R¹⁰ or R¹¹ of formula (II).

Most preferably, L²-Z is attached to R¹¹ of formula (II).

Preferably, X² of formula (II) is C(R⁸R^{8a}).

Preferably, R⁸ of formula (II) is H, methyl, ethyl or propyl. More preferably, R⁸ of formula (II) is H.

Preferably, R^{8a} of formula (II) is H, methyl, ethyl or propyl. More preferably, R^{8a} of formula (II) is H.

Preferably, R¹ of formula (II) is H, methyl, ethyl or propyl. More preferably, R¹ of formula (II) is H.

Preferably, R^{1a} of formula (II) is H, methyl, ethyl or propyl. More preferably, R^{1a} of formula (II) is H.

Preferably, R¹ and R^{1a} of formula (II) are H.

Preferably, R² of formula (II) is H, methyl, ethyl or propyl. More preferably, R² of formula (II) is H.

Preferably, R^{2a} of formula (II) is H, methyl, ethyl or propyl. More preferably, R^{2a} of formula (II) is H.

Preferably, R² and R^{2a} of formula (II) are H.

Preferably, R³ of formula (II) is H or methyl, ethyl or propyl. More preferably, R³ of formula (II) is H.

Preferably, R^{3a} of formula (II) is H or methyl, ethyl or propyl. More preferably, R^{3a} of formula (II) is methyl.

Preferably, R³ of formula (II) is H and R^{3a} of formula (II) is methyl.

Preferably, R¹⁰ of formula (II) is H, methyl, ethyl or propyl. More preferably, R¹⁰ of formula (II) is methyl.

Preferably, R¹¹ of formula (II) is H.

Even more preferably, L¹ is of formula (III):

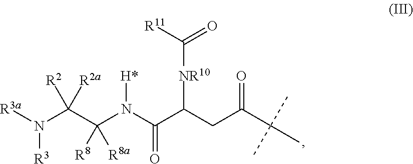

wherein
the dashed line indicates the attachment to a nitrogen of D by forming an amide bond; R², R^{2a}, R³, R^{3a}, R⁸, R^{8a}, R⁹, R^{9a}, R¹⁰, and R¹¹ are used as defined in formula (I);
and wherein L¹ is optionally further substituted, provided that the hydrogel marked with the asterisk in formula (III) is not replaced by a substituent.

In one embodiment L¹ of formula (III) is not further substituted.

Even more preferably, L²-Z is attached to R³, R^{3a}, R¹⁰ or R¹¹ of formula (III).

Even more preferably, L²-Z is attached to R¹⁰ or R¹¹ of formula (III).

Most preferably, L²-Z is attached to R¹¹ of formula (III).

Preferably, R² of formula (III) is H, methyl, ethyl or propyl. More preferably, R² of formula (III) is H.

Preferably, R^{2a} of formula (III) is H, methyl, ethyl or propyl. More preferably, R^{2a} of formula (III) is H.

Preferably, R² and R^{2a} of formula (II) are H.

Preferably, R³ of formula (III) is H or methyl, ethyl or propyl. More preferably, R³ of formula (III) is H.

Preferably, R^{3a} of formula (III) is H or methyl, ethyl or propyl. More preferably, R^{3a} of formula (III) is methyl.

Preferably, R³ of formula (III) is H and R^{3a} of formula (III) is methyl.

Preferably, R⁸ of formula (III) is H or methyl, ethyl or propyl. More preferably, R⁸ of formula (III) is H.

Preferably, R^{8a} of formula (III) is H or methyl, ethyl or propyl. More preferably, R^{8a} of formula (III) is methyl.

Preferably, R⁸ and R^{8a} of formula (III) are H.

Preferably, R¹⁰ of formula (III) is H, methyl, ethyl or propyl. More preferably, R¹⁰ of formula (III) is methyl.

Preferably, R¹¹ of formula (III) is H.

Even more preferably, L¹ is of formula (IV):

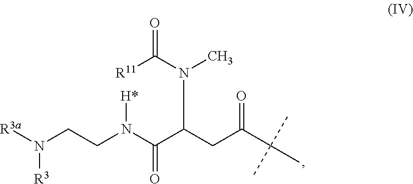

wherein
the dashed line indicates the attachment to a nitrogen of D by forming an amide bond;
R³ and R^{3a} are used as defined in formula (I);
R^{10b} is C₁₋₆ alkyl;
and wherein L¹ is optionally further substituted, provided that the hydrogel marked with the asterisk in formula (IV) is not replaced by a substituent.

In one embodiment L¹ of formula (IV) is not further substituted.

Even more preferably, L²-Z is attached to R³, R^{3a} or R¹¹ of formula (IV).

Most preferably, L²-Z is attached to R¹¹ of formula (IV).

Preferably, R³ of formula (IV) is H or methyl, ethyl or propyl. More preferably, R³ of formula (IV) is H.

Preferably, R^{3a} of formula (IV) is H or methyl, ethyl or propyl. More preferably, R^{3a} of formula (IV) is methyl.

Preferably, R³ of formula (IV) is H and R^{3a} of formula (IV) is methyl.

Preferably, R¹¹ of formula (IV) is H.

L² is a single chemical bond or a spacer.

When L² is other than a single chemical bond, L²-Z is preferably —C(O)N(R¹⁷)—; —S(O)₂N(R¹⁷)—; —S(O)N(R¹⁷)—; —N(R¹⁷)S(O)₂N(R^{17a})—; —N(R¹⁷)—; —OC(O)R¹⁷; —N(R¹⁷)C(O)—; —N(R¹⁷)S(O)₂—; —N(R¹⁷)S(O)—; —N(R¹⁷)C(O)O—; —N(R¹⁷)C(O)N(R^{17a})—; and —OC(O)N(R¹⁷R^{17a})—; Q; C₁₋₅₀ alkyl; C₂₋₅₀ alkenyl; or C₂₋₅₀ alkynyl, wherein Q; C₁₋₅₀ alkyl; C₂₋₅₀ alkenyl; and C₂₋₅₀ alkynyl are optionally substituted with one or more R¹⁸, which are the same or different and wherein C₁₋₅₀ alkyl; C₂₋₅₀ alkenyl; and C₂₋₅₀ alkynyl are optionally interrupted by one or more groups selected from the group consisting of Q, —C(O)O—; —O—; —C(O)—; —C(O)N(R¹⁹)—; —S(O)₂N(R¹⁹)—; —S(O)N(R¹⁹)—; —S(O)₂—; —S(O)—; —N(R¹⁹)S(O)₂N(R^{19a})—; —S—; —N(R¹⁹)—; —OC(O)R¹⁹; —N(R¹⁹)C(O)—; —N(R¹⁹)S(O)₂—; —N(R¹⁹)S(O)—; —N(R¹⁹)C(O)O—; —N(R¹⁹)C(O)N(R^{19a})—; and —OC(O)N(R¹⁹R^{19a});

R¹⁷, R^{17a}, R^{17b} are independently selected from the group consisting of —H; Z; Q; and C₁₋₅₀ alkyl; C₂₋₅₀ alkenyl; or C₂₋₅₀ alkynyl, wherein Q; C₁₋₅₀ alkyl; C₂₋₅₀ alkenyl; and C₂₋₅₀ alkynyl are optionally substituted with one or more R¹⁷, which are the same or different and wherein C₁₋₅₀ alkyl; C₂₋₅₀ alkenyl; and C₂₋₅₀ alkynyl are optionally interrupted by one or more groups selected from the group consisting of Q, —C(O)O—; —O—; —C(O)—; —C(O)N(R²⁰)—; —S(O)₂N(R²⁰)—; —S(O)N(R²⁰)—; —S(O)₂—; —S(O)—; —N(R²⁰)S(O)₂N(R^{20a})—; —S—; —N(R²⁰)—; —OC(O)R²⁰; —N(R²⁰)C(O)—; —N(R²⁰)S(O)₂—; —N(R²⁰)S(O)—; —N(R²⁰)C(O)O—; —N(R²⁰)C(O)N(R^{2a})—; and —OC(O)N(R²⁰R^{20a});

Q is selected from the group consisting of phenyl; naphthyl; indenyl; indanyl; tetralinyl; C₃₋₁₀ cycloalkyl; 4 to 7 membered heterocyclyl; or 9 to 11 membered heterobicyclyl, wherein Q is optionally substituted with one or more R¹⁷, which are the same or different;

R¹⁸ is Z; halogen; —CN; oxo (═O); —COOR²¹; —OR²¹; —C(O)R²¹; —C(O)N(R²¹R^{21a}); —S(O)₂N(R²¹R^{21a}); —S(O)N(R²¹R^{21a}); —S(O)₂R²¹; —S(O)R²¹; —N(R²¹)S(O)₂N(R^{21a}R^{21b}); —SR²¹; —N(R²¹R^{21a}); —NO₂; —OC(O)R²¹; —N(R²¹)C(O)R^{21a}; —N(R²¹)S(O)₂R^{21a}; —N(R²¹)S(O)R^{21a}; —N(R²¹)C(O)OR^{21a}; —N(R²¹)C(O)N(R^{21a}R^{21b}); —OC(O)N(R²¹R^{21a}); or C₁₋₆ alkyl, wherein C₁₋₆ alkyl is optionally substituted with one or more halogen, which are the same or different;

R¹⁹, R^{19a}, R²⁰, R^{20a}, R²¹, R^{21a} and R^{21b} are independently selected from the group consisting of —H; Z; or C₁₋₆ alkyl, wherein C₁₋₆ alkyl is optionally substituted with one or more halogen, which are the same or different;

provided that one of R¹⁷, R^{17a}, R^{17b}, R¹⁸, R¹⁹, R^{19a}, R²⁰, R^{20a}, R²¹, R^{21a} or R^{21b} is Z.

More preferably, L² is a C₁₋₂₀ alkyl chain, which is optionally interrupted by one or more groups independently selected from —O—; and —C(O)N(R^{1aa})—; and which C₁₋₂₀ alkyl chain is optionally substituted with one or more groups independently selected from OH; and —C(O)N(R^{1aa}R^{1aa}); wherein R^{1aa}, R^{1aaa} are independently selected from the group consisting of H; and C₁₋₄ alkyl.

Preferably, L² has a molecular weight in the range of from 14 g/mol to 750 g/mol.

Preferably, L² is attached to Z via a terminal group selected from

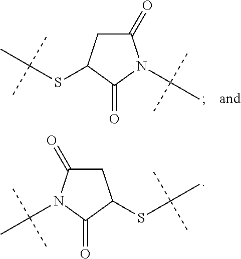

In case L² has such terminal group it is furthermore preferred that L² has a molecular weight in the range of from 14 g/mol to 500 g/mol calculated without such terminal group.

Preferably, L² is of formula (Ia)

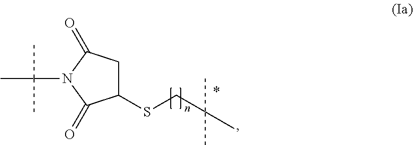

wherein
the dashed line marked with the asterisk indicates attachment to L¹ and the unmarked dashed line indicates attachment to Z; and
n is 1, 2, 3, 4, 5, 6, 7, 8, 9, 10, 11, 12, 13, 14 or 15.

Preferably, n of formula (Ia) is 1, 2, 3, 4, 5, 6, 7, or 8. More preferably, n of formula (Ia) is 2, 3, 4, 5, 6 or 7. Even more preferably, n of formula (Ia) is 3, 4, 5, 6 or 7. Even more preferably, n of formula (Ia) is 4, 5 or 6 and most preferably, n of formula (Ia) is 5.

Preferably, L is represented by formula (V):

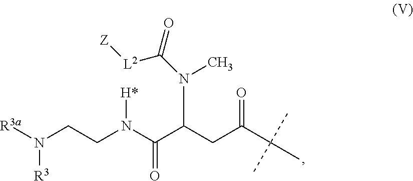

wherein
the dashed line indicates the attachment to a nitrogen of D by forming an amide bond;
R³, R^{3a}, L² and Z are used as defined in formula (I); and
R^{10b} is used as defined in formula (IV).

Preferably, R³ of formula (V) is H or methyl, ethyl or propyl. More preferably, R³ of formula (V) is H.

Preferably, R^{3a} of formula (V) is H or methyl, ethyl or propyl. More preferably, R^{3a} of formula (V) is methyl.

Preferably, R³ of formula (V) is H and R^{3a} of formula (V) is methyl.

Preferably, L² of formula (V) is of formula (Ia):

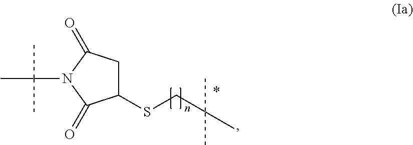

wherein
the dashed line marked with the asterisk indicates attachment to L¹ and the unmarked dashed line indicates attachment to Z; and
n is 1, 2, 3, 4, 5, 6, 7, 8, 9, 10, 11, 12, 13, 14 or 15.

Preferably, n of formula (Ia) is 1, 2, 3, 4, 5, 6, 7, or 8. More preferably, n of formula (Ia) is 2, 3, 4, 5, 6 or 7. Even more preferably, n of formula (Ia) is 3, 4, 5, 6 or 7. Even more preferably, n of formula (Ia) is 4, 5 or 6 and most preferably, n of formula (Ia) is 5.

In another embodiment, the carrier-linked relaxin prodrug comprises, preferably is, a moiety D-L, wherein
(i) -D is a relaxin moiety;
   and
(ii) -L comprises, preferably is, a reversible linker moiety -L¹ represented by formula (X):

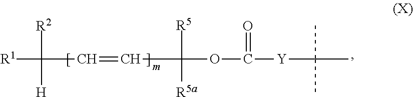

wherein
the dashed line indicates attachment to a hydroxyl group, thiol group or amine group of D, preferably to an amine group of D;
m is 0 or 1;
Y is NH or NR⁶CH₂, preferably NR⁶CH₂;
R¹ and R² are independently of each other CN; NO₂; C₆₋₁₈ aryl; C₆₋₁₈ heteroaryl; C₂₋₂₀ alkenyl, preferably C₂₋₆ alkenyl; C₂₋₂₀ alkynyl, preferably C₂₋₆ alkynyl; COR³; SOR³; SO₂R³; SR⁴; or one and only one of R¹ and R² may be H; C₁₋₂₀ alkyl, preferably C₁₋₆ alkyl; arylalkyl or heteroarylalkyl;
optionally, R¹ and R² may from a C₃₋₁₀ cycloalkyl, 4- to 7-membered heterocyclyl or 8- to 11-membered heterobicyclyl; preferably a C₃₋₁₀ cycloalkyl;
R³ is H; C₁₋₂₀ alkyl, preferably C₁₋₆ alkyl; C₆₋₁₈ aryl; C₆₋₁₈ heteroaryl; heteroarylalkyl; OR⁹ or NR⁹₂;
R⁴ is C₁₋₂₀ alkyl, preferably C₁₋₆ alkyl; C₆₋₁₈ aryl; arylalkyl; C₆₋₁₈ heteroaryl; or heteroarylalkyl;
R⁵, R^{5a} are independently of each other H; C₁₋₂₀ alkyl, preferably C₁₋₆ alkyl; C₂₋₂₀ alkenyl, preferably C₂₋₆ alkenyl; C₂₋₂₀ alkynly, preferably C₂₋₆ alkynyl; C₆₋₁₈ aryl; optionally substitute arylalkyl; C₆₋₁₈ heteroaryl; or optionally substituted heteroarylalkyl;
R⁶ is C₁₋₂₀ alkyl, preferably C₁₋₆ alkyl; C₆₋₁₈ aryl; optionally substituted arylalkyl; C₆₋₁₈ heteroaryl; or optionally substituted heteroarylalkyl;
each R⁹ is independently of each other H; or C₁₋₂₀ alkyl, preferably C₁₋₆ alkyl; or both R⁹ of a moiety NR⁹₂ form together with the nitrogen to which they are attached a 4- to 7-membered heterocyclyl or a 8- to 11-membered heterobicyclyl;
   wherein L¹ is substituted with one to four moieties L²-Z and wherein L¹ is optionally further substituted;
      wherein
      L² is a single chemical bond or a spacer; and
      Z is a carrier.

Such moiety L¹ is disclosed in WO2011/140376A1 and WO2013/036847A1.

The term “C₆₋₁₈ aryl” as used for the carrier-linked relaxin prodrug comprising a moiety of formula (X) means an aromatic hydrocarbon moiety having 6 to 18 carbon atoms, preferably 6 to 10 carbon atoms, including for example phenyl, naphthyl and anthracenyl. Optionally a C₆₋₁₈ aryl may be further substituted. If such a C₆₋₁₈ aryl is connected to the rest of the moiety through an alkylene linkage, it is referred to as “arylalkyl”.

The term “C₆₋₁₈ heteroaryl” as used for the carrier-linked relaxin prodrug comprising a moiety of formula (X) refers to an aromatic moiety comprising 6 to 18, preferably 6 to 10, carbon atoms and one or more heteroatom, which is N, O or S, and which term includes for example moieties such as pyrrolyl, pyridyl, pyrimidinyl, imidazolyl, oxazolyl, isoxazolyl, thiazolyl, isothiazolyl, quinolyl, indolyl, and indenyl. Optionally a C₆₋₁₈ heteroaryl may be further substituted. If such a C₆₋₁₈ heteroaryl is connected to the rest of the moiety through an alkylene linkage, it is referred to as “heteroarylalkyl”.

Preferred embodiments for L² and Z are described above and are thus also herewith incorporated for the carrier-linked relaxin prodrug comprising the reversible linker moiety -L¹ of formula (X).

Preferably, the one to four moieties L²-Z are attached to R¹, R², R⁵, R^{5a} and/or to R⁶. If Z is a hydrogel L¹ is substituted with one moiety L²-Z which is attached to R¹, R², R⁵, R^{5a} or to R⁶.

Another aspect of the present invention is a pharmaceutical composition comprising at least one—preferably, one, two or three; even more preferably one—carrier-linked relaxin prodrug, more preferably hydrogel-linked relaxin prodrug, as described before and optionally one or more excipients.

Excipients used in parenteral compositions may be categorized as buffering agents, isotonicity modifiers, preservatives, stabilizers, anti-adsorption agents, oxidation protection agents, viscosifiers/viscosity enhancing agents, or other auxiliary agents. In some cases, these ingredients may have dual or triple functions. The one or more excipients are selected from the groups consisting of:
(i) Buffering agents: physiologically tolerated buffers to maintain pH in a desired range, such as sodium phosphate, bicarbonate, succinate, histidine, citrate and acetate, sulphate, nitrate, chloride, pyruvate. Antacids such as Mg(OH)₂ or ZnCO₃ may be also used. Buffering capacity may be adjusted to match the conditions most sensitive to pH stability
(ii) Isotonicity modifiers: to minimize pain that can result from cell damage due to osmotic pressure differences at the injection depot. Glycerin and sodium chloride are examples. Effective concentrations can be determined by osmometry using an assumed osmolality of 285-315 mOsmol/kg for serum
(iii) Preservatives and/or antimicrobials: multidose parenteral preparations require the addition of preservatives at a sufficient concentration to minimize risk of patients becoming infected upon injection and corresponding regulatory requirements have been established. Typical preservatives include m-cresol, phenol, methylparaben, ethylparaben, propylparaben, butylparaben, chlorobutanol, benzyl alcohol, phenylmercuric nitrate, thimerosol, sorbic acid, potassium sorbate, benzoic acid, chlorocresol, and benzalkonium chloride
(iv) Stabilizers: Stabilisation is achieved by strengthening of the protein-stabilising forces, by destabilisation of the denatured stater, or by direct binding of excipients to the protein. Stabilizers may be amino acids such as alanine, arginine, aspartic acid, glycine, histidine, lysine, proline, sugars such as glucose, sucrose, trehalose, polyols such as glycerol, mannitol, sorbitol, salts such as potassium phosphate, sodium sulphate, chelating agents such as EDTA, hexaphosphate, ligands such as divalent metal ions (zinc, calcium, etc.), other salts or organic molecules such as phenolic derivatives. In addition, oligomers or polymers such as cyclodextrins, dextran, dendrimers, PEG or PVP or protamine or HSA may be used
(v) Anti-adsorption agents: Mainly ionic or non-ionic surfactants or other proteins or soluble polymers are used to coat or adsorb competitively to the inner surface of the composition's container. E.g., poloxamer (Pluronic F-68), PEG dodecyl ether (Brij 35), polysorbate 20 and 80, dextran, polyethylene glycol, PEG-polyhistidine, BSA and HSA and gelatines. Chosen concentration and type of excipient depends on the effect to be avoided but typically a monolayer of surfactant is formed at the interface just above the CMC value
(vi) Lyo- and/or cryoprotectants: During freeze- or spray drying, excipients may counteract the destabilising effects caused by hydrogen bond breaking and water removal. For this purpose sugars and polyols may be used but corresponding positive effects have also been observed for surfactants, amino acids, non-aqueous solvents, and other peptides. Trehalose is particulary efficient at reducing moisture-induced aggregation and also improves thermal stability potentially caused by exposure of protein hydrophobic groups to water. Mannitol and sucrose may also be used, either as sole lyo/cryoprotectant or in combination with each other where higher ratios of mannitol:sucrose are known to enhance physical stability of a lyophilized cake. Mannitol may also be combined with trehalose. Trehalose may also be combined with sorbitol or sorbitol used as the sole protectant. Starch or starch derivatives may also be used
(vii) Oxidation protection agents: antioxidants such as ascorbic acid, ectoine, methionine, glutathione, monothioglycerol, morin, polyethylenimine (PEI), propyl gallate, vitamin E, chelating agents such aus citric acid, EDTA, hexaphosphate, thioglycolic acid
(viii) Viscosifiers or viscosity enhancers: retard settling of the particles in the vial and syringe and are used in order to facilitate mixing and resuspension of the particles and to make the suspension easier to inject (i.e., low force on the syringe plunger). Suitable viscosifiers or viscosity enhancers are, for example, carbomer viscosifiers like Carbopol 940, Carbopol Ultrez 10, cellulose derivatives like hydroxypropylmethylcellulose (hypromellose, HPMC) or diethylaminoethyl cellulose (DEAE or DEAE-C), colloidal magnesium silicate (Veegum) or sodium silicate, hydroxyapatite gel, tricalcium phosphate gel, xanthans, carrageenans like Satia gum UTC 30, aliphatic poly(hydroxy acids), such as poly(D,L- or L-lactic acid) (PLA) and poly(glycolic acid) (PGA) and their copolymers (PLGA), terpolymers of D,L-lactide, glycolide and caprolactone, poloxamers, hydrophilic poly(oxyethylene) blocks and hydrophobic poly(oxypropylene) blocks to make up a triblock of poly(oxyethylene)-poly(oxyplopylene)-poly(oxyethylene) (e.g. Pluronic®), polyetherester copolymer, such as a polyethylene glycol terephthalate/polybutylene terephthalate copolymer, sucrose acetate isobutyrate (SAIB), dcxtran or derivatives thereof, combinations of dextrans and PEG, polydimethylsiloxane, collagen, chitosan, polyvinyl alcohol (PVA) and derivatives, polyalkylimides, poly (acrylamide-co-diallyldimethyl ammonium (DADMA)), polyvinylpyrrolidone (PVP), glycosaminoglycans (GAGs) such as dermatan sulfate, chondroitin sulfate, keratan sulfate, heparin, heparan sulfate, hyaluronan, ABA triblock or AB block copolymers composed of hydrophobic A-blocks, such as polylactide (PLA) or poly(lactide-co-glycolide) (PLGA), and hydrophilic B-blocks, such as polyethylene glycol (PEG) or polyvinyl pyrrolidone. Such block copolymers as well as the abovementioned poloxamers may exhibit reverse thermal gelation behavior (fluid state at room temperature to facilitate administration and gel state above sol-gel transition temperature at body temperature after injection).
(ix) Spreading or diffusing agent: modifies the permeability of connective tissue through the hydrolysis of components of the extracellular matrix in the intrastitial space such as but not limited to hyaluronic acid, a polysaccharide found in the intercellular space of connective tissue. A spreading agent such as but not limited to hyaluronidase temporarily decreases the viscosity of the extracellular matrix and promotes diffusion of injected drugs.
(x) Other auxiliary agents: such as wetting agents, viscosity modifiers, antibiotics, hyaluronidase. Acids and bases such as hydrochloric acid and sodium hydroxide are auxiliary agents necessary for pH adjustment during manufacture

In one embodiment pharmaceutical composition comprising carrier-linked relaxin prodrug, preferably hydrogel-linked relaxin prodrug, comprises one or more preservatives and/or antimicrobials.

The pharmaceutical composition of carrier-linked relaxin prodrug, preferably hydrogel-linked relaxin prodrug, may be provided as a suspension composition or as a dry composition.

The term “suspension composition” relates to a mixture of carrier-linked relaxin prodrug, preferably hydrogel-linked relaxin prodrug, containing a water-insoluble polymer, i.e. the hydrogel carrier Z, and one or more solvents, such as water. Due to the water-insoluble polymer, the polymeric prodrug cannot dissolve and renders the prodrug in a particulate state.

“Dry composition” means that the prodrug composition is provided in a dry form. Suitable methods for drying are spray-drying and lyophilization, i.e. freeze-drying. Such dry composition of prodrug has a residual water content of a maximum of 10%, preferably less than 5% and more preferably less than 2%, determined according to Karl Fischer.

In case of dry compositions, suitable methods of drying are, for example, spray-drying and lyophilization, i.e. freeze-drying. Preferably, the pharmaceutical composition comprising hydrogel-linked relaxin prodrug is dried by lyophilization.

Another aspect of the present invention is a container comprising the carrier-linked relaxin prodrug, preferably the hydrogel-linked relaxin prodrug, or the dry or suspension form of the pharmaceutical composition comprising the carrier-linked relaxin prodrug, preferably hydrogel-linked relaxin prodrug.

Suitable containers for suspension compositions are, for example, syringes, vials, vials with stopper and seal, ampoules, and cartridges. In particular, a suspension compositions according to the present invention may be provided in a syringe.

Suitable containers for dry compositions are, for example, syringes, dual-chamber syringes, vials, vials with stopper and seal, ampoules, and cartridges. In particular, a dry composition according to the present invention may be provided in a first chamber of the dual-chamber syringe and reconstitution solution is provided in a second chamber of the dual-chamber syringe.

In one embodiment of the present invention, the dry or suspension composition of carrier-linked relaxin prodrug, preferably hydrogel-linked relaxin prodrug, is provided as a single dose, meaning that the container in which it is supplied contains one pharmaceutical dose.

In another embodiment of the present invention the dry or suspension composition comprising carrier-linked relaxin prodrug, preferably hydrogel-linked relaxin prodrug, is provided as a multiple dose composition, meaning that the container in which it is supplied contains more than one pharmaceutical dose. Such multiple dose composition of carrier-linked relaxin prodrug, preferably hydrogel-linked relaxin prodrug, can either be used for different patients in need thereof or is intended for use in one patient, wherein the remaining doses are stored after the application of the first dose until needed.

Prior to applying a dry composition of carrier-linked hydrogel, preferably hydrogel-linked relaxin prodrug, to a patient in need thereof, the dry composition is reconstituted.

Reconstitution may take place in the container in which the dry composition of hydrogel-linked relaxin prodrug is provided, such as in a vial, vial with stopper and seal, syringe, dual-chamber syringe, ampoule, and cartridge.

Reconstitution is done by adding a predefined amount of reconstitution solution to the dry composition. Reconstitution solutions are sterile liquids, such as water or buffer, which may contain further additives, such as preservatives and/or antimicrobials, such as, for example, benzyl alcohol and cresol. Preferably, the reconstitution solution is sterile water.

A further aspect is a method of preparing a reconstituted composition comprising a therapeutically effective amount of carrier-linked relaxin prodrug, preferably hydrogel-linked relaxin prodrug, of the present invention, and optionally one or more pharmaceutically acceptable excipients the method comprising the step of
contacting the dry pharmaceutical composition with a reconstitution solution.

Another aspect is a reconstituted composition comprising a therapeutically effective amount of carrier-linked relaxin prodrug, preferably hydrogel-linked relaxin prodrug, of the present invention, and optionally one or more pharmaceutically acceptable excipients.

Another aspect of the present invention is the method of manufacturing a suspension composition of carrier-linked relaxin prodrug, preferably hydrogel-linked relaxin prodrug. In one embodiment, such suspension composition is made by
(i) admixing the carrier-linked relaxin prodrug, preferably the hydrogel-linked relaxin prodrug, with one or more excipients,
(ii) transferring amounts equivalent to single or multiple doses into a suitable container, and
(iii) sealing the container.

Suitable containers are syringes, vials, vials with stopper and seal, ampoules, and cartridges.

Another aspect of the present invention is the method of manufacturing a dry composition of carrier-linked relaxin prodrug, preferably hydrogel-linked relaxin prodrug. In one embodiment, such dry composition is made by
(i) admixing the carrier-linked relaxin hydrogel, preferably hydrogel-linked relaxin prodrug, with one or more excipients,
(ii) transferring amounts equivalent to single or multiple doses into a suitable container,
(iii) drying the composition in said container, and
(iv) sealing the container.

Alternatively, the method comprises the steps of
(i) transferring amounts equivalent to single or multiple doses of carrier-linked relaxin prodrug, preferably hydrogel-linked relaxin prodrug, into a suitable container,
(ii) adding one or more excipients to the container,
(iii) drying the composition in said container, and
(iv) sealing the container.

Suitable containers are syringes, dual-chamber syringes, vials, vials with stopper and seal, ampoules, and cartridges.

“Sealing a container” means that the container is closed in such way that it is airtight, allowing no gas exchange between the outside and the inside and maintaining sterility, if the content of the container is sterile.

Another aspect is a kit of parts for a dry composition according to the present invention. When the administration device is simply a hypodermic syringe then the kit may comprise the syringe, a needle and a container comprising the dry carrier-linked relaxin prodrug, preferably the dry hydrogel-linked relaxin prodrug, composition for use with the syringe and a second container comprising the reconstitution solution. In more preferred embodiments, the injection device is other than a simple hypodermic syringe and so the separate container with reconstituted carrier-linked relaxin prodrug, preferably hydrogel-linked relaxin prodrug, is adapted to engage with the injection device such that in use the suspension composition in the container is in fluid connection with the outlet of the injection device. Examples of administration devices include but are not limited to hypodermic syringes and pen injector devices. Particularly preferred injection devices are syringes suitable for subcutaneous injection.

A preferred kit of parts for a dry composition comprises a needle and a container containing the composition according to the present invention and optionally further containing a reconstitution solution, the container being adapted for use with the needle. Preferably, the container is a dual-chamber syringe.

Another aspect is a kit of parts for a suspension composition according to the present invention. When the administration device is simply a hypodermic syringe then the kit may comprise a container with the suspension composition and a needle for use with the container.

In another aspect, the invention provides a cartridge containing a composition of carrier-linked relaxin prodrug, preferably hydrogel-linked relaxin prodrug, whether in dry or suspension form, as hereinbefore described for use with a syringe suitable for subcutaneous injection. The cartridge may contain a single dose or a multiplicity of doses of carrier-linked relaxin prodrug, preferably hydrogel-linked relaxin prodrug.

Another aspect of the present invention is a carrier-linked relaxin prodrug, preferably a hydrogel-linked relaxin prodrug of the present invention or a pharmaceutically acceptable salt thereof or a pharmaceutical composition comprising at least one of such carrier-linked relaxin prodrug, preferably at least one of such hydrogel-linked relaxin prodrug, for use as a medicament.

Another aspect of the present invention is the carrier-linked relaxin prodrug, preferably the hydrogel-linked relaxin prodrug, of the present invention or the pharmaceutical composition comprising the carrier-linked relaxin prodrug, preferably the hydrogel-linked relaxin prodrug, for use in a method of treatment of a disease which can be treated with relaxin.

In one embodiment, said disease is heart failure. Heart failure is defined as the inability of the cardiac pump to move blood as needed to provide for the metabolic needs of body tissue. Heart failure may be acute or chronic and accordingly said disease is acute or chronic heart failure.

In another embodiment, said disease is a kidney disease.

In another embodiment, said disease is fibrosis, in particular fibrosis of the heart, lungs, kidney and/or liver.

In another embodiment, said disease is pulmonary hypertension, in particular pulmonary arterial hypertension.

In another embodiment, said disease is atherosclerosis.

In another embodiment, said disease is Type 1 or Type 2 diabetes.

In another embodiment, said disease is a coronary artery disease.

In another embodiment, said disease is scleroderma.

In another embodiment, said disease is stroke.

In another embodiment, said disease is diastolic dysfunction.

In another embodiment, said disease is familial hypercholesterolemia.

In another embodiment, said disease is isolated systolic hypertension, primary hypertension or secondary hypertension.

In another embodiment, said disease is left ventricular hypertrophy.

In another embodiment, said disease is arterial stiffness associated with long-term tobacco smoking, obesity or age.

In another embodiment, said disease is systemic lupus erythematosus.

In another embodiment, said disease is preeclampsia.

In another embodiment, said disease is hypercholesterolemia.

Another aspect of the present invention is the use of the carrier-linked relaxin prodrug, preferably the hydrogel-linked relaxin prodrug, or a pharmaceutically acceptable salt thereof or a pharmaceutical composition comprising carrier-linked relaxin prodrug, preferably hydrogel-linked relaxin prodrug, for the manufacture of a medicament for treating one or more disease(s) which can be treated with relaxin.

In one embodiment, said disease is heart failure.

In another embodiment, said disease is a kidney disease.

In another embodiment, said disease is fibrosis, in particular fibrosis of the heart, lungs, kidney and/or liver.

In another embodiment, said disease is pulmonary hypertension, in particular pulmonary arterial hypertension.

In another embodiment, said disease is atherosclerosis.

In another embodiment, said disease is Type 1 or Type 2 diabetes.

In another embodiment, said disease is a coronary artery disease.

In another embodiment, said disease is scleroderma.

In another embodiment, said disease is stroke.

In another embodiment, said disease is diastolic dysfunction.

In another embodiment, said disease is familial hypercholesterolemia.

In another embodiment, said disease is isolated systolic hypertension, primary hypertension or secondary hypertension.

In another embodiment, said disease is left ventricular hypertrophy.

In another embodiment, said disease is arterial stiffness associated with long-term tobacco smoking, obesity or age.

In another embodiment, said disease is systemic lupus erythematosus.

In another embodiment, said disease is preeclampsia.

In another embodiment, said disease is hypercholesterolemia.

A further aspect of the present invention is a method of treating, controlling, delaying or preventing in a mammalian patient, preferably a human patient, in need of the treatment of one or more diseases which can be treated with relaxin, comprising the step of administering to said patient in need thereof a therapeutically effective amount of carrier-linked relaxin prodrug, preferably hydrogel-linked relaxin prodrug, or a pharmaceutically acceptable salt thereof or a pharmaceutical composition comprising carrier-linked relaxin prodrug, preferably hydrogel-linked relaxin prodrug, of the present invention.

An additional aspect of the present invention relates to the way of administration of a carrier-linked relaxin prodrug, preferably hydrogel-linked relaxin prodrug, or a reconstituted or suspension pharmaceutical composition of carrier-linked relaxin prodrug, preferably hydrogel-linked relaxin prodrug, which can be administered via topical, enteral or parenteral administration and by methods of external application, injection or infusion, including intraarticular, intradermal, subcutaneous, intramuscular, intravenous, intraosseous, and intraperitoneal, intrathecal, intracapsular, intraorbital, intravitreal, intratympanic, intravesical, intracardiac, transtracheal, subcuticular, subcapsular, subarachnoid, intraspinal, intraventricular and intrasternal.

In a preferred embodiment, the present invention relates to a carrier-linked relaxin prodrug, preferably a hydrogel-linked relaxin prodrug, or pharmaceutically acceptable salt thereof or a pharmaceutical composition of the present invention, for use in the treatment of heart failure via subcutaneous injection.

In a preferred embodiment, the present invention relates to a carrier-linked relaxin hydrogel, preferably a hydrogel-linked relaxin prodrug, or pharmaceutically acceptable salt thereof or a pharmaceutical composition of the present invention, for use in the treatment of a kidney disease via subcutaneous injection.

In a preferred embodiment, the present invention relates to a carrier-linked relaxin prodrug, preferably a hydrogel-linked relaxin prodrug, or pharmaceutically acceptable salt thereof or a pharmaceutical composition of the present invention, for use in the treatment of fibrosis, in particular fibrosis of the heart, lungs, kidney and/or liver, via subcutaneous injection.

In a preferred embodiment, the present invention relates to a carrier-linked relaxin prodrug, preferably a hydrogel-linked relaxin prodrug, or pharmaceutically acceptable salt thereof or a pharmaceutical composition of the present invention, for use in the treatment of pulmonary hypertension, in particular pulmonary arterial hypertension, via subcutaneous injection.

FIG. 1a: Overview of the A- and B-chain and the location of the two inter- and one intra-molecular disulfide bonds of RLN2

FIG. 1b: Overview of the A- and B-chain and the location of the two inter- and one intra-molecular disulfide bonds of RLN3.

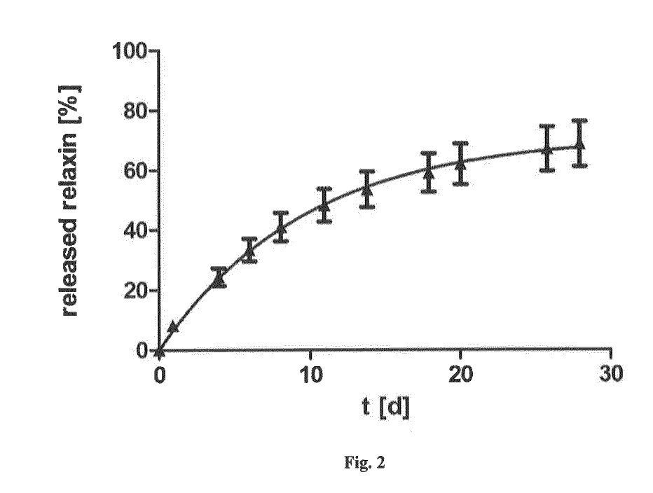

FIG. 2: Plot of relaxin release from compound 7 at pH 7.4 and 37° C. against incubation time

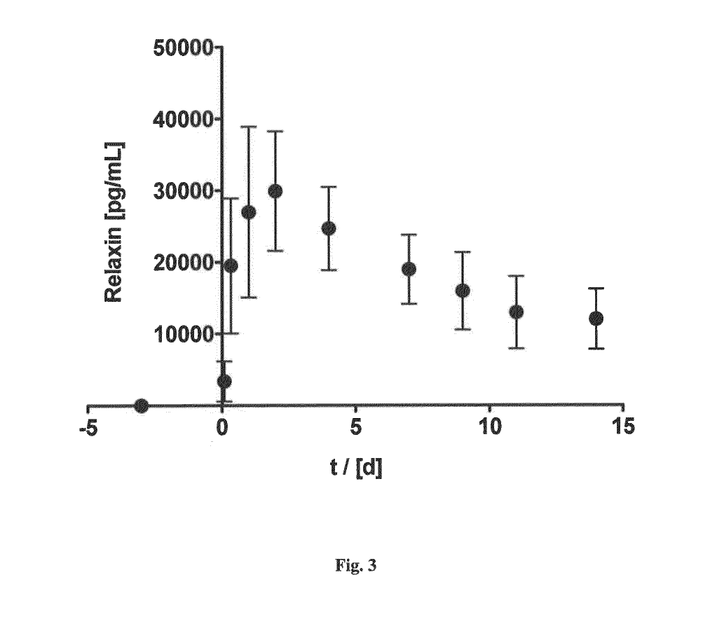

FIG. 3: Pharmacokinetics of compound 7 shown as mean relaxin plasma levels

### EXAMPLES

### Materials and Methods

Relaxin H2 (human) trifluoroacetate salt was obtained from Bachem AG, Bubendorf, Switzerland.

Amino 4-arm PEG 5 kDa was obtained from JenKem Technology, Beijing, P. R. China.

N-(3-maleimidopropyl)-21-amino-4,7,10,13,16,19-hexaoxa-heneicosanoic acid NHS ester (Mal-PEG6-NHS) was obtained from Celares GmbH, Berlin, Germany.

HATU, N-cyclohexyl-carbodiimide-N′-methyl polystyrene, and amino acids were from Merck Biosciences GmbH, Schwalbach/Ts, Germany, if not stated otherwise. Fmoc(NMe)-Asp(OtBu)-OH was obtained from Bachem AG, Bubendorf, Switzerland. S-Trityl-6-mercaptohexanoic acid was purchased from Polypeptide, Strasbourg, France. Amino acids used were of L configuration if not stated otherwise.

40 kDa 4-arm PEG maleimide is available from NOF Corporation, Tokyo, Japan and has the following structure:

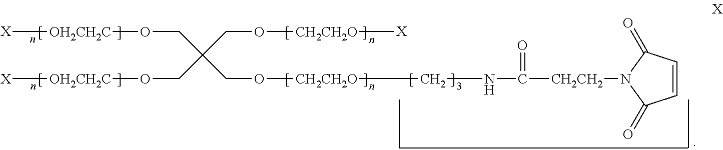

All other chemicals were from Sigma-ALDRICH Chemie GmbH, Taufkirchen, Germany.

### RP-HPLC Purification:

RP-HPLC was done on a 100×20 mm or 100×40 mm C18 ReproSil-Pur 300 ODS-3 5μ column (Dr. Maisch, Ammerbuch, Germany) connected to a Waters 600 HPLC System and Waters 2487 Absorbance detector. Linear gradients of solution A (0.1% TFA in H₂O) and solution B (0.1% TFA in acetonitrile) were used. HPLC fractions containing product were lyophilized.

### Flash Chromatography

Flash chromatography purifications were performed on an Isolera One system from Biotage AB, Sweden, using Biotage KP-Sil silica cartridges and n-heptane and ethyl acetate as eluents. Products were detected at 254 nm.

For hydrogel beads, syringes equipped with polypropylene frits were used as reaction vessels or for washing steps.

### Analytical Methods

Analytical ultra-performance LC (UPLC) was performed on a Waters Acquity system equipped with a Waters BEH300 C18 column (2.1×50 mm, 1.7 μm particle size) coupled to a LTQ Orbitrap Discovery mass spectrometer from Thermo Scientific.

MS of PEG products showed a series of (CH₂CH₂O)ₙ moieties due to polydispersity of PEG staring materials. For easier interpretation only one single representative m/z signal is given in the examples. MS of relaxin conjugates are reported for representative isotopes and refer to the four-proton adducts [M+4H]⁴⁺.

Size exclusion chromatography (SEC) was performed using an Amersham Bioscience AEKTAbasic system equipped with a Superdex200 5/150 GL column (Amersham Bioscience/GE Healthcare) equipped with a 0.45 μm inlet filter, if not stated otherwise. 20 mM sodium phosphate, 140 mM NaCl, pH 7.4, was used as mobile phase.

### Example 1

### Synthesis of Backbone Reagent 1g

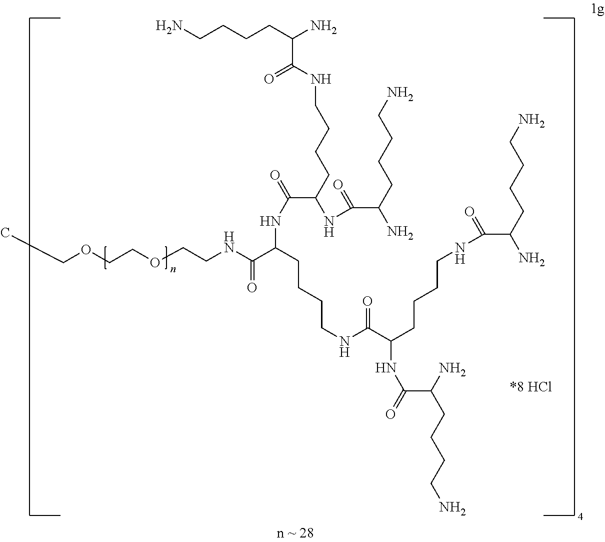

Backbone reagent 1 g was synthesized from amino 4-arm PEG5000 1 a according to following scheme:

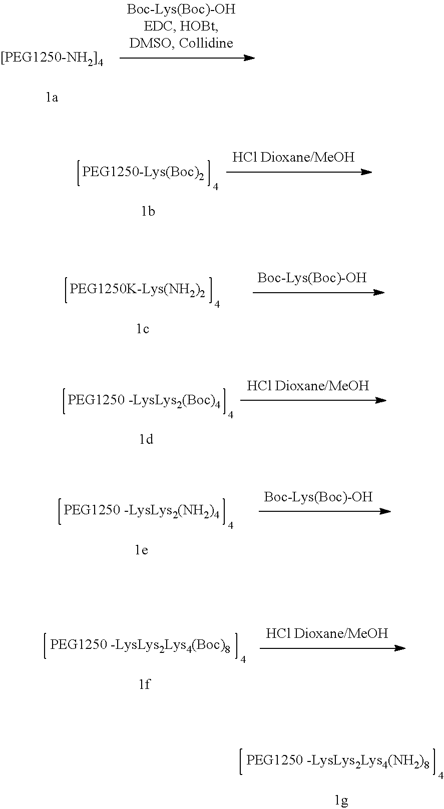

For synthesis of compound 1b, amino 4-arm PEG5000 1a (MW ca. 5200 g/mol, 5.20 g, 1.00 mmol, HCl salt) was dissolved in 20 mL of DMSO (anhydrous). Boc-Lys(Boc)-OH (2.17 g, 6.25 mmol) in 5 mL of DMSO (anhydrous), EDC HCl (1.15 g, 6.00 mmol), HOBt H₂O (0.96 g, 6.25 mmol), and collidine (5.20 mL, 40 mmol) were added. The reaction mixture was stirred for 30 min at RT.

The reaction mixture was diluted with 1200 mL of dichloromethane and washed with 600 mL of 0.1 N H₂SO₄ (2×), brine (1×), 0.1 M NaOH (2×), and 1/I (v/v) brine/water (4×). Aqueous layers were reextracted with 500 mL of DCM. Organic phases were dried over Na₂SO₄, filtered and evaporated to give 6.3 g of crude product 1b as colorless oil. Compound 1b was purified by RP-HPLC.

Yield 3.85 g (59%) colorless glassy product 1b.

MS: m/z 1294.4=[M+5H]⁵⁺ (calculated=1294.6).

Compound 1c was obtained by stirring of 3.40 g of compound 1b (0.521 mmol) in 5 mL of methanol and 9 mL of 4 N HCl in dioxane at RT for 15 min. Volatiles were removed in vacuo. The product was used in the the next step without further purification.

MS: m/z 1151.9=[M+5H]⁵⁺ (calculated=1152.0).

For synthesis of compound 1d, 3.26 g of compound 1c (0.54 mmol) were dissolved in 15 mL of DMSO (anhydrous). 2.99 g Boc-Lys(Boc)-OH (8.64 mmol) in 15 mL DMSO (anhydrous), 1.55 g EDC HCl (8.1 mmol), 1.24 g HOBt H₂O (8.1 mmol), and 5.62 mL of collidine (43 mmol) were added. The reaction mixture was stirred for 30 min at RT. Reaction mixture was diluted with 800 mL DCM and washed with 400 mL of 0.1 N H₂SO₄ (2×), brine (1×), 0.1 M NaOH (2×), and 1/1 (v/v) brine/water (4×). Aqueous layers were reextracted with 800 mL of DCM. Organic phases were dried with Na₂SO₄, filtered and evaporated to give a glassy crude product. Product was dissolved in DCM and precipitated with cooled (−18° C.) diethylether. This procedure was repeated twice and the precipitate was dried in vacuo.

Yield: 4.01 g (89%) colorless glassy product 1d, which was used in the next step without further purification.

MS: m/z 1405.4=[M+6H]⁶⁺ (calculated=1405.4).

Compound 1e was obtained by stirring a solution of compound 1d (3.96 g, 0.47 mmol) in 7 mL of methanol and 20 mL of 4 N HCl in dioxane at RT for 15 min. Volatiles were removed in vacuo. The product was used in the the next step without further purification.

MS: m/z 969.6=[M+7H]⁷⁺ (calculated=969.7).

For the synthesis of compound 1f, compound 1e (3.55 g, 0.48 mmol) was dissolved in 20 mL of DMSO (anhydrous). Boc-Lys(Boc)-OH (5.32 g, 15.4 mmol) in 18.8 mL of DMSO (anhydrous), EDC HCl (2.76 g, 14.4 mmol), HOBt-H₂O (2.20 g, 14.4 mmol), and 10.0 mL of collidine (76.8 mmol) were added. The reaction mixture was stirred for 60 min at RT.

The reaction mixture was diluted with 800 mL of DCM and washed with 400 mL of 0.1 N H₂SO₄ (2×), brine (1×), 0.1 M NaOH (2×), and 1/1 (v/v) brine/water (4×). Aqueous layers were reextracted with 800 mL of DCM. Organic phases were dried over Na₂SO₄, filtered and evaporated to give crude product if as colorless oil.

Product was dissolved in DCM and precipitated with cooled (−18° C.) diethylther. This step was repeated twice and the precipitate was dried in vacuo.

Yield 4.72 g (82%) colourless glassy product 1f which was used in the next step without further purification.

MS: m/z 1505.3=[M+8H]⁸⁺ (calculated=1505.4).

Backbone reagent 1g was obtained by stirring a solution of compound if (MW ca 12035 g/mol, 4.72 g, 0.39 mmol) in 20 mL of methanol and 40 mL of 4 N HCl in dioxane at RT for 30 min. Volatiles were removed in vacuo.

Yield 3.91 g (100%), glassy product backbone reagent 1g.

MS: m/z 977.2=[M+9H]⁹⁺ (calculated=977.4).

### Alternative Synthetic Route for 1g

For synthesis of compound 1b, to a suspension of 4-Arm-PEG5000 tetraamine (1a) (50.0 g, 10.0 mmol) in 250 mL of iPrOH (anhydrous), boc-Lys(boc)-OSu (26.6 g, 60.0 mmol) and DIEA (20.9 mL, 120 mmol) were added at 45° C. and the mixture was stirred for 30 min.

Subsequently, n-propylamine (2.48 mL, 30.0 mmol) was added. After 5 min the solution was diluted with 1000 mL of MTBE and stored overnight at −20° C. without stirring. Approximately 500 mL of the supernatant were decanted off and discarded. 300 mL of cold MTBE were added and after 1 min shaking the product was collected by filtration through a glass filter and washed with 500 mL of cold MTBE. The product was dried in vacuo for 16 h.

Yield: 65.6 g (74%) 1b as a white lumpy solid

MS: m/z 937.4=[M+7H]⁷⁺ (calculated=937.6).

Compound 1c was obtained by stirring of compound 1b from the previous step (48.8 g, 7.44 mmol) in 156 mL of 2-propanol at 40° C. A mixture of 196 mL of 2-propanol and 78.3 mL of acetylchloride was added under stirring within 1-2 min. The solution was stirred at 40° C. for 30 min and cooled to −30° C. overnight without stirring. 100 mL of cold MTBE were added, the suspension was shaken for 1 min and cooled for 1 h at −30° C. The product was collected by filtration through a glass filter and washed with 200 mL of cold MTBE. The product was dried in vacuo for 16 h.

Yield: 38.9 g (86%) 1c as a white powder

MS: m/z 960.1=[M+6H]⁶⁺ (calculated=960.2).

For synthesis of compound 1d, to a suspension of c1 from the previous step (19.0 g, 3.14 mmol) in 80 ml 2-propanol boc-Lys(boc)-OSu (16.7 g, 37.7 mmol) and DIEA (13.1 mL, 75.4 mmol) were added at 45° C. and the mixture was stirred for 30 min at 45° C. Subsequently, n-propylamine (1.56 mL, 18.9 mmol) was added. After 5 min the solution was precipitated with 600 mL of cold MTBE and centrifuged (3000 min⁻¹, 1 min) The precipitate was dried in vacuo for 1 h and dissolved in 400 mL THF. 200 mL of diethyl ether were added and the product was cooled to −30° C. for 16 h without stirring. The suspension was filtered through a glass filter and washed with 300 mL cold MTBE. The product was dried in vacuo for 16 h.

Yield: 21.0 g (80%) 1d as a white solid

MS: m/z 1405.4=[M+6H]⁶⁺ (calculated=1405.4).

Compound 1e was obtained by dissolving compound 1d from the previous step (15.6 g, 1.86 mmol) in in 3 N HCl in methanol (81 mL, 243 mmol) and stirring for 90 min at 40° C. 200 mL of MeOH and 700 mL of iPrOH were added and the mixture was stored for 2 h at −30° C. For completeness of crystallization, 100 mL of MTBE were added and the suspension was stored at −30° C. overnight. 250 mL of cold MTBE were added, the suspension was shaken for 1 min and filtered through a glass filter and washed with 100 mL of cold MTBE.

The product was dried in vacuo.

Yield: 13.2 g (96%) 1e as a white powder

MS: m/z 679.1=[M+10H]¹⁰⁺ (calculated=679.1).

For the synthesis of compound 1f, to a suspension of 1e from the previous step, (8.22 g, 1.12 mmol) in 165 ml 2-propanol boc-Lys(boc)-OSu (11.9 g, 26.8 mmol) and DIEA (9.34 mL, 53.6 mmol) were added at 45° C. and the mixture was stirred for 30 min. Subsequently, n-propylamine (1.47 mL, 17.9 mmol) was added. After 5 min the solution was cooled to −18° C. for 2 h, then 165 mL of cold MTBE were added, the suspension was shaken for 1 min and filtered through a glass filter. Subsequently, the filter cake was washed with 4×200 mL of cold MTBE/iPrOH 4:1 and 1×200 mL of cold MTBE. The product was dried in vacuo for 16 h.

Yield: 12.8 g, MW (90%) If as a pale yellow lumpy solid

MS: m/z 1505.3=[M+8H]⁸⁺ (calculated=1505.4).

Backbone reagent 1g was obtained by dissolving 4ArmPEG5 kDa(-LysLys₂Lys₄(boc)s)₄ (If) (15.5 g, 1.29 mmol) in 30 mL of MeOH and cooling to 0° C. 4 N HCl in dioxane (120 mL, 480 mmol, cooled to 0° C.) was added within 3 min and the ice bath was removed. After 20 min, 3 N HCl in methanol (200 mL, 600 mmol, cooled to 0° C.) was added within 15 min and the solution was stirred for 10 min at room temperature. The product solution was precipitated with 480 mL of cold MTBE and centrifuged at 3000 rpm for 1 min. The precipitate was dried in vacuo for 1 h and redissolved in 90 mL of MeOH, precipitated with 240 mL of cold MTBE and the suspension was centrifuged at 3000 rpm for 1 min. The product 1g was dried in vacuo

Yield: 11.5 g (89%) as pale yellow flakes.

MS: m/z 1104.9=[M+8H]⁸⁺ (calculated=1104.9).

### Example 2

### Synthesis of Crosslinker Reagent 2d

Crosslinker reagent 2d was prepared from adipic acid mono benzyl ester (English, Arthur R. et al., Journal of Medicinal Chemistry, 1990, 33(1), 344-347) and PEG2000 according to the following scheme:

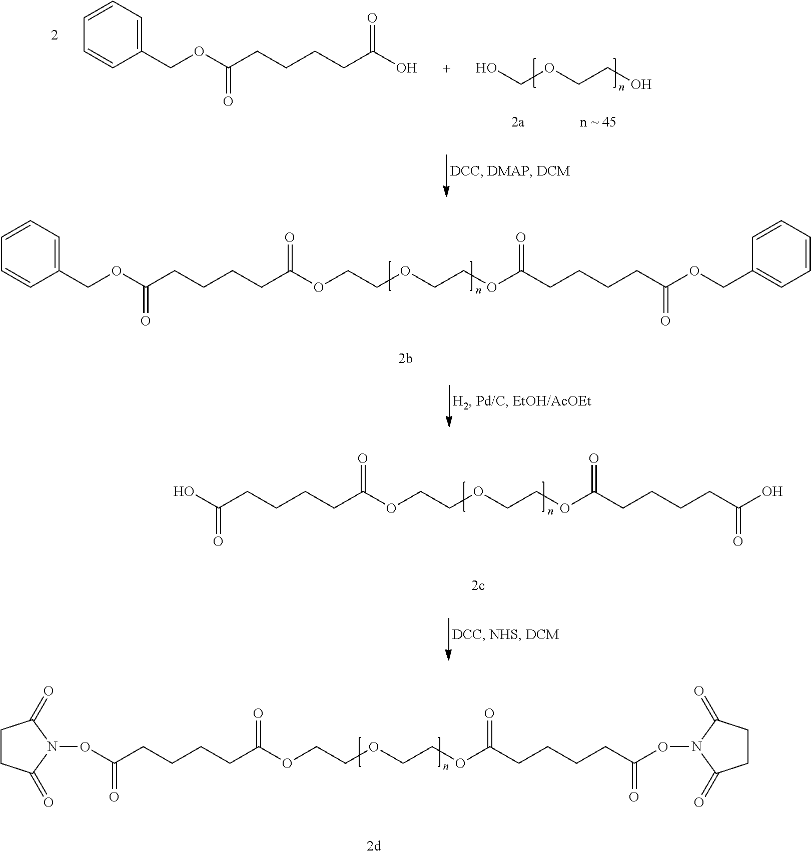

A solution of PEG 2000 (2a) (11.0 g, 5.5 mmol) and benzyl adipate half-ester (4.8 g, 20.6 mmol) in dichloromethane (90.0 mL) was cooled to 0° C. Dicyclohexylcarbodiimide (4.47 g, 21.7 mmol) was added followed by a catalytic amount of DMAP (5 mg) and the solution was stirred and allowed to reach room temperature overnight (12 h). The flask was stored at +4° C. for 5 h. The solid was filtered and the solvent completely removed by destillation in vacuo. The residue was dissolved in 1000 mL 1/1(v/v) diethyl ether/ethyl acetate and stored at RT for 2 hours while a small amount of a flaky solid was formed. The solid was removed by filtration through a pad of Celite®. The solution was stored in a tightly closed flask at −30° C. in the freezer for 12 h until crystallisation was complete. The crystalline product was filtered through a glass frit and washed with cooled diethyl ether (−30° C.). The filter cake was dried in vacuo. Yield: 11.6 g (86%) 2b as a colorless solid. The product was used without further purification in the next step.

MS: m/z 813.1=[M+3H]³⁺ (calculated=813.3)

In a 500 mL glass autoclave PEG2000-bis-adipic acid-bis-benzyl ester 2b (13.3 g, 5.5 mmol) was dissolved in ethyl acetate (180 mL) and 10% Palladium on charcoal (0.4 g) was added. The solution was hydrogenated at 6 bar, 40° C. until consumption of hydrogen had ceased (5-12 h). Catalyst was removed by filtration through a pad of Celite® and the solvent was evaporated in vacuo. Yield: 12.3 g (quantitative) 2c as yellowish oil. The product was used without further purification in the next step.

MS: m/z 753.1=[M+3H]³⁺ (calculated=753.2)

A solution of PEG2000-bis-adipic acid half ester 2c (9.43 g, 4.18 mmol), N-hydroxysuccinimide (1.92 g, 16.7 mmol) and dicyclohexylcarbodiimide (3.44 g, 16.7 mmol) in 75 mL of DCM (anhydrous) was stirred over night at room temperature. The reaction mixture was cooled to 0° C. and precipitate was filtered off. DCM was evaporated and the residue was recystallized from THF.

Yield: 8.73 g (85%) crosslinker reagent 2d as colorless solid.

MS: m/z 817.8=[M+3H]³⁺ (calculated=817.9 g/mol).

### Synthesis of 2e

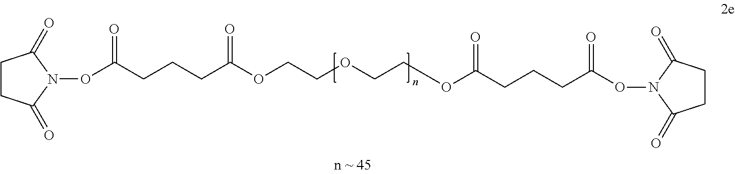

2e was synthesized as described for 2d except for the use of glutaric acid instead of adipic acid

MS: m/z 764.4=[M+3H]³⁺ (calculated=764.5).

### Synthesis of 2f

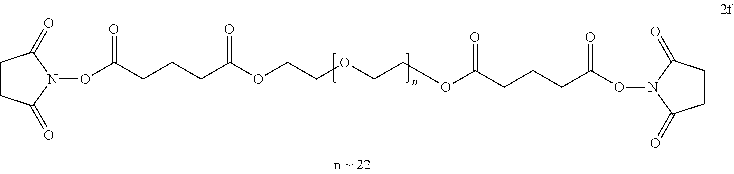

2f was synthesized as described for 2e except for the use of PEG 1000 instead of PEG2000 MS: m/z 727.4=[M+2H]²⁺ (calculated=727.4).

### Example 3

### Preparation of Hydrogel Beads (3a), (3b), and (3c) Containing Free Amino Groups

A solution of 800 mg 1g and 2430 mg 2d in 19.8 g DMSO was added to a solution of 269 mg Cithrol DPHS (Croda International Plc) in 100 mL undecane. The mixture was stirred at 620 rpm with a custom metal stirrer for 10 min at 25° C. to form a suspension. 3.6 mL N,N,N′,N′-tetramethyl-ethylene-diamine was added to effect polymerization. After 16 h, 5.5 mL of acetic acid were added and then after 10 min 100 mL of a 15 wt % solution of sodium chloride in water were added. After 10 min, the stirrer was stopped and the aqueous phase was drained after 2 h.

For bead size fractionation, the water-hydrogel suspension was wet-sieved on 100, 75, 63, 50, and 40 μm mesh steel sieves. Bead fractions that were retained on the 40, 50, and 63 μm sieves were washed 3 times with 0.1% acetic acid in, 10 times with ethanol and dried for 16 h at 0.1 mbar to give 3a as a white powder. 40 μm fraction: 320 mg, 50 μm fraction: 540 mg, 63 μm fraction: 720 mg.

3b was prepared as described for 3a except for the use of 1000 mg 1g, 3125 mg 2e, 25.3 g DMSO, 260 mg Cithrol DPHS, 100 mL heptane instead of undecane, and 4.5 ml TMEDA. For workup, 6.9 ml acetic acid were added. 3b was obtained as a white powder, 40 μm fraction: 538 mg, 50 μm fraction: 904 mg, 63 μm fraction: 607 mg. 3c was prepared as described for 3a except for the use of 1000 mg 1g, 2145 mg 2f, 19.3 g DMSO, 199 mg Cithrol DPHS, 100 mL heptane instead of undecane, and 4.5 ml TMEDA. For workup, 6.9 ml acetic acid were added. 3c was obtained as a white powder, 40 μm fraction: 133 mg, 50 μm fraction: 370 mg, 63 μm fraction: 714 mg.

Amino group content of hydrogel was determined by conjugation of a fmoc-amino acid to the free amino groups on the hydrogel and subsequent fmoc-determination as described by Gude, M., J. Ryf, et al. (2002) Letters in Peptide Science 9(4): 203-206.

The amino group content of 3a, 3b and 3c was determined to be between 0.074 and 0.137 mmol/g.

### Example 4

### Preparation of Maleimide Functionalized Hydrogel Beads (4) and Determination of Maleimide Substitution

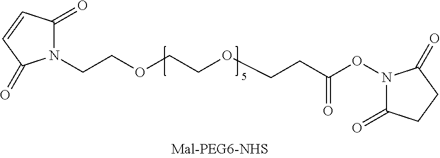

800 mg dry hydrogel 3a (110 mol amino groups) was filled into 2 syringes equipped with filter flits. The hydrogel was re-suspended and washed 10 times in NMP/1% n-propylamine and 5 times with DMSO. The solvent was expelled and 2.74 mL of a 24 mg/mL solution of Mal-PEG6-NHS in DMSO was drawn into each of the two syringes (2 eq, 219 μmol). The syringes were incubated for 90 min at room temperature, washed 5 times with DMSO and 10 times with sodium succinate buffer (pH 3.0, 20 mM; 1 mM EDTA, 0.01% Tween-20). The buffer was expelled, the hydrogel pellets transferred to a sample vial and filled-up to 20 mL with sodium succinate buffer (pH 3.0, 20 mM; 1 mM EDTA, 0.01% Tween-20).

For determination of the maleimide content, an aliquot of hydrogel beads 4 was reacted with Fmoc-L-cysteine. The amount of Fmoc on the hydrogel was quantified photometrically in the supernatant after cleavage of the protecting group with DBU/DMF. The maleimide content of 4 was determined to be 0.137 mmol/g.

### Example 5

### Synthesis of Linker Reagent 5f

Linker reagent 5f was synthesized according to the following scheme:

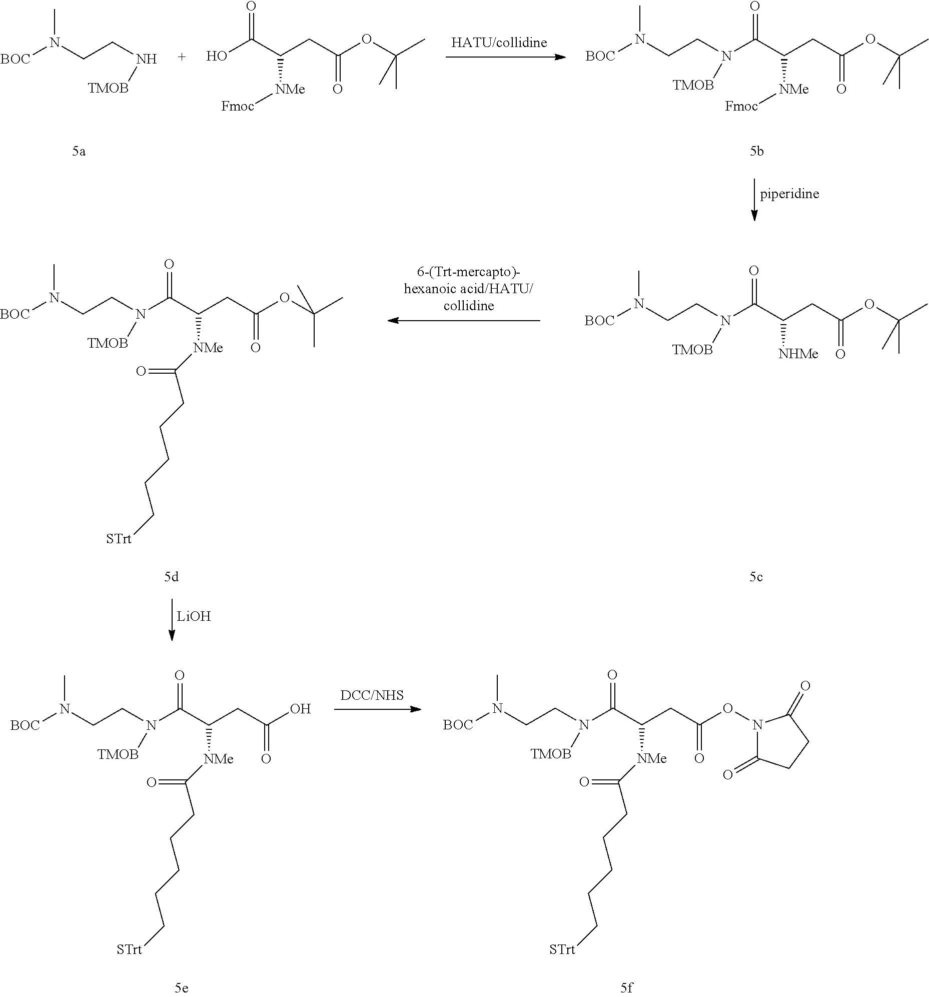

To a cooled (0° C.) solution of N-Methyl-N-boc-ethylendiamine (0.5 mL, 2.79 mmol) and NaCNBH₃ (140 mg, 2.23 mmol) in MeOH (10 mL) and acetic acid (0.5 mL) was added a solution of 2,4,6-trimethoxybenzaldehyde (0.547 mg, 2.79 mmol) in EtOH (10 mL). The mixture was stirred at RT for 2 h, acidified with 2 M HCl (1 mL) and neutralized with saturated aqueous Na₂CO₃ (50 mL). Evaporation of all volatiles, DCM extraction of the resulting aqueous slurry and concentration of the organic fractions yielded N-Methyl-N-boc-N′-tmob-ethylendiamine (5a) as a crude oil which was purified by RP-HPLC.

Yield: 593 mg (1.52 mmol)

MS: m/z 377.35=[M+Na]⁺, (calculated=377.14).

N-Fmoc-N-Me-Asp(OtBu)-OH (225 mg, 0.529 mmol) was dissolved in DMF (3 mL) and 5a (300 mg, 0.847 mmol), HATU (201 mg, 0.529 mmol), and collidine (0.48 mL, 3.70 mmol) were added. The mixture was stirred at RT for 2 h to yield 5b. For fmoc deprotection, piperidine (0.22 mL, 2.16 mmol) was added and stirring was continued for 1 h. Acetic acid (1 mL) was added, and 5c was purified by RP-HLPC.

Yield: 285 mg (0.436 mmol as TFA salt)

MS: m/z 562.54=[M+Na]⁺, (calculated=562.67).

6-Tritylmercaptohexanoic acid (0.847 g, 2.17 mmol) was dissolved in anhydrous DMF (7 mL). HATU (0.825 g, 2.17 mmol), and collidine (0.8 mL, 6.1 mmol) and 5c (0.78 g, 1.44 mmol) were added. The reaction mixture was stirred for 60 min at RT, acidified with AcOH (1 mL) and purified by RP-HPLC. Product fractions were neutralized with saturated aqueous NaHCO₃ and concentrated. The remaining aqueous phase was extracted with DCM and 5d was isolated upon evaporation of the solvent.

Yield: 1.4 g (94%)

MS: m/z 934.7=[M+Na]⁺, (calculated=934.5).

To a solution of 5d (1.40 mg, 1.53 mmol) in MeOH (12 mL) and H₂O (2 mL) was added LiOH (250 mg, 10.4 mmol) and the reaction mixture was stirred for 14 h at 70° C. The mixture was acidified with AcOH (0.8 mL) and 5e was purified by RP-HPLC. Product fractions were neutralized with saturated aqueous NaHCO₃ and concentrated. The aqueous phase was extracted with DCM and 5e was isolated upon evaporation of the solvent.

Yield: 780 mg (60%)

MS: m/z 878.8=[M+Na]⁺, (calculated=878.40).

To a solution of 5e (170 mg, 0.198 mmol) in anhydrous DCM (4 mL) were added DCC (123 mg, 0.59 mmol) and N-hydroxy-succinimide (114 mg, 0.99 mmol), and the reaction mixture was stirred at RT for 1 h. The mixture was filtered, and the filtrate was acidified with 0.5 mL AcOH and 5f purified by RP-HPLC. Product fractions were neutralized with saturated aqueous NaHCO₃ and concentrated. The remaining aqueous phase was extracted with DCM and 5f was isolated upon evaporation of the solvent.

Yield: 154 mg (0.161 mmol)

MS: m/z 953.4=[M+H]⁺, (calculated=953.43).

Alternatively, linker reagent 5f was synthesized according to the following procedure: Alternative reaction scheme:

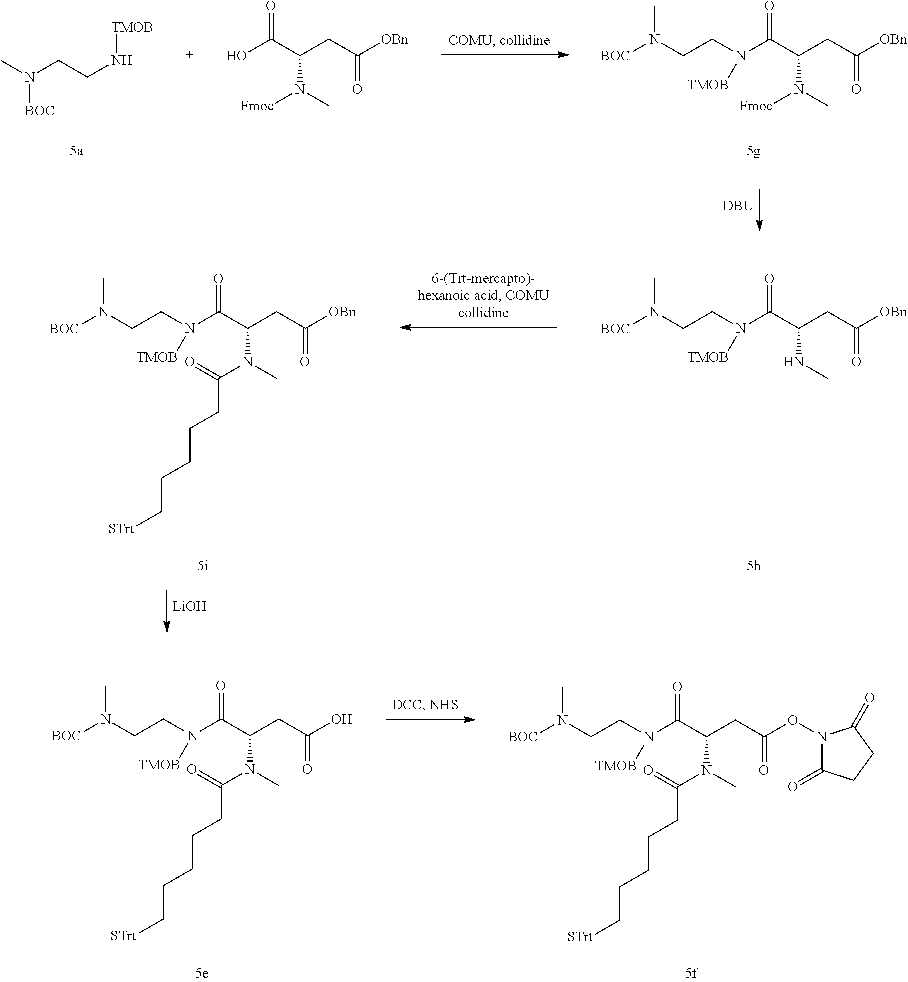

To a solution of N-Methyl-N-boc-ethylenediamine (2 g, 11.48 mmol) and NaCNBH₃ (819 mg, 12.63 mmol) in MeOH (20 mL) was added 2,4,6-trimethoxybenzaldehyde (2.08 mg, 10.61 mmol) portion wise. The mixture was stirred at RT for 90 min, acidified with 3 M HCl (4 mL) and stirred further 15 min. The reaction mixture was added to saturated NaHCO₃ solution (200 mL) and extracted 5× with CH₂Cl₂. The combined organic phases were dried over Na₂SO₄ and the solvents were evaporated in vacuo. The resulting N-Methyl-N-boc-N′-tmob-ethylenediamine (5a) was completely dried in high vacuum and used in the next reaction step without further purification.

Yield: 3.76 g (11.48 mmol, 89% purity, 5a: double Tmob protected product=8:1)

MS: m/z 355.22=[M+H]⁺, (calculated=354.21).

To a solution of 5a (2 g, 5.65 mmol) in CH₂C₂ (24 ml) COMU (4.84 g, 11.3 mmol), N-Fmoc-N-Me-Asp(OBn)-OH (2.08 g, 4.52 mmol) and collidine (2.65 mL, 20.34 mmol) were added.

The reaction mixture was stirred for 3 h at RT, diluted with CH₂Cl₂ (250 mL) and washed 3× with 0.1 M H₂SO₄ (100 ml) and 3× with brine (100 ml). The aqueous phases were re extracted with CH₂Cl₂ (100 ml). The combined organic phases were dried over Na₂SO₄, filtrated and the residue concentrated to a volume of 24 mL. 5g was purified using flash chromatography.

Yield: 5.31 g (148%, 6.66 mmol)

MS: m/z 796.38=[M+H]⁺, (calculated=795.37).

To a solution of 5g (5.31 g, max. 4.51 mmol ref. to N-Fmoc-N-Me-Asp(OBn)-OH) in THF (60 mL) DBU (1.8 mL, 3% v/v) was added. The solution was stirred for 12 min at RT, diluted with CH₂Cl₂ (400 ml) and washed 3× with 0.1 M H₂SO₄ (150 ml) and 3× with brine (150 ml). The aqueous phases were re extracted with CH₂Cl₂ (100 ml). The combined organic phases were dried over Na₂SO₄ and filtrated. 5h was isolated upon evaporation of the solvent and used in the next reaction without further purification.

MS: m/z 574.31=[M+H]⁺, (calculated=573.30).

5h (5.31 g, 4.51 mmol, crude) was dissolved in acetonitrile (26 mL) and COMU (3.87 g, 9.04 mmol), 6-tritylmercaptohexanoic acid (2.12 g, 5.42 mmol) and collidine (2.35 mL, 18.08 mmol) were added. The reaction mixture was stirred for 4 h at RT, diluted with CH₂Cl₂ (400 ml) and washed 3× with 0.1 M H₂SO₄ (100 ml) and 3× with brine (100 ml). The aqueous phases were re extracted with CH₂Cl₂ (100 ml). The combined organic phases were dried over Na₂SO₄, filtrated and 5i was isolated upon evaporation of the solvent. Product 5i was purified using flash chromatography.

Yield: 2.63 g (62%, 94% purity)

MS: m/z 856.41=[M+H]⁺, (calculated=855.41).

To a solution of 5i (2.63 g, 2.78 mmol) in i-PrOH (33 mL) and H₂O (11 mL) was added LiOH (267 mg, 11.12 mmol) and the reaction mixture was stirred for 70 min at RT. The mixture was diluted with CH₂Cl₂ (200 ml) and washed 3× with 0.1 M H₂SO₄ (50 ml) and 3× with brine (50 ml). The aqueous phases were re-extracted with CH₂Cl₂ (100 ml). The combined organic phases were dried over Na₂SO₄, filtrated and 5e was isolated upon evaporation of the solvent. 5e was purified using flash chromatography.

Yield: 2.1 g (88%)

MS: m/z 878.4=[M+Na]⁺, (calculated=878.40).

To a solution of 5e (170 mg, 0.198 mmol) in anhydrous DCM (4 mL) were added DCC (123 mg, 0.59 mmol), and a catalytic amount of DMAP. After 5 min N-hydroxy-succinimide (114 mg, 0.99 mmol) was added and the reaction mixture was stirred at RT for 1 h. The reaction mixture was filtered, the solvent was removed in vacuo and the residue was taken up in 90% acetonitrile plus 0.1% TFA (3.4 ml). The crude mixture was purified by RP-HPLC. Product fractions were neutralized with 0.5 M pH 7.4 phosphate buffer and concentrated. The remaining aqueous phase was extracted with DCM and 5f was isolated upon evaporation of the solvent.

Yield: 154 mg (81%)

MS: m/z 953.4=[M+H]⁺, (calculated=953.43).

### Example 6

### Synthesis of N^{εA9/A17/B9}-Relaxin Mono-Linker Conjugate 6

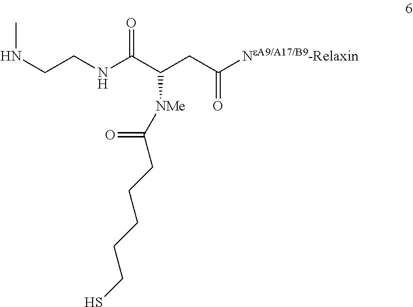

N^{εA9/A17/B9}-Relaxin mono-linker conjugate 6 was prepared by diluting 1.79 mL of a 50 mg/mL solution of relaxin H2 TFA salt (13.0 μmol, 1 eq) with 1.79 mL DMSO and 3.22 mL of borate buffer/DMSO mixture (1:1.25 (v/v) 0.375 M boric acid, adjusted to pH 8.5 with tetrabutylammonium hydroxide 30-hydrate/DMSO). The mixture was stirred for 15 min at RT and 545 μL of an 18 mg/mL solution of 5f in DMSO was added (10.4 μmol, 0.8 eq). It was stirred for further 15 min after which 8.93 mL ice cold 10% (v/v) acetic acid was added. The mixture of protected mono-linker conjugates together with unreacted relaxin H2 was isolated from the reaction mixture by RP HPLC. Re-isolated relaxin H2 (26.7 mg, 3.89 μmol) was reacted in a second conjugation reaction with 5f (3.12 μmol, 0.8 eq) according to the procedure described above.

Yield (combined): 46.9 mg (46%)

MS: m/z 1701.08=[M+4H]⁺, (calculated=1701.27).

Removal of protecting groups was affected by dissolving 44.7 mg (5.71 μmol, 1.0 eq) lyophilized product fractions in 0.89 mL of HFIP/TES/H₂O 39/1/1 (v/v/v) and stirring for 5 min at RT. 59 μL TFA was added after which the mixture was stirred for 85 min at RT. The solvent was evaporated and the mixture of deprotected mono-linker conjugates 6 was isolated from the reaction mixture by RP HPLC.

Yield: 35.7 mg (86%)

MS: m/z 1570.51=[M+4H]⁺, (calculated=1570.63).

### Example 7

### Preparation of Relaxin-Linker-Hydrogel 7

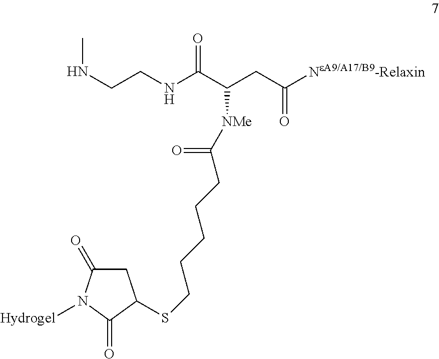

A suspension of maleimide functionalized hydrogel 4 (1.78 g, 10.3 μmol maleimido groups) in sodium succinate buffer (pH 3.0, 20 mM; 1 mM EDTA, 0.01% Tween-20) was filled into a syringe equipped with a filter frit. A solution of relaxin-linker-thiol 6 (26.8 mg, 3.7 μmol) in 1.0 mL sodium succinate buffer (pH 3.0, 20 mM; 1 mM EDTA, 0.01% Tween-20) was added and the suspension was stirred for 1 min at RT. The pH of the suspension was adjusted to pH 3.8 by addition of sodium succinate buffer (pH 4.4, 250 mM; 1 mM EDTA, 0.01% Tween-20) after which the sample was incubated at RT for 2.5 h. Consumption of thiol was monitored by Ellman test. The hydrogel was washed 10 times with sodium succinate buffer (pH 3.0, 20 mM; 1 mM EDTA, 0.01% Tween-20) and 3 times with sodium succinate buffer (pH 3.0, 20 mM; 1 mM EDTA, 0.01% Tween-20) containing 10 mM 2-mercaptoethanol. Finally, the hydrogel was suspended in the 2-mercaptoethanol containing buffer and incubated for 3 h at RT. The buffer was exchanged after 15, 30 and 60 min.

Relaxin-linker-hydrogel 7 was washed 10 times with succinate buffer (pH 3.0, 20 mM; 1 mM EDTA, 0.01% Tween-20) and 5 times with sodium acetate buffer (pH 4.5, 25.7 mM acetate, 15.4 g/L glycerol, 3.0 g/L L-methionine, 2.7 g/L m-cresol, 3.0 g/L poloxamer 188). Relaxin content was determined by quantitative amino acid analysis after total hydrolysis under acidic conditions.

Yield: 1.80 g

Relaxin loading of 7: 10.5 mg relaxin/g relaxin-linker-hydrogel

### Example 8

### Release Kinetics In Vitro (pH 7.4/37° C.)

Relaxin-linker-hydrogel 7 (containing 0.4 mg relaxin-2) was filled into syringes equipped with filter frits, washed 3 times with sodium phosphate buffer (pH 7.4, 60 mM sodium phosphate, 3 mM EDTA, 0.01% Tween-20), and incubated at 37° C. At time points the supernatant was expelled, weighed and fresh sodium phosphate buffer (pH 7.4, 60 mM sodium phosphate, 3 mM EDTA, 0.01% Tween-20) was added to the hydrogel again. Quantification of relaxin content in the supernatant was achieved by RP-HPLC/ESI MS and comparison with a relaxin standard curve. FIG. 2 shows a plot of relaxin release at pH 7.4 and 37° C. against incubation time.

Curve-fitting software was applied to estimate the corresponding halftime of release. A halftime of 6.7 d for the relaxin release was determined.

### Example 9

### Pharmacokinetics Study in Rat (HDP P14.0011)

The pharmacokinetics of 7 were determined by measuring plasma relaxin concentrations over a period of 14 days in healthy rats.

5 Wistar rats (appr. 250 g body weight) received a single subcutaneous injection of 200 μL of test item 7 in sodium acetate buffer pH 4.5, containing 2.1 mg relaxin (approx. 8.4 mg/kg). Per animal and time point 250 μL of blood was withdrawn from the sublingual vein to obtain about 100 μL Li-Heparin plasma. Samples were collected 3 days before and 2 h, 8 h, 1 d, 2 d, 4 d, 7 d, 9 d, 11 d and 14 d after test item administration. Plasma samples were frozen and stored at −80° C. until analysis. The relaxin content of the plasma samples was measured using a human relaxin-2 Quantikine® ELISA kit (R&D Systems, Minneapolis, USA) following the manufacturer's instructions. The kit standard's calibration curve was fitted using a four parameter logarithmic fit (log(agonist) vs. response with 1/Y² weighing—Graph Pad Prism software 5.02). Before analysis plasma samples were vortexed, centrifuged for 4 min in a tabletop centrifuge at 5° C. and diluted in reaction tubes (from 1:100 to 1:1000 with Diluent RD6-6). For analysis OD at 450 nm was measured with a microtiter plate reader (Tecan infinite m200) with reference wavelength correction at 540 nm. Four out of five animals showed evaluable pharmacokinetic profiles. The mean relaxin plasma levels over 14 days of these four animals are shown in FIG. 3. After a single subcutaneous injection of 200 μL 7 that contained 2.1 mg relaxin plasma levels rose to a maximum of 29.9±8.3 ng/mL relaxin at day 2. The plasma concentration subsequently decreased continuously within two weeks. The terminal half-life was determined to be 8.2 d (95% confidence interval: 6.0—13.0 d; mathematical fit: one phase decay from day 2, constrain plateau=0; —Graph Pad Prism software 5.02).

### Example 10

### Preparation of Relaxin-Linker-4-Arm-PEG 8

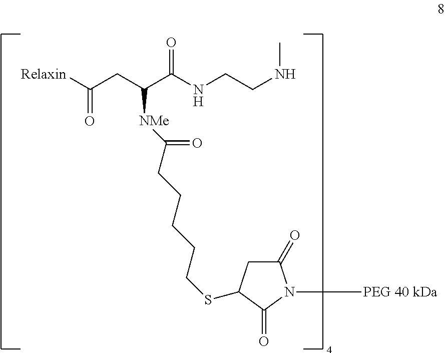

Relaxin-linker-4-arm-PEG 8 is prepared by dissolving 68 mg 40 kDa 4-arm PEG maleimide (1.7 μmol, 1.0 eq) in 0.5 mL water. Relaxin-linker-thiol 6 (60 mg, 7.7 μmol, 4.5 eq) is dissolved in 2.0 mL sodium succinate buffer (pH 3.0, 20 mM). The relaxin-linker-thiol solution is added to the 4-arm PEG-maleimide solution. The pH is adjusted to pH 4.0 by addition of sodium succinate buffer (pH 4.4, 0.25 M). The mixture is stirred for 3 h at RT after which the pH is adjusted to pH 3.0 by addition of 0.2 M HCl. The mixture is purified by ion-exchange chromatography and desalted by gel filtration chromatography.

Relaxin content is determined by quantitative amino acid analysis after total hydrolysis under acidic conditions.

### ABBREVIATIONS

AcOH acetic acid
Bn benzyl
Boc t-butyloxycarbonyl
COMU (1-Cyano-2-ethoxy-2-oxoethylidenaminooxy)dimethylamino-morpholino-carbenium hexafluorophosphate
DBU 1,3-diazabicyclo[5.4.0]undecene
DCC N,N,-dicyclohexylcarbodiimid
DCM dichloromethane
DIEA diisopropylethylamine
DMAP dimethylamino-pyridine
DMF N,N-dimethylformamide
DMSO dimethylsulfoxide
EDC 1-Ethyl-3-(3-dimethylaminopropyl)carbodiimid
EDTA ethylenediaminetetraacetic acid
eq stoichiometric equivalent
ESI-MS electrospray ionization mass spectrometry
EtOH ethanol
Fmoc 9-fluorenylmethoxycarbonyl
HATU O-(7-Azabenzotriazol-1-yl)-N,N,N′,N′-tetramethyluronium hexafluorophosphate
HFIP hexafluoroisopropanol
HPLC high performance liquid chromatography
HOBt N-hydroxybenzotriazole
iPrOH 2-propanol
Mal 3-maleimido propyl
Mal-PEG6-NHS N-(3-maleimidopropyl)-21-amino-4,7,10,13,16,19-hexaoxa-heneicosanoic acid NHS ester
Me methyl
MeOH methanol
MS mass spectrum/mass spectrometry
MTBE methyl tert.-butyl ether
MW molecular mass
NHS N-hydroxy succinimide
NMP N-Methyl-2-pyrrolidone
OtBu tert.-butyloxy
PEG poly(ethylene glycol)
RP-HPLC reversed-phase high performance liquid chromatography
rpm rounds per minute
RT room temperature
SEC size exclusion chromatography
TES triethylsilane
TFA trifluoroacetic acid
THF tetrahydrofurane
TMEDA N,N,N′N′-tetramethylethylene diamine
Tmob 2,4,6-trimethoxybenzyl
Trt triphenylmethyl, trityl
UPLC ultra performance liquid chromatography
UV ultraviolet

1. A carrier-linked relaxin prodrug or pharmaceutically acceptable salt thereof comprising at least one relaxin moiety covalently connected to a carrier moiety via a reversible linker moiety.

2. The prodrug of claim 1 or 2, wherein the carrier-linked relaxin prodrug comprises, preferably is, a moiety D-L, wherein
(i) -D is a relaxin moiety;
and
(ii) -L comprises, preferably is, a reversible linker moiety -L¹ represented by formula (I), 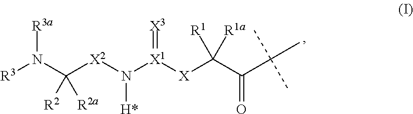
wherein the dashed line indicates the attachment to a nitrogen of D by forming an amide bond;
X is C(R⁴R^{4a}); N(R⁴); O; C(R⁴R^{4a})—C(R⁵R^{5a}); C(R⁵R^{5a})—C(R⁴R^{4a}); C(R⁴R^{4a})—N(R⁶); N(R⁶)—C(R⁴R^{4a}); C(R⁴R^{4a})—O; O—C(R⁴R^{4a}); or C(R⁷R^{7a});
X¹ is C; or S(O);
X² is C(R⁸R^{8a}); or C(R⁸R^{8a})—C(R⁹R^{9a});
X³ is O; S; or N—CN;
R¹, R^{1a}, R², R^{2a}, R⁴, R^{4a}, R⁵, R^{5a}, R⁶, R⁸, R^{8a}, R⁹, R^{9a} are independently selected from the group consisting of H; and C₁₋₆ alkyl;
R³, R^{3a} are independently selected from the group consisting of H; and C₁₋₆ alkyl, provided that in case one of R³, R^{3a} or both are other than H they are connected to N to which they are attached through an SP³-hybridized carbon atom;
R⁷ is N(R¹⁰R^{10a}); or NR¹⁰—(C═O)—R¹¹;
R^{7a}, R¹⁰, R^{10a}, R¹¹ are independently of each other H; or C₁₋₆ alkyl;
Optionally, one or more of the pairs R^{1a}/R^{4a}, R^{1a}/R^{5a}, R^{1a}/R^{7a}, R^{4a}/R^{5a}, R^{8a}/R^{9a} form a chemical bond;
Optionally, one or more of the pairs R¹/R^{1a}, R²/R^{2a}, R⁴/R^{4a}, R⁵/R^{5a}, R⁸/R^{8a}, R⁹/R^{9a} are joined together with the atom to which they are attached to form a C₃₋₇ cycloalkyl; or 4- to 7-membered heterocyclyl;
Optionally, one or more of the pairs R¹/R⁴, R¹/R⁵, R¹/R⁶, R¹/R^{7a}, R⁴/R⁵, R⁴/R⁶, R⁸/R⁹, R²/R³ are joined together with the atoms to which they are attached to form a ring A;
Optionally, R³/R^{3a} are joined together with the nitrogen atom to which they are attached to form a 4 to 7 membered heterocycle;
A is selected from the group consisting of phenyl; naphthyl; indenyl; indanyl; tetralinyl; C₃₋₁₀ cycloalkyl; 4- to 7-membered heterocyclyl; and 9- to 11-membered heterobicyclyl; and
wherein L¹ is substituted with one to four moieties L²-Z and wherein L¹ is optionally further substituted, provided that the hydrogen marked with the asterisk in formula (I) is not replaced by L²-Z or an optional further substituent;
wherein
L² is a single chemical bond or a spacer; and
Z is a carrier.

3. The prodrug of claim 1 or 2, wherein the relaxin moiety is human relaxin-2 moiety comprising an A-chain of SEQ ID NO:1 and a B-chain of SEQ ID NO:2.

4. The prodrug of claim 2 or 3, wherein L²-Z is attached to R¹, R^{1a}, R², R^{2a}, R³, R^{3a}, R⁴, R^{4a}, R⁵, R^{5a}, R⁶, R^{7a}, R⁸, R^{8a}, R⁹ or R^{9a} of formula (I).

5. The prodrug of any one of claims 2 to 4, wherein X is C(R⁷R^{7a}).

6. The prodrug of any one of claims 2 to 5, wherein X¹ is C.

7. The prodrug of any one of claims 2 to 6, wherein X² is C(R⁸R^{8a}).

8. The prodrug of any one of claims 2 to 7, wherein L¹ is of formula (IV): 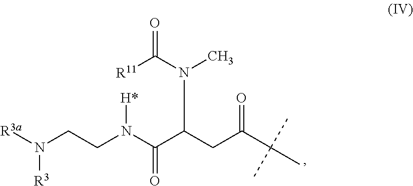
wherein
the dashed line indicates the attachment to a nitrogen of D by forming an amide bond;
R³ and R^{3a} are used as defined in formula (I);
R¹¹ is C₁₋₆ alkyl;
and wherein L¹ is optionally further substituted, provided that the hydrogen marked with the asterisk in formula (IV) is not replaced by a substituent.

9. The prodrug of any one of claims 2 to 8, wherein L² is of formula (Ia): 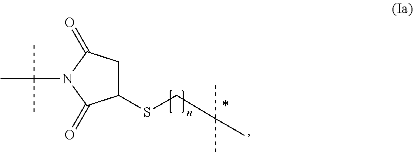
wherein
the dashed line marked with the asterisk indicates attachment to L¹ and the unmarked dashed line indicates attachment to Z; and
n is 1, 2, 3, 4, 5, 6, 7, 8, 9, 10, 11, 12, 13, 14 or 15.

10. The prodrug of any one of claims 1 to 9, wherein the carrier is water-soluble.

11. The prodrug of any one of claims 1 to 9, wherein the carrier is water-insoluble.

12. The prodrug of claim 11, wherein the carrier is a hydrogel.

13. The prodrug of claim 11 or 12, wherein the prodrug is in the form of a microparticle.

14. The prodrug of any one of claims 1 to 13, wherein the half-life of the carrier-linked relaxin prodrug of the present invention after subcutaneous injection is at least 20 times longer than the half-life of intravenously administered native relaxin-2.

15. A pharmaceutical composition comprising at least one prodrug of any one of claims 1 to 14.

16. The prodrug of any one of claims 1 to 14 or the pharmaceutical composition of claim 15 for use in a method of treatment of a disease which can be treated with relaxin.

17. The prodrug of claim 16, wherein the disease is heart failure.

18. The prodrug of claim 16, wherein the disease is pulmonary hypertension.
